(19) United States
(12) Patent Application Publication
(10) Pub. No.: US 2021/0285063 A1
(43) Pub. Date: Sep. 16, 2021

(54) GENOME-WIDE MAIZE SNP ARRAY AND USE THEREOF

(71) Applicant: Beijing Academy of Agriculture and Forestry Sciences, Beijing (CN)

(72) Inventors: Fengge Wang, Beijing (CN); Hongli Tian, Beijing (CN); Jiuran Zhao, Beijing (CN); Yang Yang, Beijing (CN); Hongmei Yi, Beijing (CN); Liwen Xu, Beijing (CN); Rui Wang, Beijing (CN); Lu Wang, Beijing (CN); Jianrong Ge, Beijing (CN); Yaming Fan, Beijing (CN)

(73) Assignee: Beijing Academy of Agriculture and Forestry Sciences, Beijing (CN)

(21) Appl. No.: 17/105,847

(22) Filed: Nov. 27, 2020

(30) Foreign Application Priority Data
Nov. 28, 2019 (CN) 201911186629.9

(51) Int. Cl. C12Q 1/6895

(52) U.S. Cl. CPC C12Q 1/6895; C12Q 2600/156

(57) ABSTRACT

The present disclosure provides a genome-wide maize SNP array and use thereof. In the present disclosure, by means of large-scale sequencing mining, aiming at the problems of the existing high-density arrays of maize, 400 broadly representative maize inbred lines inside and outside of China are deeply sequenced to mine excellent loci therefrom, and research results of six maize arrays are integrated; by adopting the steps of biological information data analysis, locus basic parameter evaluation, probe design evaluation, a set of SNP locus combination, containing 1,536 loci, is formed, and genotyping is realized on an Affymetrix axiom platform. The genome-wide maize SNP array of the present disclosure contains probes for the 1,536 SNP loci. The array of the present disclosure can be used in DNA fingerprint construction, cultivar identification and confirmation of maize cultivars, research on germplasm resources at a genomic level, and molecular marker-assisted selective breeding.

### TECHNICAL FIELD

The present disclosure relates to the technical field of crop breeding and molecular biology, and in particular to a genome-wide maize single nucleotide polymorphism (SNP) array and use thereof.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One-506,952 Byte ASCII (Text) file named “WANG_GWP202010421_BAAFS-P0001_17105847 PATENT-APPLICATION_REVISED-SEQUENCE-LISTING.TXT”, created on Apr. 23, 2021.

### BACKGROUND

With the rapid development of genomics and biotechnology platforms, crop genotyping has entered a new era. Various crops have successively undergone large sample resequencing to establish a genome-wide genetic information database thereof. Based on the establishment of the genome-wide genetic information database, the development of excellent molecular markers, haplotype mapping, and assessment of genome diversity have begun systematically. Molecular markers refer to DNA sequences that are heritable and detectable, are genetic markers based on nucleotide sequence variation between individuals, and are a direct reflection of genetic polymorphism at the DNA level. At the same time, high-density array technology typing platforms have been released one after another, and these platforms have been used in molecular identification, molecular breeding and basic research. SNP markers are characteristic of high distribution density and uniform distribution on the genome, and are the most suitable markers for developing arrays. Maize, as a diploid hybrid crop, has a research foundation of high recombination rate, rich genetic diversity, and excellent genomics; the development and evaluation of SNP loci thereof have an excellent research foundation.

SNP, as the third generation genetic marker, is the most abundantly distributed on the genome, and is valued by many researchers due to dimorphism, low mutation rate of a single SNP locus, and easy realization of automation and scale through array technology. High-density arrays of maize are mainly commercialized arrays, and evaluation materials thereof are basically from a few dozens of samples, which leads to the analysis of wide range materials with low data quality and low polymorphism. In order to accelerate the application of maize genetic research and breeding, it is necessary to select materials worldwide to evaluate SNPs. Use of high-quality sequencing data in combination with the research foundation of existing arrays is urgently needed to develop a genome-wide, high data sharing, and high throughput genome-wide maize SNP array suitable for multi-platform detection.

### SUMMARY

An objective of the present disclosure is to provide a genome-wide maize SNP array suitable for molecular identification and molecular assisted breeding.

In the present disclosure, 400 broadly representative inbred lines inside and outside of China are deeply sequenced, and SNP loci are mined at a genome-wide level to establish an excellent collection of SNP loci; based on biological information analysis methods, dimorphic and single-copy SNP loci are determined and high-quality loci in 6 known arrays have been integrated to form a set of candidate loci; locus scoring evaluation and probe design are performed by submitting to Affymetrix and Illumina, two mainstream technology platforms; loci that meet the design requirements are further evaluated based on the sequencing data and further analyzed to obtain a high-quality and efficient locus set based on parameters such as polymorphism, heterozygosity, and conservation of flanking 60 bp sequences; the above-mentioned loci are formed into a set of candidate loci according to the principle of substantially uniform distribution; locus scoring evaluation and probe design are performed again by submitting to Affymetrix and Illumina again to form a high-density 60K array (Maize6H-60K); based on the 60K array, using 200 inbred lines and 200 hybrids, loci are verified according to the test results such as data acquisition rate, genotyping effect, and characteristics of triplet, and marker loci with 9K compatible multi-platform, high test data quality and high polymorphism are selected from the 60K arrays; still further, in accordance with a uniform distribution, 2-4 markers are screened each 2,000 Kbp, and finally, 1,536 SNP markers are determined from 8K. The specific information of 1,536 SNP markers is shown in Table 1.

TABLE 1

| Lo- | Chro- | | |
|---|---|---|---|
| cus | mo- | Ph]sical | |
| No. | some | location | Flanking sequence |
| MI00 | 1 | 379843 | CAGCCATAAAACGAGATGCTCGACATCCTGATGCT[C/T]GAATGGTGATTATTAAAAAA |
| 01 | | | CAAGGAAATCAAAGA |
| MI00 | 1 | 1747250 | ACATTCCGATCGACGCTGAGAAGTTGCAACCCCAG[A/C]AAGGGTCCTCGACCCATGT |
| 02 | | | CTCGCAGGTACAACCA |
| MI00 | 1 | 2873870 | CGAACGGTTGGACACACTGTGCTGAAATGGCGTGA[C/T]GGATACAGTGACACATTAC |
| 03 | | | TTGCACATACATACTG |
| MI00 | 1 | 7042592 | CCGATACAAAGACGAATAGGTACAGTAATCACCTC[C/T]TCTTTTCCTCTCTTGAGACG |
| 04 | | | TGGCATATATTTTTT |
| MI00 | 1 | 7186768 | ACATTTCCAGCAGTGGCGGCCAAGGCTCGGATGTT[C/T]TCCTGTGTGTCAAAAAAAC |
| 05 | | | CCATTTCTTGGAGCTG |
| MI00 | 1 | 7566162 | CGAAATTGTTTGTGAATCTAGTCTGTTTACTTCCA[G/T]GCTTACGTGCGGATGTGGCC |
| 06 | | | AGGAGCGTTGGCCAG |
| MI00 | 1 | 7819799 | CAAAACGGGGTGGGGTTTTCATTTTATTACACGAC[A/G]AACAGAATCAGCAAACGAC |
| 07 | | | GGAATGGACTCGTAGG |
| MI00 | 1 | 8974336 | CTTGCCTGGTACAGGAACTGTGCGTAGTTCCTGAG[A/G]AAGAGGCCGTTGCAGGGG |
| 08 | | | TCCTCCTCGATCATCTT |
| MI00 | 1 | 10435348 | GGAGACTTCGATCATGGCTTAAGATGAACCATGGC[A/G]ACATGCTTAACACATGCAA |
| 09 | | | GTCAAACGAAAAGTAG |
| MI00 | 1 | 12241206 | TGAGACCCTGGGAGTAGTCTAGGAGAGAGAAGGTC[A/G]TCATCTGCATGCATGGCA |
| 10 | | | GTATGGCACGCAGTGTG |
| MI00 | 1 | 13832598 | TATATTTAGTCAGTCAGCAGATTAGGAATAAAAGA[A/G]ACAACAGATAAGTTTTTATA |
| 11 | | | TTTAGTCAGTCTGTT |
| MI00 | 1 | 15067243 | CGACTAAAAAACAACACATGGCAGAGGCAGACCAC[A/C]ATTTGAACTTCCCTCCAA |
| 12 | | | AAGTCCAGACTCCCAAG |
| MI00 | 1 | 15271845 | TTGCGTTGTTGCTCATGTATCGCCTCAAGAGGGGA[A/C]GCTTAAAGATGGGTGGAAG |
| 13 | | | CCGATACACCGTATTC |
| MI00 | 1 | 16059383 | CTCAGCCCCTCCACTCGCATCCCGTGTCCATCGGC[A/C]GGAGATCAAACTCCACACG |
| 14 | | | GCGCTCTGCTTCCATT |
| MI00 | 1 | 16125470 | TGGAGAGAATCCATGTACCTGAAAAGCTGCCGAAA[A/G]TATAGCAAGAGCAGGGCA |
| 15 | | | AAATGTTGAAAATTTTT |
| MI00 | 1 | 16585784 | AGCCATGCATGATTGGTACTCGCTCCATTGAGCTC[A/G]ACAAAATCATCGAAGATTAG |
| 16 | | | CATCTCCTTGAACCA |
| MI00 | 1 | 19592715 | AGTTTAATTAAGGAATCAAAAGCATAAATCACACA[C/T]ATAAAGAATTGAAATTTAAA |
| 17 | | | ATGGACGCTTATTAA |
| MI00 | 1 | 19943737 | AGACTGCTTTCTTTCAATGGCTCCTGACTGCCTTC[G/T]TTCGATGCCTCCTGACTGCC |
| 18 | | | TTCTTTCGATGCTGG |
| MI00 | 1 | 20644157 | TCACCTGCTATGTTTTTCTAGGTTGTGCCATTAGA[C/T]CAGGCACACAAAATATACAA |
| 19 | | | CGCCTTGAAAGAGAA |
| MI00 | 1 | 22614426 | AGTTAAACGGTTTATGAAAATAAGTTGACGGCCAA[A/G]ACATGCAGCCAATTTAAAA |
| 20 | | | ATAAATGTAGGCACCA |
| MI00 | 1 | 23193957 | GCAGAAAGAAAAATCGGAAAGAAAGCCGGCCCCGG[C/G]CGGATGCGGTTTCTCGA |
| 21 | | | GAATCTTCTGGCAACGAC |
| MI00 | 1 | 24044764 | ACCTGTAGTGTTAGCATGAGTCATGATACCATGTT[A/G]GACATTTGAAGGAAAGTTCT |
| 22 | | | AATAGATAAGTTCGC |
| MI00 | 1 | 24053957 | GACCAGTGATCAACTGCATCGCGACGGCCCATGCG[C/T]GTCATCAAGATGCGGCCCA |
| 23 | | | CACGCACGCGTACCTA |
| MI00 | 1 | 24209878 | TCTTCCCAAGGGGATGGGAATGAGGAGTCATTCAC[C/T]GAATAAGAATTCTCCGCTG |
| 24 | | | CCATCTCCAGTGGGGA |
| MI00 | 1 | 24411696 | GGGAGCAGATCCCACCTTTCATTAAAATACCATCA[C/G]GGCATCTGAGTCAGATGCCT |
| 25 | | | GACGATGGACAAAAG |
| MI00 | 1 | 24938795 | TAACAGAAACCGAACAAAAAAGGGGGAATCCTAAC[G/T]AACTGCGCCCTGATCGGG |
| 26 | | | GGCGCAATCCTATATGG |
| MI00 | 1 | 25641942 | TAATAATACAATAAGTGTGTGCACACGAATAAATG[C/T]CATCATTGTCAAGTGTTAATT |
| 27 | | | TGAATGACAAAGAT |
| MI00 | 1 | 26916675 | AATTAATGGCGCAAGGTTCCTGCTTGTGCAATTTG[A/C]TCATTTCATGAGGGCATGAG |
| 28 | | | TAGTCATCACCATGC |
| MI00 | 1 | 27628871 | CGTGGTCACTGGCCACTGCACATTGCCCAGGATTT[C/T]CTGTGGATGGCCGGGCCCG |
| 29 | | | GGACAGCAGCATGGTG |
| MI00 | 1 | 28388021 | GTTCGTCTCTACGTGACTCTCTCTCCATATATATA[G/T]TCTCTTAATTACACTTATGGT |
| 30 | | | TTAGAGGCTCTAGG |
| MI00 | 1 | 28864842 | AGGGAGTAATCCAATGCTACTGGTGTTTGTGCACC[A/G]ACATGTGAACCTTTTTGTGT |
| 31 | | | CTAACTGATGCTGCC |
| MI00 | 1 | 30282315 | CCTTTCATCACGCTGGCGGGTCCGGGATTGCTGTT[C/T]GGAGTACGCGCTCCTTTCA |
| 32 | | | AAATGTTTGGTGAATT |
| MI00 | 1 | 30542690 | CCTTCTGAGCTCTGTGGTGCTCTTATAGGTTTTTT[C/G]AGAGGTAGCTGTGGGGTCCT |
| 33 | | | TTAATTTAGGTCCTA |
| MI00 | 1 | 31214698 | TCACGCGGTGTCCGATTGATACCATCGTCGTTGCT[A/G]CAGGTACGGCTCGTGATAG |
| 34 | | | ACCCGGTTATAACGAA |
| MI00 | 1 | 31654357 | GTATATACACATCGGCACTTCCTAATCTTCGCTTC[A/G]GAATGCCAATTTGGCTCCTTT |
| 35 | | | TTCCCTTAATCATA |
| MI00 | 1 | 33003573 | AGCAGAGGATTATAGCTTTCATTGGAAGGTATGCA[A/G]TGGTGGCATAACTTTTATGG |
| 36 | | | CGATCATTAATTCTG |
| MI00 | 1 | 33835829 | TCGCCATGGCCGGAAATTGTTTCCGGGGGGTTTCT[A/G]CCTTTCTGGCTTGGATAGA |
| 37 | | | GGAAAGAGATAGAGCG |
| MI00 | 1 | 35804891 | AGGACTAAATGTGATGCTATCACACCACAAAAGAA[C/T]GTGGCACCGAGCATTACGA |
| 38 | | | TCCTGAGGTCGGGACT |
| MI00 | 1 | 35995804 | AAATCATTCTAGGTGAGGTATGGCTTCAAATTCCC[C/T]GTCCTCCTCTTATTTAAAAA |
| 39 | | | AAAGGACATGTAATT |
| MI00 | 1 | 37159651 | TGCCTGAAGACACATATCAGGTGGCGTGTTCTCTA[A/G]GGCAGAAGACTACCGACCT |
| 40 | | | AGCCGAACTAAAGGAA |
| MI00 | 1 | 38255163 | CATACTGGACCTTCGATTTACCAATCTGTCCATTA[C/T]GATCAGCGATTAAACCGTAA |
| 41 | | | GGTTTAGGAAATTTC |
| MI00 | 1 | 39750051 | TTTTGTTCCTGTACATGTGAAGGTCAATTGGTCTG[A/G]GGTTTTTAAAATACAGTAAT |
| 42 | | | TGGTTTACGGGTTTT |
| MI00 | 1 | 40231408 | TTTTAACTGCATTGCGGAGTCAGTCAGGTGGGCTG[G/T]CATCTGCGCAAAAGGTGAA |
| 43 | | | ACCATAGGCCCGGTGC |
| MI00 | 1 | 40905538 | TATTGTGGTAATTAAGAAAAAAACAGGGCTGCGTC[C/T]AAGCAGTTGTACATAGAAG |
| 44 | | | ATTAATATCCGATGAT |
| MI00 | 1 | 41088075 | CCGGGTGTGACATCTAGGGCCTTTGACACCACACA[C/T]TCAATAAGGTTATTCGTAA |
| 45 | | | AAGTTAATTACTTATG |
| MI00 | 1 | 41473420 | GGCCCTACCTGCCAGCCACACCCGATCGTCCTCCT[G/T]GCGTACCTAACGCATGCCT |
| 46 | | | AATCGCGGGAGTAAAC |
| MI00 | 1 | 43979495 | CTGGCAATGTGTTCTCATACGTAATCTGGAAGTAC[A/G]GTGTCTTGGTATTTCCGTCT |
| 47 | | | GCTTCCGAGCCAACG |
| MI00 | 1 | 47295429 | TTGGGTGTACATGTGTACACCCATGCCCCTTTACT[G/T]GTTCCGCCCCCGTAAAGACT |
| 48 | | | AAGCTGAAACCATGG |
| MI00 | 1 | 47508846 | CCGCCTCTCCGCGTCCGCTGAGTCACTGCCTCCTG[A/G]GCCCGGACCCACACGCCCC |
| 49 | | | TCGTCAATCAACATCA |
| MI00 | 1 | 47516308 | TTACAGAACTTATGCTCTCCTTCTCAGAGCTTGTG[C/T]AAAATGCTCAGACCTCTATG |
| 50 | | | CTTCCATTGAATCCA |
| MI00 | 1 | 48685205 | CATGTGACCATAGTTCTAAGTTGTACCTACGCCCA[A/G]GTGAATATGGCACAGGATTC |
| 51 | | | AAGATCTGCCCTTGT |
| MI00 | 1 | 48874576 | AACATGATGATGGGGAAATTATCTGTGACATACCC[C/T]TATAAACTTTTAACATGATGG |
| 52 | | | GCAAATTATCTGTG |
| MI00 | 1 | 49374234 | TTTCTCTTGGCATCCATCCTTCTATTTTTCTCCTC[A/G]ATTCTAATTCCACTGTCCTTT |
| 53 | | | TTGAGCCTCTCGCT |
| MI00 | 1 | 50777654 | CCCGAGAGGGACTCCATTGGGAGCAAGGATCTTGG[A/G]GTTGCTTCGGGATCAGCA |
| 54 | | | GGCCAACCAAAATACTT |
| MI00 | 1 | 51296270 | ATATATATAAATGACTAATCGATCTTCCTTAAAAG[A/G]CACACGCATGGTTTTAAATGA |
| 55 | | | TGACAGGCTAGCTT |
| MI00 | 1 | 51789297 | AGGGATAACGAAAACCTTGAGATGACGGCGGGGCA[A/G]TTGTACCACTTCCCTGGA |
| 56 | | | CGACTTCCCCTAGAGGG |
| MI00 | 1 | 52862609 | CTCCTTTTCAATACACCTTTCTTTTTTCAGTACAT[C/T]TATATCTCTACTCTTTGAGAA |
| 57 | | | GTATTTAGGTCGTT |
| MI00 | 1 | 55757457 | GGATTTCATGTGAGTACTTGCAACAGTTATGGCAT[C/T]GTGAGAATCCATGATATGCC |
| 58 | | | ACTGAATAACTACAC |
| MI00 | 1 | 56074772 | GAAGAAGGAAGACTCGCAAACCCGACGCAGCGCAT[A/G]GGCGCGCAGGCGCACCT |
| 59 | | | GCCTCAAAAGCACCGACG |
| MI00 | 1 | 58651548 | TCTCGCTATCAAGATCTAGCATTTGCTGAAAGATG[C/T]ATGGATGTGCAGATTGGTAA |
| 60 | | | ACATTGTATAATGGA |
| MI00 | 1 | 59510464 | CGCAAGGGTGAAATTGAGTTCTACAAGTTACCCCG[A/G]GGTAGAGGTAGAGGAAGA |
| 61 | | | GGAAGTGGAAAATCGAA |
| MI00 | 1 | 61452535 | CCACGGTAGTGTCAATTTGAGAACAAAAATTTCAA[A/C]GGGTGTTCAAAGAATGAA |
| 62 | | | GATTTGAAGTTCAGATG |
| MI00 | 1 | 61478195 | GTCGCTCCTGGACGGTGTAGCCCTTCTGACGTCGT[C/T]CGTAGGGTCATGTGTAGCC |
| 63 | | | ATACTCCTGGCATGGC |
| MI00 | 1 | 62702677 | GCGTAGGGACGACACTACATGATTGTGGATCTACT[G/T]AATGAAGAAAAAAAATTAC |
| 64 | | | TGCATCGGTGCTGTAA |
| MI00 | 1 | 63036079 | TAACACGGTTTGATAGGATTTTAAGATTTTTGTTG[G/T]AAAACCGCTCGTGCTGAAAA |
| 65 | | | ATGGCTGAATTCTTA |
| MI00 | 1 | 63360371 | TTGAGTTCTTTGGCAGGCCACATTAACCTAGTGCA[A/G]TTTTATGATGCTTTTGAGGA |
| 66 | | | TGAAGAAAATGTATA |
| MI00 | 1 | 68935556 | ACTGCTTCTCAAGTCACTATAAGATATACATGAAG[A/G]AATAAAACAAGACAAAAAA |
| 67 | | | CAAGCTTCATCACACC |
| MI00 | 1 | 69652284 | AGAAGGCGCCAAGGCCACTGCTAGAGCCGTCATGG[C/T]AGGACAACATTTTTGCGT |
| 68 | | | TGTATAACCCGCAAGGT |
| MI00 | 1 | 72711033 | ACCGACTCTTCACCAGTTTCCTTGTTTCAGAGAAA[A/G]ACAGTTCCTTCCTCAACCA |
| 69 | | | CTTTCAGTCTTACCAG |
| MI00 | 1 | 75397953 | GGGTAGAAAATCGAGTGTAATTAAGATAACGACTT[C/G]TTGGTCCTGTAATTACCCCG |
| 70 | | | TTTGTACAGTACAAC |
| MI00 | 1 | 77043432 | CGCGTATCTTTTCTTCTTCTGTGACAAATAAAACA[A/G]CACTTTTCCGTATTTGGAAA |
| 71 | | | TCCTGACGTAAGCTG |
| MI00 | 1 | 79334351 | GTCCAACAGAAAGGCACCGGCTAATCCAATCTGAT[A/C]TGACACTAGACAGAGTTCG |
| 72 | | | AACACGGACCATACTA |
| MI00 | 1 | 83653657 | CCCTTTTTTATGTTGCTAGCTGTTGCTTGTGTTCA[A/C]AGTCCAAGATCCAAAGAACG |
| 73 | | | CACATCCTTGCCTCC |
| MI00 | 1 | 83789456 | TAGGAATCCTAATTGCACCGACGGGAAATGAAAAT[A/G]TATTTTAGTAGCGGTTCTTA |
| 74 | | | CCAAAAAATGACACT |
| MI00 | 1 | 84681472 | TTCTCCATGACAACAGCATAAACTTAAAAGGTTTC[A/G]GAACATTGTTAAATCAAAT |
| 75 | | | GAATTCCGCATCTACT |
| MI00 | 1 | 85839980 | GCTGGCCTCATACTTATGCTCGTCGCCAACGCTAC[A/C]AGCTGCAACAAGTTGATATC |
| 76 | | | AAAGAACATAACAGC |
| MI00 | 1 | 86854913 | CTACTTGTGCTTCTGTTTCTAAACTTCTCATGCTC[A/G]GTTTGCATGCAGTGCCATATA |
| 77 | | | GTGCTCCATTTCAC |
| MI00 | 1 | 88529612 | TCTAGCGTAGATAGACCCCCAACAACACCTTTCTA[A/G]AAAATGCTTGATAGGCCCAT |
| 78 | | | CCCACATGTCAAGAA |
| MI00 | 1 | 88974015 | GTTGAGTTTCTGTCGCAGCTGCAAAGATATGCTTG[C/T]GTAAACAATTCTGGTTGATC |
| 79 | | | GGCATACTACAAAGA |
| MI00 | 1 | 91276994 | TATGAAAAGTTAGTACCACTATTTAAATTATACTC[A/C]CTCCCTCCGTTCCAAAAAAA |
| 80 | | | AAAAATATTCGTTTT |
| MI00 | 1 | 91492792 | GCAACGTCAGGGTTGTGGGCTACTGGGTTGCGTTG[C/T]GCCATGCGCGTACGCTCAC |
| 81 | | | ATCCCTCGTCCAGCTA |
| MI00 | 1 | 98173865 | AGGCCGCGCAGAGAGCGACCAGCTCCCTTCCTTCC[A/C]ACTCAGCCCGATCTAATGC |
| 82 | | | CGTGTGTAGCAGTTAC |
| MI00 | 1 | 99988058 | GGATATTCTCTGGGTTAGAAATAGTCCTGGGGTCA[C/G]TACAAACCTTTAGTATGAAT |
| 83 | | | TGATCAAGTTCGTGG |
| MI00 | 1 | 10156805 | TCCTCTAGCGCCACACATGCTGCATGCTCGGATCT[C/T]CCATGCGTAGTAAAATTCTT |
| 84 | | 2 | CACCTCTCGTGTGGG |
| MI00 | 1 | 10294939 | ATGAAGCTGATGCAGAAGATGTTGCCATCTAAGCC[A/G]AAGCGATAGCTGCCAGGGT |
| 85 | | 1 | CACGAAGAGGGCTACA |
| MI00 | 1 | 10551082 | CGCACTCTACTGGTCTTCCCAGCTCCCAGATGCTC[C/T]GCCAGCATAGCCTTCACAGC |
| 86 | | 2 | CACAAAAACGTAGGT |
| MI00 | 1 | 10790560 | AACACATATTTTGTAGGTTCTATGAGCCATCATCA[A/G]AAGAACACAAACCACGTGC |
| 87 | | 1 | AGGCAATAAATAAAAT |
| MI00 | 1 | 10807025 | GCACCTTTTCTGAAATCTATCAATCCAAAGCCTCT[A/G]AGGTGCTCAAAAACATTCCT |
| 88 | | 7 | AAGGGCATTTAGAAC |
| MI00 | 1 | 11064520 | CGCTCTCAATCCCACCTGGTTGATAGCCACGGGGG[C/G]CCTGCGGATAAAATCCCGA |
| 89 | | 6 | GAAAGGATTTTCCTTG |
| MI00 | 1 | 11139061 | GACTCTGACTTTTTCCTCTGCTTCTAGTATCAAGT[C/T]AGGTCCAAAAATTTCCCTCT |
| 90 | | 3 | CTCCAGCTTGTGACC |
| MI00 | 1 | 11140153 | GCGCTCTGCTGAGGCCTTGTCATATGGTTCCTCTT[C/T]GTTCTTTATGTACCTGATGAT |
| 91 | | 9 | CGGCTCTCTCCAGT |
| MI00 | 1 | 11142507 | CCCATTGCTTGGTACCAATTCACCGCAGCTTTTAG[A/T]GGAAATTATATTCCACCGGG |
| 92 | | 2 | TCTAATGGCAATGAA |
| MI00 | 1 | 11234141 | GAGATCACTGTAGATCCTCAGCATGGACTCGACAA[C/T]CCTGTTATGTTTTGGGATGA |
| 93 | | 3 | CATTATTAAAGGTGT |
| MI00 | 1 | 11710565 | TATACTTAGATTCAAAGGGCAGGTGGGGGTGTTTC[C/T]GGATCCGAATTATGTATCGA |
| 94 | | 2 | TCTCCTTGGGCAGAA |
| MI00 | 1 | 11863509 | TAGGGGCAAGAGCATTGTCGCCACCATTGCTGGCT[A/C]AGGGCAGTCATGGTGAAC |
| 95 | | 0 | CACCGTCTCTAGACACG |
| MI00 | 1 | 14273021 | CACATGCTTTTCCTACTTCAATGTTCTTCATTTTG[C/T]TGTCACACCATTCACTACTAA |
| 96 | | 8 | CAAAGAGAACTTTC |
| MI00 | 1 | 14721174 | GCTCTAACTGTCGTGATATCGATTGTCCGACACCA[C/T]CCCCGAGGGCACGTTCGGA |
| 97 | | 9 | CCCAAGCGCGGCCTCC |
| MI00 | 1 | 15212069 | TTTGGTAGCTCCAAACATACCATGGTTTTGATAAA[C/T]AGTGTTTAGATACAACATTG |
| 98 | | 7 | TAAACTATAGTATTA |
| MI00 | 1 | 15242686 | ATTAAAAAGAGATCCCTGTTTATTTCTTGATACTC[C/T]CTGATGTGTACGTTTAATTGT |
| 99 | | 9 | GAACCAGCCCCTGC |
| MI01 | 1 | 15429503 | GCCAGAAACAGAGGGGCTCTTGTGCTAATGTGTAC[A/G]ACGAAGGGGTAAGTACAG |
| 00 | | 3 | ATCTCAGCCATCCGATT |
| MI01 | 1 | 15509730 | GTAATAAAAACTCACCACAACCCTTGGCTCTTCCG[C/T]GAGTCATTGTAATATTGTAC |
| 01 | | 0 | CGAGTGGGGCAAGAG |
| MI01 | 1 | 15792361 | TCCTCGCTCTGATCCTCCTCCAGTGCACTGCTCCG[C/T]TCCGGCGACCTCGCTGCCG |
| 02 | | 2 | GCCCGGCGCTCCCCGG |
| MI01 | 1 | 15929227 | TAGCTGGCCGAATTACTCACCGAAGTCGTGTACCC[A/G]CCTCCTGCTGTGGCCAGCT |
| 03 | | 5 | TACCGCGTTGCTTCAC |
| MI01 | 1 | 16563334 | AGAATGGAATTTCCATTGTCATTGGTGCATTCCCA[C/T]TATTATGTTTACAAAAGGCTA |
| 04 | | 6 | GCATTAGTTGGAGA |
| MI01 | 1 | 16694341 | TGGACCTGCCACCTTTCGACGTTGCGGTTGTTCAA[A/T]TCCTTCTTGAGTGTCTCTGA |
| 05 | | 5 | TCCTGCAACCCGTTC |
| MI01 | 1 | 17018574 | TATGGGGGTTACTTAGAGTAGGAATTGGCAAGCTT[C/T]GGTGTATGTCAAACTCGATG |
| 06 | | 3 | ATGAATTTAAACATA |
| MI01 | 1 | 17189310 | TGGGGATATCCTCCTATGGGACAGTCATAAACGTC[A/G]CAGGGATTTGATCCATGGTC |
| 07 | | 5 | GACACAAGGATCATA |
| MI01 | 1 | 17324087 | ATCCAAGCCACCAGAACGCAAGGAGCCTATAACAA[A/C]TAGCTGTTAAATAAAAGAT |
| 08 | | 1 | ACAATACTGTATTAAA |
| MI01 | 1 | 17842856 | ATTTACTTGTGATGAAAATTCATCCAGCAGCACAG[A/C]ACTCAGCAGTTAATTAGTTA |
| 09 | | 2 | GTTCAACACTGACAG |
| MI01 | 1 | 17926581 | GAATCGGCGACAGTAAGAACCATGCCCGAAAAAAC[A/C]GAAACAAACATGACCATA |
| 10 | | 8 | TCAATAAGTGAAAAGCG |
| MI01 | 1 | 18042907 | TTAAGTTCACACAGAAGAAATGAAAGCTCAATATC[A/G]GCGGGCACTTACTGAAGG |
| 11 | | 7 | AATCCCAAGAAGCTTTT |
| MI01 | 1 | 18053800 | ATACACGAGACAAAAGTATGAATCGCGTGCATATC[C/T]ACGTTGTTGTCCTCACTATT |
| 12 | | 6 | GAATATTGACACCCC |
| MI01 | 1 | 18075963 | CTTGTATCTAAATGTTTGTAGTAGGGATACAAGCC[A/G]ACCACGAGAGAGAGTGGAT |
| 13 | | 6 | TTGAAGTCCCTAGTGG |
| MI01 | 1 | 18231938 | TATACCACATTAATTAATTCTAGGCATGTGTGTTT[G/T]GTTTCACATTATAAAGTGGGA |
| 14 | | 8 | TTTGTCAAACTTTG |
| MI01 | 1 | 18252481 | CAAAAATCCATAGATGTTTTTGACAATGGGGCATA[A/G]CCCCTATTTAGTTAGAAAAT |
| 15 | | 6 | AGGCAACATAGGTTC |
| MI01 | 1 | 18252490 | TGAACAAAGGCAACAAAAGTTAGCCAACATGCGGC[C/T]AGACGGAAAGGCTATGCT |
| 16 | | 0 | AGGCGTCCTAAACAATA |
| MI01 | 1 | 18494165 | AAGTTCAGTGCACTACGCTGACACTCCCTCCTCTC[C/T]CTGCGTGCACTGCTGCTAG |
| 17 | | 9 | TACCAGCAGCAATAAG |
| MI01 | 1 | 18743443 | AGAGAGAGAGCTCAAAGCTGTACCACTCGAGCTGG[A/G]AAAAAAAAGGTCCTCCA |
| 18 | | 6 | CCCAATAATCTTGGTGAG |
| MI01 | 1 | 18766967 | TTTATTATTAGAGTTTCGTGGCGGCGGTTTGGTTG[G/T]ATTGGTCCGCATTTATGAAGG |
| 19 | | 1 | AATGTTCTCCCCCT |
| MI01 | 1 | 18896497 | TTACCAAAATAACACACATGACCACATCTCAATCA[C/T]GTGTTCACAAAATTATTTTA |
| 20 | | 1 | GTTCCTAGAAATAAT |
| MI01 | 1 | 19012855 | ACTAGAAATCCATGTTGCTCACCGGCAAACATGTC[A/G]GGCGGCGTGAATTTGAGGC |
| 21 | | 1 | TCGGCGGGATCTCCCC |
| MI01 | 1 | 19056767 | GACTGAAGAGAGCAATGCAGAGATGATTCGCCTTC[A/G]CCCACTGCTTGATCCATCG |
| 22 | | 7 | TATCGTATCGTATCGG |
| MI01 | 1 | 19059513 | GGCAACCAAGCGTGTAGCAGCTCGGACGGGGGTCG[C/T]CCGTGGAGGGTCACCGGC |
| 23 | | 2 | GACTAGAAATAGCAGGC |
| MI01 | 1 | 19459096 | TTCTCTAAAATTTAGATTACAGGGCGCTTTAGATA[A/T]ACTGCTCGAGAGAGTCCTAC |
| 24 | | 4 | TGCAGCGCACTGCGC |
| MI01 | 1 | 19518089 | TGCACGCGCTGGAGCCGATGTGACTACAACCACGA[C/T]ACTTGGGCCTATCGCTGCG |
| 25 | | 0 | GATGTAGGACCTCTGT |
| MI01 | 1 | 19646555 | TACAGTTCACCACCTCTTTCTAACAATAACAAAAT[C/T]ATCTAAAACCTTGCCTCCTT |
| 26 | | 1 | GAACATGGACCGGAA |
| MI01 | 1 | 19680778 | ATGGTTCTGATGGGCCGAGCGTCCAACTGTATGAG[C/T]TGGTCGTCTAACTCAGACC |
| 27 | | 1 | AAGAGACGGTGGAGAA |
| MI01 | 1 | 20067468 | GAAAGGAGGCGTTCCACTCAGCTGAGATATGCATA[C/T]AGCAAATATTCCAAGCCCG |
| 28 | | 6 | AAAGTTCTGAAGAAAT |
| MI01 | 1 | 20203276 | ATACACATGGATCCGATATGGATTTGTAGATCTCC[A/G]CCCGCTTTCGACTTCGTATGT |
| 29 | | 1 | TACCGATTGTGATG |
| MI01 | 1 | 20204238 | GACAAGATGCTTCCATGAGTTAGGGACTACTACAT[A/C]GGGAAACAAAAGCAGATTA |
| 30 | | 6 | GTTCTCAGTCCGCTAA |
| MI01 | 1 | 20411305 | TTCTGACAGCGGCTAGGTTCAATTCGATCACTCTC[A/C]GGACGAAACGAAGCGGAC |
| 31 | | 2 | GTGTACAGAACATGACG |
| MI01 | 1 | 20429489 | TATATATGCGCGCGCAAACTGCCAACGACCTTTTC[C/T]CTTTGAGCGTGAGTGACAC |
| 32 | | 6 | AGATGTATCTGAATCA |
| MI01 | 1 | 20836291 | CGTCGCCGAGGCCAGGTGGTTCTGTAGGTCCAAAC[C/T]AGCTGCTGCAAGTCCACT |
| 33 | | 7 | CCCAGTCACCAAGTTCG |
| MI01 | 1 | 20913718 | ACATTGGGTTATAAGGTCAAGCTTTTTGGTCGACA[G/T]ATGGCATCATAAACATGCTA |
| 34 | | 6 | GAAAAAGCACAATAA |
| MI01 | 1 | 20957700 | AGAGAAGACCTCCACGACGGTTGTGGTGCAGCATG[G/T]CGGGAGTGCTGTTGCAGC |
| 35 | | 5 | TCACGATTGTCAGAGTG |
| MI01 | 1 | 21303040 | GTTCAAAAGTTTACTTATGCCAGGAATGAGTGCAA[C/T]AATATCACATAGCCTAAACT |
| 36 | | 4 | AATGCTATGTGATTG |
| MI01 | 1 | 21338048 | TTATTATCGATGCCTTAGGGGGATTGAAATCAAAT[A/C]CTCAACATTGGCACCCACCT |
| 37 | | 9 | CCAGTGATTCCACGA |
| MI01 | 1 | 21477819 | TCCATCGTGACCATATCAGTGGTGATGCCAAGATT[C/T]GAGCCGACTACTTCTGTGCA |
| 38 | | 9 | TAACCCGTGTACACG |
| MI01 | 1 | 21477842 | AGTCGATCTTTACTTCGTTCAGCGCCCAAATTGTG[C/T]TGGGGAGCTTGGTCTCTCTG |
| 39 | | 5 | CTCTGCAGAAGGTTG |
| MI01 | 1 | 21544929 | GATGCAATTGCTGCACTGAAGCGTTATATAATAAT[G/T]TTCTACTTGATGGAAAAGCA |
| 40 | | 7 | ATGAAGAAAGAAGTC |
| MI01 | 1 | 21558402 | ATAAAAGCTGCATCGGCAAGCCGAACACAAACCTA[C/T]GATGAAGAGAGCAGCTCA |
| 41 | | 5 | CCTGCAGAGGAGAAATG |
| MI01 | 1 | 21663234 | ACAGGACAAGTCGTTTTCGCCAACAATAAGAAATG[A/C]TTCTATCTAGTATCTAGAA |
| 42 | | 2 | GATTACATATCTAGAA |
| MI01 | 1 | 21767143 | TTACATCAGTTATTGTGTTTCAGGTGTACATATAA[C/T]ATTTCATTAATAGTTGTCTTC |
| 43 | | 8 | CTTCTTTTTTTGTA |
| MI01 | 1 | 21960166 | AGGGAGGATGGGCCAAGCGAAGAAGATGAGGGTCC[A/G]AGGAACCAAAAGCAAGG |
| 44 | | 0 | TGGATAAGATAGGGTGAG |
| MI01 | 1 | 22042897 | AATTTGCTTGGCTGTTGCTGCGAAGGTGATGAAAG[A/G]TTGCTTGTTGCAGAATACA |
| 45 | | 7 | TGCCCAATGATACGCT |
| MI01 | 1 | 22259850 | GTTCCAAGATGCTTTGACAGGGCAACATGACCATT[A/G]AATCGGCCGGGCCCACAA |
| 46 | | 5 | GTTCACCCAATTAGACC |
| MI01 | 1 | 22337106 | TCCCGGAGGCTCTGTAAAACTTTGAAACAAGGCGT[A/G]TAGATATCTAAAAACAAGA |
| 47 | | 9 | ATATCTAGTTAGATCG |
| MI01 | 1 | 22661685 | TGGTTAGGTGGTAGTACAAAAATGATATTCGATTT[C/T]AATGCTGTTGGGGGTACTGC |
| 48 | | 1 | TAGTGAATAGGACCG |
| MI01 | 1 | 22811688 | TAAGCTCCTTGGTTCTACAGTCCCTTGCCCTTCTT[A/G]ACCTCATAATCCAGATACATG |
| 49 | | 2 | TGTAAAAACAGCTT |
| MI01 | 1 | 22832121 | CGTCAGACCCTGAGCCATGGTCCAATGAGTACGGT[C/T]GGACTAGTATGATGCAACG |
| 50 | | 3 | AGGACCTCTCTAATTA |
| MI01 | 1 | 22938224 | GGATTCATGTCGTCACCCCATCACCTTCTAGTGGA[C/T]GCCAATCGACCCCCACTTCT |
| 51 | | 6 | CCTAGCTTTGGGCGA |
| MI01 | 1 | 23043652 | AGACATAGCGTAGCCTTAGCTTAGATCTATTTGTG[A/G]CCTAGCACAACCATCTCTCT |
| 52 | | 6 | TCGACTCTACGTCGA |
| MI01 | 1 | 23052497 | GCTTTCGCTTCCACTTCCTTGCAAGTTATTCTTTT[A/C]CCAGAGAACTTCAATGGCTG |
| 53 | | 5 | GAGGTAGAGGTGCTA |
| MI01 | 1 | 23053364 | TGACTTTTATACCATGTAGGAATATAATGGAGGGT[A/G]TAGTGATTCTTCATGAGACTA |
| 54 | | 3 | TTCATGAATTTCAT |
| MI01 | 1 | 23065458 | TCTAGGCTTCTACTTCTTGGTGTCTTAGATTTTTT[G/T]AATGTCTTCAAGGTCTTCAAC |
| 55 | | 9 | ATGGATACTAATGT |
| MI01 | 1 | 23102029 | AGTCATGTGGTATTCCCCCCAGTGATTATTCCCAT[A/G]GCATTCTCATAGATGGACTCT |
| 56 | | 0 | GCAAGCAAGGTAAA |
| MI01 | 1 | 23384675 | CACTCTTTATTGAGTATATCATCTATTGTCTCTCT[C/T]AGATGCTTGTTGTCATAGTTC |
| 57 | | 7 | GCAATTCCTATCTG |
| MI01 | 1 | 23752764 | CCGGGAATATCCAATCTAACGGAGTAAATACAACA[A/C]TGCGCGTACAGTAAACAGT |
| 58 | | 5 | GCGCACCAAATTACAC |
| MI01 | 1 | 23956652 | TGTCTAATTCTCTAGTGTGTGGGTCAGTACCAACT[G/T]GTTCGACTCTAGCATAGTTG |
| 59 | | 5 | ATATCGATCGGGCCA |
| MI01 | 1 | 23995691 | GAGCTCCAAAACATGGGACCAGCCAGGGTCGCAGT[G/T]GCACTCGTAGCCGGGAGG |
| 60 | | 6 | AAGAAGGGCCGGAGCCG |
| MI01 | 1 | 24020545 | TATTAGATGAGATAGGATAAATGCTACCTGCTGGC[G/T]CCTAGAGACAGCCTTAGCTA |
| 61 | | 8 | GCTCGAGCCGACTTT |
| MI01 | 1 | 24244933 | TCCTTAAGCGCCCAAACTCCATAGACTCATCGCCC[A/G]CCATAAGGTGAGCACGAGA |
| 62 | | 4 | TTCCGCTGGCGACGAT |
| MI01 | 1 | 24248021 | TTATAACTAGATCCAATATGTTTGAATATGCAGAA[A/G]TGTTTACTTGCCCTAGTTTTA |
| 63 | | 8 | CTCTAGGACATGTC |
| MI01 | 1 | 24556017 | TATTCAAAGACAAGTGATTCAATGGCTTCAAGGAC[A/T]GGGCTTCAATGGCTTCCAC |
| 64 | | 8 | GAACTCACCCTCCTTC |
| MI01 | 1 | 24678109 | ATAATTAGGAAGTGTGTGGCGACTCTTTTGTTGTT[C/T]GAAAAAGATGCTTGCTTTTT |
| 65 | | 7 | TCCTTGAGAGATGAA |
| MI01 | 1 | 24687067 | TGACTTGATTTTTGGCTTGAATGGCAAATGCTCTT[G/T]TGTAACTGCATGTGGTACAT |
| 66 | | 2 | CTGGACTGTGGATGG |
| MI01 | 1 | 24740071 | CCACTGAGATTGGGCTGGATCGGGTTAAATTGAGC[C/G]CAGTACTGTTGATACTTTTC |
| 67 | | 3 | TTTTTCCTTTTCTAC |
| MI01 | 1 | 24879985 | TCTAGGATATTCACTGGATTCACCTCATGCACATG[A/G]TCATGGATACTGAGCCACAG |
| 68 | | 8 | TTTTAAAGTCTTGAG |
| MI01 | 1 | 24892784 | TTGTGCAAAAAACGGCCACAGTGACACAGCCTTAT[A/G]TATAATCAAGCTGTGAGCT |
| 69 | | 9 | TGTGACTGTGTATTCT |
| MI01 | 1 | 24998236 | AGGGCAACGAAATGTTCACTGCCTTACAGCAGAGC[A/G]TGAAGGCTTCGAGAGCCA |
| 70 | | 4 | GCGCCCAGTCAAAGGCA |
| MI01 | 1 | 25150217 | CACTGCCATCACAGATTCACAGTGTGGCATGCACG[G/T]CAGCGTTTACTAGTCGTCT |
| 71 | | 2 | AAAGGTACTGACAAGT |
| MI01 | 1 | 25236202 | GGCGACGCCATCTTTTTGTGGTTTCCTTTTTTTTG[G/T]TTTGCTTAATGACGAATTGGG |
| 72 | | 1 | CTGGGCCGCTGGGC |
| MI01 | 1 | 25243804 | GCGGACCAGAAGAGTACTCCTTCGGTCCTTCCCTT[A/C]CTAATCCTTTAGTAGTAGAG |
| 73 | | 2 | AAGAGGACGATCGGG |
| MI01 | 1 | 25285220 | TTGGCGCTTGCACGCACTATTATATTGGTTGTACC[A/G]AGAGGGACTATAAGTCTTAT |
| 74 | | 1 | CAGATCACACTAACT |
| MI01 | 1 | 25395497 | CAATCATCAAGAGGCGAGGCGAACAAAGCTTTTCT[C/T]CGAGTATTAAAATATTCATC |
| 75 | | 7 | TGGGGCAGTGCTCGA |
| MI01 | 1 | 25395914 | ATCAAGCTGAGTAATACTGACAACCTGTACTAGTT[C/T]GGTAGTTGTAACTAGCACCT |
| 76 | | 5 | GAGCTACAAGGTCTG |
| MI01 | 1 | 25604637 | AAACATTTGAACCATAAAAGCCATAAGATAGCAAT[A/G]TTTCTTCCATATCACTTTCA |
| 77 | | 6 | CTTATTGCTTGAGTT |
| MI01 | 1 | 25642982 | TCTTAAGACCTTCTTATCCTCAACATCCATCTGTA[A/G]ATCAAATTCTTGAAGAAGGT |
| 78 | | 1 | CCCATAAGGAGAGAA |
| MI01 | 1 | 25933163 | GTTGTCCCCACGACAATCTAGTACAACCTTTGCTT[A/G]TATGTTGCACCTTTGACCCT |
| 79 | | 2 | GAGAAGTGGTGCCTC |
| MI01 | 1 | 25985986 | TCTATACTTAGATTCACATTCAAAATTTGTGTGAT[G/T]AGATTATACCATAACCCCAAA |
| 80 | | 1 | TTCTGTCCGACCAG |
| MI01 | 1 | 26247910 | ACACAAAATGCTGCCACAAATAAAATTTTGGAAAA[A/C]ACGCACACACTATGGTCAA |
| 81 | | 3 | GTATATGGAAAACAAG |
| MI01 | 1 | 26466258 | AGTCCGGCGCTGTTCTGTAAGTCGTCTAAGCATTG[C/T]GTTCACTGGCTCACTGCAG |
| 82 | | 2 | CTACTCTACACCAGCT |
| MI01 | 1 | 26538430 | TTCTCCTCCTCACCCTTCTTGATGGAGAGCGCCCT[A/G]CCGCGAGTTGCTTTCAGTAC |
| 83 | | 9 | AAGCATCGTAGCTCC |
| MI01 | 1 | 26632531 | AGTGAACATGTTGTTTTGAAAAGCTAACTCCATTC[A/C]TAAGGTACATTCATGTTTGA |
| 84 | | 0 | GTATATTAGAATTAG |
| MI01 | 1 | 26823700 | ATGGAGATACCATTATTGTACTAATTTATCAGGAA[A/C]AGTTGTAAATTGAACTGGTT |
| 85 | | 0 | GGTTTGGTGTGTCAT |
| MI01 | 1 | 26901083 | TCGTTTTTTTTGGGTGGTGGTCGCATAGGCTTGTG[A/G]CCTAAGACAAAGTAGACAA |
| 86 | | 6 | GCACGTCGGGTCGGAT |
| MI01 | 1 | 27057241 | CTCGGGGTGGAGTTCGAGTTTGTCCCATTGCTGCG[A/G]TCAGTGCACGAGCTGGATC |
| 87 | | 8 | GGTCGGATTTCTTGGT |
| MI01 | 1 | 27079746 | ACCTTACCTAGTCGGGCCGGGCCAATGTGTTGGGC[A/G]GGCTCGGGCTTGGGGCAA |
| 88 | | 8 | AAATTAAACCTGTGGGC |
| MI01 | 1 | 27174699 | ATGTTTGGTTCGGGAGCGAATAGAGTGGTTGGAGC[A/G]GAGCGACTCTAAATCGGG |
| 89 | | 1 | AATATTACCACTAGACT |
| MI01 | 1 | 27270910 | TTCTCCCTCTCTGATCCTCACCGAAGGTGCTCTCC[A/G]ATTGGGTCGTCGCGATGGTT |
| 90 | | 3 | CGATTTGATGGATGA |
| MI01 | 1 | 27271942 | GCCAGATCCGCTCGAGGCTGACCAGATTCAGGTCC[A/C]TCCGCAGTGGTGCTTCCAT |
| 91 | | 3 | TATTCATAGGCCTGCA |
| MI01 | 1 | 27309969 | TGTCGAGATATCTGTTTAGGTTTAGGGGTCGTTTG[A/G]AAATCGAGTTACTCAGATGG |
| 92 | | 9 | CAACTATATAGTGGT |
| MI01 | 1 | 27590086 | TGTCCCCAATCTGTAAAAAACAACGAGCACCCGGC[G/T]GTGGTGTTTGGTAGTACTT |
| 93 | | 9 | TTTTATACTGCTTGTA |
| MI01 | 1 | 27597724 | TGGTTGATGGAGACTGCTAGTGCCTGTATTGTATC[A/G]CCAATCATGTTTACTCTATAG |
| 94 | | 4 | TGTATACAAATCGT |
| MI01 | 1 | 28016175 | CCAGATACTCTACCTTTTTAAGTGTTTAACGCCTC[A/G]ATGATGACAAAATACAAGTA |
| 95 | | 5 | ATGGTGTCATAACCT |
| MI01 | 1 | 28089730 | TACTATTGCTTGAGTGTCGCCTTGTTCAACTTCTC[A/G]ACCAAGGCAGAGAGAGCTT |
| 96 | | 8 | TCATCCGGCAGCCGCT |
| MI01 | 1 | 28127457 | GCTGACTCTTCTTGAATTCCTTCCTGCTCATTTTC[C/T]TGTGCTGGCATTGCGAATCGC |
| 97 | | 4 | GCAACCGCAGGAAC |
| MI01 | 1 | 28146988 | TTTTTGCATAGTTTGATCGCTAATGCTGGTAATGT[A/C]CAGGTCGATGTGAAGAGGGC |
| 98 | | 1 | AGAAAAGAAAGAAAC |
| MI01 | 1 | 28396767 | GGTGTCATTGTTGATCCATAACTCACGCTTATTTA[C/T]GAAGCAAGAGTGACATTTCT |
| 99 | | 1 | TCGGATCCATAACTA |
| MI02 | 1 | 28521057 | TCGATAACGATGTGGCAAGTCTCCCCCGGAGCTTT[A/G]GGGCCGACCAGACTCTCCA |
| 00 | | 1 | CCGAGACAATGGTGGC |
| MI02 | 1 | 28529955 | GATTGAAGACGACCTGCATGCGGATGAGACCATAG[A/C]TATTTTCGTAGGAAACAAT |
| 01 | | 2 | TACAACACCTACACTT |
| MI02 | 1 | 28541068 | AACTCATGAACACGAACTGCTCTGCTGAGCACACT[A/C]AAACCAAAACTAGATTATT |
| 02 | | 1 | GGTTCGTCATTATTAC |
| MI02 | 1 | 29029741 | GCAATTCTACTTGAGGTTATTGGTTAATCATTATC[A/G]TATCACGTGGCATCCAATGTT |
| 03 | | 8 | AATGTTCATCTTGG |
| MI02 | 1 | 29089129 | CAAATTCAAGCTGGGCTATGATTGGGCCTCTCTCC[C/T]TGCTAACATGATGGGCTCGG |
| 04 | | 0 | CTCATCTAGCACAGG |
| MI02 | 1 | 29089165 | GAATAGAATCCCTAGTTCGTCCCGGCGCGCGCGTC[A/G]CGATCCCGACACCAAGCTG |
| 05 | | 6 | GGCAAAAACCAAAACT |
| MI02 | 1 | 29168788 | ACTACTTAAATTTTTACTTGTGGAAACGGCATACA[G/T]GCAGTTAATTCTGGATTCTA |
| 06 | | 6 | GCATTGGAGAATTGG |
| MI02 | 1 | 29398351 | GCGGCGATGACGAGGCAAGCACGACGATAAGATCG[A/G]TGGGGATGGCGATGTTCC |
| 07 | | 2 | GGCAGGACGAACCCGAA |
| MI02 | 1 | 29457938 | GTAAAATTTAAGGATTGGATCATATTTGGATCTTG[A/C]TCTATTTCTATTCATTCTTGA |
| 08 | | 8 | ACTAAAATTAATTA |
| MI02 | 1 | 29462596 | AAGGACGGGGTGCGGCATCAAGGCCGGGAAGCACG[A/G]GCCACGCAGGAGGACAA |
| 09 | | 2 | CAGATGTAAGACCCAAAA |
| MI02 | 1 | 29570871 | ACACTCTTTCTCTTAGTGGAAAGACCCAGATGATG[A/C]GGGCTTATTAAGGCTTTACT |
| 10 | | 5 | TACAAAGGCCAAAGG |
| MI02 | 1 | 29595632 | CGTGGCGTGCCGGAGAACCGGTTCTACAACCCTCC[C/T]CACGTACGTCGACAGCAG |
| 11 | | 0 | CAGCAGGAGCAACAGCG |
| MI02 | 1 | 29678760 | GTTTGCACCAGCAGATATGTTCTCCACATGAGACC[C/T]AGAGTGAAAAAAAATCACT |
| 12 | | 5 | AAAACATTAACTACCC |
| MI02 | 1 | 29927618 | CTGGCTAAAGCAATGCAAAGTCCTGGTTTCCTAGC[A/G]CAGTTTGTACAGCAAAATG |
| 13 | | 8 | AGAAGAGCAGAAGAAG |
| MI02 | 1 | 29949764 | CGTCTCTTAACATCTCCCCGTTGATCTTCTCCGTT[C/T]TTCTGCAGTGATGTGACTGA |
| 14 | | 6 | AGAGATGGCAAGAAG |
| MI02 | 1 | 29983020 | CATTACACGCTAGAGGCCCTTTCGCAGCTAATGTT[A/G]GATGTAGTGTACTGGAATTC |
| 15 | | 7 | TAATTTCATCTCATG |
| MI02 | 1 | 30021241 | CCCTCAGCAAAAGTACGCTTGTCTACTGCAATAAC[A/G]TCAACGTCGTCTACCCTCT |
| 16 | | 9 | CCATCAACCCCATCTA |
| MI02 | 1 | 30050229 | CCCCATGAAACGGCAAAGCTTTCGGCTTTCGCCTT[A/C]ACCTCGTATGCGTTGCCAA |
| 17 | | 9 | GCCTGGCTTGGTTAAC |
| MI02 | 1 | 30051593 | TTGCTAGGCATTTCTGGTTTCGTTTGCTCGGTCAG[A/G]TTAATTTGCAGGGATTCAGC |
| 18 | | 9 | CCCCAACCAGGTGAA |
| MI02 | 2 | 750292 | GTGGAACCAATCAACCCTAGCCATGTAGAACCTAA[A/G]CATCAGAGTCTTTACAGTA |
| 19 | | | ACCCAAAATTGGGCGG |
| MI02 | 2 | 2372065 | GAACTAAGGAGCTACAGCAGGGGCTGTTAAGAAAA[A/G]TATATCCTTAGCCCCTGCA |
| 20 | | | AGTTCCCAAAAAATGC |
| MI02 | 2 | 3233845 | ATCCCCTTGCCACGCTCGCCATCGCGGTGTTCTTG[A/G]TCTGCCTTCTTCTCCCCACT |
| 21 | | | TCGTCTTCTTCGTCA |
| MI02 | 2 | 3379765 | AGAAGTTGAGTGTGGGAGGGGGAGGGTTGTGGTTC[A/G]ATATGGGAGGGATGTTGG |
| 22 | | | AGGAAGACACATGAAGA |
| MI02 | 2 | 3754733 | ATACTCCTCCTGCTAGATGTGAACCTGCAACGGAC[A/G]GTGATAGTAGAACAAGCGA |
| 23 | | | GAGTGCCGCCGCTCTC |
| MI02 | 2 | 5111263 | ACACAGCACAACAGCTGACCAGACAAACCACTGCG[C/T]CATTCAAACCAGCGCCTG |
| 24 | | | CCGATTAAAGAGGCCAA |
| MI02 | 2 | 5276017 | TATCCACATGAGCAGAATAAAAGAGTCGGGGATTT[A/G]TATCTTCTACGGTTGTACCT |
| 25 | | | GAAAGGTCAACGCTC |
| MI02 | 2 | 6468284 | AGAGACTAATAGTAGACAGACAGTAATACAGTATA[C/T]GTACATACATAGAGTCAACT |
| 26 | | | ATTAACAGTAAGACA |
| MI02 | 2 | 6986622 | CGAAACACTAGTTGAATGAGGATCCGATATTCTTT[A/C]TGAAAGATGGGCTGGGCAC |
| 27 | | | ATTTGAACTGACTGAA |
| MI02 | 2 | 7185307 | TGAGAGCTCTGAAGTAGCAGAAGAACATATATTAT[A/G]GGATCATCTCATCTGTACAG |
| 28 | | | ATGTCAATAATCTGC |
| MI02 | 2 | 7270898 | CATGCCAGTGCCCTGATGGGTTTGAGCTCCTCAAC[A/G]GTCTAAATTTCTCTAGAGG |
| 29 | | | ATGCCGGAGAAAGGAA |
| MI02 | 2 | 7329531 | TGCTAGGGAAACACCAAACAGGGAATGTTCAGAAC[A/C]ATCTACTATGCTTGAATCC |
| 30 | | | TCCCAACGAATTTGGG |
| MI02 | 2 | 8914166 | CTCTGAAGAAAAGGGAGAGGAAAGACAAGAAGAAG[A/G]CTGCAGATGCTGAGAAG |
| 31 | | | GCAATTGAGACCGCACCC |
| MI02 | 2 | 9452388 | TTGGGAATTTTTTCGGAGCAACGCGAACCAGGCAG[A/G]ACCGCAGAGAGCACGAGT |
| 32 | | | GAAAGGAGTGTAGACTT |
| MI02 | 2 | 10148921 | AAGAAGCTGAGATCCATTATAAGCAAAAGGTGTTT[C/T]TTTCTCTGAGGTTTCCATTT |
| 33 | | | TTGGGCTCTGTGCTA |
| MI02 | 2 | 10492681 | CTCGGTCTAAAGTAAGCATGTGGCTTCGTTCCCTT[A/G]GATCTGGTTCAGGACTTTTG |
| 34 | | | GTTTGTTTCTTTGGG |
| MI02 | 2 | 13321078 | TCATGGCAGATGCTCCCTTCCATAGCTTTGAGCTC[C/T]GTAGACATGCACGCTAAGGT |
| 35 | | | ATCCCCAATGTGGGT |
| MI02 | 2 | 14343191 | TCTTTGGAAGGTGTATTTTCCCTTGGAAGAGCAGG[A/G]AATTTGACTGCCGAATCGT |
| 36 | | | CTGCCGGAAAAAGTGG |
| MI02 | 2 | 14472563 | TATTTCTAGGTGCGAAAACTACTGGCAAGGACCAA[A/G]GAGACCGTTTGCACCAGAT |
| 37 | | | TGGATCATAAAGAACA |
| MI02 | 2 | 14902322 | CTTTCTTCTCGGGAGCGTCCATTGGAATGTACGGC[G/T]GCGGCTCAAGGAGCATTCA |
| 38 | | | CGAGGAAATCTATGTA |
| MI02 | 2 | 14903218 | CGACGACGACGACGGCCGTCGCTATTGCCGTGGGA[A/C]TCGCTCTGCACTTTCTTCC |
| 39 | | | GGCCATAGCTAGTAGT |
| MI02 | 2 | 15866437 | ATATCTCCCTTGTGTGTGTCGAGTATGCGTGCCTC[A/G]TGCTGAAGTTGGATGCCATT |
| 40 | | | TTTTTAGAACATAAA |
| MI02 | 2 | 17557757 | AGCTTTTTTTTTAATGGCGAATATGATCGAGATAT[A/G]GTGGCAGCCCCATTTGGAGA |
| 41 | | | GAAGGGGGCCCTCCA |
| MI02 | 2 | 17959632 | GTAGCAGCAGCAGTACGTGGATTGGCTGTTGATAT[A/G]TATACGACATGGCATCCTAG |
| 42 | | | ATTTAAAATGTCTGG |
| MI02 | 2 | 22041965 | GCCAAGATAGTGGTGCCAACCAACTCACCCCGTCA[A/C]CCAAAAAAAATGCATGCC |
| 43 | | | AAATTCTAGAGAGAGTA |
| MI02 | 2 | 22128902 | CGTACTCCACTATTGCTCCCAGCAACTCCCAAGCG[C/T]GCAAGAAGCAACAAGTGA |
| 44 | | | GTTATGGTGTGAGGCCA |
| MI02 | 2 | 22441022 | TAACGGAACGGTACGGATACAGACGTGCAAACGGA[C/T]AAGCCTTTGTTTTTGTTAT |
| 45 | | | CCGGGGCTTCCAATAT |
| MI02 | 2 | 22659023 | TGGTAATCGTCAATGGCACCTTGACAAAACATTTT[A/C]TTATCCTCCAGGTTCCTTTAT |
| 46 | | | TCATGTTTACTAAT |
| MI02 | 2 | 23235700 | CTGTGACAAACGTTGCCTTACTGGCATAGGGTTAC[A/G]CAGTATATATTCTGTAGCAA |
| 47 | | | AAAGAAATATGTTCA |
| MI02 | 2 | 23717407 | GAAACGACCGCACTACGAGGCCACCTAACAACGGC[C/T]GCAGCGGCACTAGAGAAC |
| 48 | | | AAACGAGCAATCTGTTT |
| MI02 | 2 | 27476344 | GGGGGGAGAGAGAAGGGAGCATGAAACAGGTAACA[A/G]CATTCTAGATTATCCAAC |
| 49 | | | GATGGTTTAGAATTTTG |
| MI02 | 2 | 29302232 | ACCGCGCACGACGCGATACAGTCCAGAAGCCATGT[A/G]TGAGGAATGGCTTTGCAG |
| 50 | | | CCAGCCGTGGTCGCCAG |
| MI02 | 2 | 30944169 | CTTGACATTACTTGTTTCCTTAGAAGAGGTCTCTG[A/G]AAATTTGGGACATTGTTGTT |
| 51 | | | ATGTGAATCTCTGTG |
| MI02 | 2 | 32771740 | CTACACCAGCCTGAATAAGGCTTATCCCAAGGACT[C/T]GTATCCACTTCCACGCATAG |
| 52 | | | ATCAAATCGTGGACT |
| MI02 | 2 | 34162472 | TGACCCATGCGTCAAAAGTTCATCATAAAACGTGG[A/G]ACAGCTGTACGTTCCCGCG |
| 53 | | | TGCAATGGTGGACATC |
| MI02 | 2 | 34990440 | TGCTAGGAACGAGAGCTTTCCTCTAAGGAAGCTAG[A/G]AACGAGAGCTTTCCTCTCT |
| 54 | | | CTTTCTTACACTTTTC |
| MI02 | 2 | 35168369 | AGTAGGATACACTAACTGGTTGCAATTATTCCAGT[C/T]TGGTTTCGCGTTTATTTTTCA |
| 55 | | | CTGTTTGATTGATG |
| MI02 | 2 | 38471467 | ATGACTCCTTCGCCTGTCGAAGCAAACTGAAAACG[C/T]GAAACGCTCGCGAAGTCG |
| 56 | | | CGATAAAATGAACCCCT |
| MI02 | 2 | 38490733 | GCACCGTCGAAACGGCAGTGAAATTGCAAAAGTAC[C/T]TGTAAGCGAGCCTGATATC |
| 57 | | | TGACTGGCGGTGTTCT |
| MI02 | 2 | 39331612 | ATCACGTAATCTAAAACAGAGTGAGAAATAAAAAA[G/T]ATAAAGAAGTAAAGCACA |
| 58 | | | CAACCATCGAACCATGT |
| MI02 | 2 | 46524713 | TAGGAGTAAGTACTGTCCGAGCTAAACCCTCAGAT[A/G]GTACGTAGTACAAACACCA |
| 59 | | | AAAACTACACAGTTCC |
| MI02 | 2 | 47306731 | GGGAATCCAATCAGCGTGACAATCTGCATGGTGGC[A/G]TCCTACCACACTACCCCTG |
| 60 | | | GGTTCAAAAAAGAGTC |
| MI02 | 2 | 49447363 | ACCCCGTGACCGTACCATACGTTCACAACATAACT[G/T]TCAATACAAATATAACATAC |
| 61 | | | AATACACAAGACATG |
| MI02 | 2 | 51438880 | TTCAGTTGAAATAATACTGAGGAACAAGTCCTCCA[A/T]CAGGTGCCACATATTTACAC |
| 62 | | | GATTTACTCTTGACG |
| MI02 | 2 | 51442523 | TGATAGCTGCGCGCCTTTGAAAACATCAGTTCTAA[A/C]AGTAGTGAAGGAGTTTAGG |
| 63 | | | GCAATATAAACAACTG |
| MI02 | 2 | 51739192 | CCCCAAACAAATCAGATGTATGGCCAAAATGATTC[C/T]ATAAACCAGGATAATGGAAT |
| 64 | | | CCAGAGTGAGTATGA |
| MI02 | 2 | 51863660 | CTCGCCAGTCAGTTCGTTACGCAATTGCAGACTAG[C/T]GCTTTCCACTTTCAAGCCA |
| 65 | | | AGCTGCTGTCCTGTCC |
| MI02 | 2 | 53231096 | AGTGTGGACTCGCGAGAGCAAGAGTTCTCCATATG[C/T]TTGTTTGTTCATGCAATGCT |
| 66 | | | TTGTTTTGTATGATA |
| MI02 | 2 | 53728024 | GGTACATCAAGTTTAATCAGTCTATCAGAAAGTTC[A/G]GTTTTGAGGAAAACAAGGA |
| 67 | | | AGATGATTGCATTTAT |
| MI02 | 2 | 54151723 | ACAGAAGAGAAACCAACATAACTGCACGCATTTGT[G/T]TTGGCATGCTTGTGTTGTG |
| 68 | | | GACCATAAAACTTAGT |
| MI02 | 2 | 55758080 | GCTACTTTTATATATTAACTAGTACAATGCTCGCA[C/T]GCTAATCATATTACAATCATA |
| 69 | | | TTTGATGATAAAGT |
| MI02 | 2 | 55967207 | GTAGATTTATAAATTAGATAGGAGGTTGCCTCAAC[C/T]GTTCCTCTCTTCGGGTTTCA |
| 70 | | | ACTCCAAGTCTTATG |
| MI02 | 2 | 56308583 | ATAGCTATTTGCATAGACTACATGATTCAGTGAGA[A/G]GTGTACTTGAGTGGCGGCGC |
| 71 | | | ATCAGTCGCCGCCCT |
| MI02 | 2 | 59981369 | TTAAAGATATAAGATTATATATAATGTAGTAAAAG[A/G]TCATGTGCCCATAATTCTGTT |
| 72 | | | AGGCTTAGGATCAT |
| MI02 | 2 | 60557965 | TCGATGCTCTTAGATGCATATGACCTAAAAGTACG[C/T]AAAAACATAGTGAAAAATAA |
| 73 | | | AAAAAGACTATTGTT |
| MI02 | 2 | 62743981 | CAATTACAATGCTTTTCATGACCCCATTTGGTTTT[C/G]CTTTCTGTTTTCAAACAACTC |
| 74 | | | TGAGATTTGGCTCA |
| MI02 | 2 | 63605789 | GGCACACTACTAGTAGGTCAAGTTAAGACTCCACT[A/G]TCAATTTGCTTGTTAATGGT |
| 75 | | | TGCATCTTTGGAGAG |
| MI02 | 2 | 68923211 | GCCTACCTGCGCGGGCGATTACTGGTGTACGTGTA[C/T]ACATATCCCAATGGGACGG |
| 76 | | | GCCTGCTGGGTGGCCT |
| MI02 | 2 | 71991688 | CGCACAGTACTTGTACTAGCTGCTATCAGGTCAGG[A/G]ACTCTCAGGACTGAAACCA |
| 77 | | | CAGTGCAGGGAACCCA |
| MI02 | 2 | 78860220 | GGATGTAGCTGCGGCGACAGCACGGCCCACGATTT[A/C]TCTTCCTTCACCAGTGACT |
| 78 | | | CTGCTGCTGAGCTGGT |
| MI02 | 2 | 80044237 | TGGTGACCTGCTAGGCCTAATGAAACCCTTAGCCA[A/G]CAGATCGTCCAATTGTCGC |
| 79 | | | TTCAATTCCACCTGCT |
| MI02 | 2 | 81348249 | CGCCGTTCGAAGTCCCACCGCAGAGGGGGTTGTGG[C/T]AGCAGGGTCTGTGTTCAG |
| 80 | | | CCAATCGGTGCGACCTA |
| MI02 | 2 | 85197765 | TGTAAACATGCTCCAGCGAGGTGTTCCATCTGGTC[A/G]TGCAATACCACATTCCGGAT |
| 81 | | | TGTCAAGATAGTATA |
| MI02 | 2 | 87532481 | TATTCAGTGTAATGCCCATCATATTGAAACAGCCC[A/G]GAGTATTCTGCCTTGCAATG |
| 82 | | | TGATATCCTTCCCGT |
| MI02 | 2 | 88349291 | TGCAATGGATGAGGAATTGAGGTAGGGAAGAAAAG[G/T]TAACACCTAGGTCTAGTTT |
| 83 | | | GGGAACCACAAAACTG |
| MI02 | 2 | 88797128 | ACAAAAGAGAAGCTATACGGGTTGAATTAGCTCTC[G/T]TGCTATTAGCAGGCTTTTTT |
| 84 | | | AGAGAAGTACTACAT |
| MI02 | 2 | 89435062 | TTCAAGTTCTGCAGCCTCTTTAGCTTTCTTAGCTT[C/T]TTCCTTAGCCCAATGAGCTTG |
| 85 | | | AGAAAAACGTAAGA |
| MI02 | 2 | 89844641 | ACATGGATGGGAATGATATTCCAAGATCCTGGAAC[A/G]CAGACAAACTTCAGAGATA |
| 86 | | | CTATGTGTAATAGCTA |
| MI02 | 2 | 89855448 | CTGCTTTGTATACGAAGTCCTTCGGCGCCAAGGTT[A/G]TCTGCTTCGATACACACATT |
| 87 | | | CACAACGCTCGTACG |
| MI02 | 2 | 92149836 | CTCCTGCAGCATAGCCTTCGTATGAGTCAGTACCA[A/G]CTCCAGCTTCTACCGCAGAT |
| 88 | | | CCTGGATCCGAGACT |
| MI02 | 2 | 10398590 | CGACCGTCAATGGTGGCAGCGGGGTCTCACTCTCG[C/T]CTCGCCTCTCTGGTGCTCA |
| 89 | | 7 | ATAATGGCATCCCCGC |
| MI02 | 2 | 10634867 | AGCCTATCAACAAGTTTACACCTTGCCCTCATTTC[A/G]AAGCAGCATTAGGTGGGGG |
| 90 | | 9 | CCTCATCGCTTGCACT |
| MI02 | 2 | 10646199 | CTGTCTTTGCCCATTCTGCTGGCCCAATGGACGCC[A/G]AAGATTGGCTACAAACTGC |
| 91 | | 0 | GGAACGTGAGTTGCAC |
| MI02 | 2 | 10677322 | TTTAATGCGAAATAAATCCAAGACTCGGATCTACT[G/T]TTGAGAGGACCGCGGGTTG |
| 92 | | 8 | ATTTCAATGTTTGGCG |
| MI02 | 2 | 10824884 | TTGGCTAGCGCACAACTCGGCCTGAACGGTTTCAA[C/T]GGCATTGACGAATTGCTCG |
| 93 | | 7 | GGGAAATCCCAAGAAT |
| MI02 | 2 | 10983025 | TCGGGACGACATTCTCCAACAAGCAAAGAAAGCGG[C/T]CCACCGATTGCCCCATGC |
| 94 | | 4 | TCTCCCTCAACATCTGG |
| MI02 | 2 | 10996852 | AAAGAGGGAAAAGAAAGACAGAAAGCAAGGTCAGT[C/T]CCTCTAAATTCACGGCA |
| 95 | | 4 | GCAACAGAGTGATCGTGC |
| MI02 | 2 | 11050867 | TGAAACACGTAGGCTGTTTCAAATTTTTGGGAATC[A/G]GATATCAGCATTCTAATGCG |
| 96 | | 9 | GGTTAAGATTTTTTT |
| MI02 | 2 | 11079496 | CACCGCAACCATGCTACTGATCCCACGTCGTTGTC[A/G]CTTACACACGCTAGGTTTTT |
| 97 | | 8 | TTTCTTTTTCCTAGT |
| MI02 | 2 | 12027466 | AATTCAACCACCCCTACGCGTACCAGGAAAGATAG[C/T]GAATGGAATTAGATCATCC |
| 98 | | 5 | GGATGCAATTTGTTAC |
| MI02 | 2 | 12254049 | CGAGTATGGGCTCGTAGGTAGTCGTGGAGTAAGTA[C/T]GAAGGAAGTGCTTGCCATT |
| 99 | | 4 | TTCTTGTGGGCATACG |
| MI03 | 2 | 12307914 | ATAGAGGAGGGGTAGCGCCGTCGTGGGTGGAATCA[G/T]GGAGCACCGCCATTTGAG |
| 00 | | 2 | CTCGAACCCATGGACAG |
| MI03 | 2 | 12551151 | GTCCATGGCGGCTCTCCCTCGGCGTACGCAGAACA[C/G]TGGCTGGCTCTTAGCTTCC |
| 01 | | 1 | TTGTGTTCTATAGAGG |
| MI03 | 2 | 12622529 | GGCCCCACATGTATGCGAGAACGTGGTTGAAAGCG[A/G]TCGGAATGATGTGGCTGG |
| 02 | | 6 | CATGTGGGCCCTCGATG |
| MI03 | 2 | 12838605 | CAGGATTAGGAAACGATTTGTTCTTACCATACTTT[A/G]CAAAAGAAGAGAATAATTTT |
| 03 | | 1 | CCTTTCAAATGTGCC |
| MI03 | 2 | 13203583 | TCAATATCTATCTATAACCTATTAAAACATGGATG[C/T]TAGCCTTTTAGAACAACAGTT |
| 04 | | 1 | AAAAGCTATCAAAT |
| MI03 | 2 | 13253975 | AGTACATGAAGGATTTTATGGTACTTATCAATTGG[C/T]CCCAAAGATGAAGTGGCTTT |
| 05 | | 9 | TGAGGAGAGCTGGCT |
| MI03 | 2 | 13271971 | TTTTCTTCTGCATCTGATGCACGAAGAGATAGGAA[C/T]GATAGGAATGTGAGCGATTT |
| 06 | | 1 | ATAGAGGTATTAGTT |
| MI03 | 2 | 13602965 | CAACCATGGAAGCCGAGCTTGCTGCACTTGACACG[A/G]CAAATGTTGAGACAAAAT |
| 07 | | 2 | GGTTGCGTGAACTCTTG |
| MI03 | 2 | 13640991 | AAATTTACGAGTGAAGAGTGTAGAGTATGCTTTCC[C/T]TATTTCAAAGCCATTTTTGA |
| 08 | | 0 | GAAGAAAATCCTTAA |
| MI03 | 2 | 13693596 | TCCATAAGCTTTGCTTATTACAACAAAATTCACCC[C/T]AGACTAGAGGCAAGGCTAA |
| 09 | | 4 | CAAAGTAGTTCAACAA |
| MI03 | 2 | 13725761 | CTTGAGCATAGCTGAGTGTTGTTTCAGATGTTTTT[G/T]AGCCAAGAGGCGTAGACTG |
| 10 | | 8 | TGACAACGACGATATT |
| MI03 | 2 | 13835069 | CTTCTTTGGCCCGCTGCCCCTTGTCAGCACTACTC[C/T]AACTACGCTGCCTGATGCAC |
| 11 | | 8 | ATGCACCAAGTGAGT |
| MI03 | 2 | 14150048 | AAGTTCAATTCAGCATAGTCATTTTTGGAAACACA[A/G]CTATGAGTGAACCAAACTA |
| 12 | | 8 | TAATCAGACAAACTAA |
| MI03 | 2 | 14997259 | TTCCTCAGCCCAGCCCATGCCGCCCCTTTCGTCCC[C/T]GCTCCTGCTCCATTTTCCGC |
| 13 | | 1 | GCATCGCTTTCAGAA |
| MI03 | 2 | 15086513 | GATCCCTATCAGCTCATCAACATTCTAGGTGCTTT[C/T]CTATAGGTTATGGATTGTAAA |
| 14 | | 3 | TAAGAAGAAAAAAC |
| MI03 | 2 | 15354940 | CACATTACTTTTGTCTGAAGCTGGGGGTTCACCCA[A/C]ATTCGTAGCAAATATACAAT |
| 15 | | 0 | GTCCGATTATGATGC |
| MI03 | 2 | 15449121 | TCAGTTTCCCTAAAAACATGGTGAGTAAAATTAAA[G/T]TCATGCTTGTGTTAAAGTTT |
| 16 | | 5 | GGCAAGAAATTGAGT |
| MI03 | 2 | 15516481 | TGTTAAGAAGTTTTGGGCCTACTTCGGGCACCCAA[G/T]TCCAAGGTACCCAACATAT |
| 17 | | 4 | GTCGTATTCTAATTTA |
| MI03 | 2 | 15588080 | GCTACTGTCTCCGCCACCTACGTCAGCTTGCCCCT[G/T]CCTCAACGTCTATCAGCTGT |
| 18 | | 7 | CGGCCTCCAGTGCAT |
| MI03 | 2 | 15854726 | AAGCCCGTGGCTTACAAAGCGTGCGCAGGTGTTCG[A/G]CAGAACGCTTACACAAAG |
| 19 | | 5 | CTTGCCCCGACGTGGGC |
| MI03 | 2 | 16552702 | GTCGACTTGTACTTATGCTGGTGTGCTGCGTAGTA[C/T]AGGTACAGCATCATCCAGCA |
| 20 | | 3 | CGTCAGGTCAGTGTC |
| MI03 | 2 | 16918243 | TTTTTTAAAAAATTTCGAGTGACTGAAGCACTGGA[C/T]AAATTAGTTGTGTTCGGTTA |
| 21 | | 8 | CTGTGTAACATAGAT |
| MI03 | 2 | 17112869 | ACTTCTTATTAGCTACCCTTTTTTAGATGTTAGTT[A/G]ATTTCATCTTCCATAAAAGTA |
| 22 | | 3 | GTTGGCGATAGTAG |
| MI03 | 2 | 17238964 | TTGTCATTGGCGACCACAATTTTCGACATTAGAAA[A/G]GAATGTTTTTTTTGCTACAT |
| 23 | | 5 | CTCTTCCATAGAAGC |
| MI03 | 2 | 17289354 | CTCTATTAATAGCTGCAGCTTTACATTAAATCACC[A/G]GTATCAAGCCACCTACCCTTC |
| 24 | | 2 | TTGTATGAAGACGA |
| MI03 | 2 | 17414143 | TTTAGTGATCTCATATAGTCTTAGGGTGGAAAAGA[G/T]ATTTGAGCAAACTATAGGAT |
| 25 | | 5 | GAAATCCTAACTTGG |
| MI03 | 2 | 17415775 | TTCTATATATTTATATATTCTACAACAACATAGTG[G/T]TATTATATGTTTATAGTGTAC |
| 26 | | 6 | AACAACAGTAAGGC |
| MI03 | 2 | 17447504 | ATCTAATTAATGAAGAAGCAAGCCTCTAGACGACG[A/G]CCATGTCGCTGGGTTCTTC |
| 27 | | 1 | GCTGGCGCCGTTGGCG |
| MI03 | 2 | 17548419 | CGATAGATGATGTTGTCTTAAAAGCTACTTTGAAG[C/T]GTACAACTTCATTATTCGATA |
| 28 | | 1 | TCCCTCCCTACATA |
| MI03 | 2 | 17562425 | TCAAACTACATGATTGATATTAATAAAGCTTGAGC[A/G]GCTAAACTATGCCTGGATGT |
| 29 | | 1 | TGGGTGGGGGCAGGG |
| MI03 | 2 | 17700209 | GTTGCAGAAACGGTGGGTGGTGGGTGGATGCTTGT[C/G]GTAGCGGAGAATTGGAAG |
| 30 | | 3 | TTGATGTCCCTCCCTCA |
| MI03 | 2 | 17765851 | AGTAGACGAGGACAGTAATGTGGATGAGGAGGCAC[A/G]CCCGTGTAGGCGACGATA |
| 31 | | 8 | GCATCATGAGCCTGGTT |
| MI03 | 2 | 17852278 | AAGGAGCAATTGTCTCTATCAGGCAGACTGGCTTT[A/C]TTCGCCCTTTGCTTTTTCTG |
| 32 | | 2 | ATAGATAAAAGTTGG |
| MI03 | 2 | 17882320 | TATGTTTCCCATAATCACATGGCATTTGGAATTCC[A/G]GTTGTCTAATCCTCAGCTACC |
| 33 | | 1 | ACTCAGGAACACCC |
| MI03 | 2 | 17918909 | TTCTTTAAACGTGTCGTGTTCACCTTGTCGGGTCT[A/G]AAAAGGCTTGTATTGGGTCG |
| 34 | | 7 | AATTGATAAGTCTCG |
| MI03 | 2 | 17963325 | TTTCTAGATTCATGTCGTAGATAAACAATTAGCAG[A/G]GTTAGCTTTTAAATGAGTTAT |
| 35 | | 7 | GCTCATACTTAAAA |
| MI03 | 2 | 18048069 | TCCTTTCCGAGTTTTCATCAGTTTATTCAAAAGGT[G/T]GTACCATTGTCATACAACATA |
| 36 | | 8 | CATAGTTTGTTCAT |
| MI03 | 2 | 18131497 | GTATAGTGCGCACTTATGCAACTTAGCACAAGTAG[C/T]TCAGAGAAAACTCAAGAAT |
| 37 | | 5 | CATGTGGAAGCAACCC |
| MI03 | 2 | 18384684 | CACGACAGCAATCCGCGGCACATGCCGATCGACGC[C/T]CGCTGCTCCGTTCTACAAA |
| 38 | | 0 | TACACCTCAACCCACT |
| MI03 | 2 | 18622781 | TTGCAAAACTTATCGGCTGTTCATCGATGTCCCTT[G/T]GCTGGGTCCAGCCCTGGTGC |
| 39 | | 1 | ACACGATGCCTGGCT |
| MI03 | 2 | 18633994 | ATTGCGGCATTAAACAAGGGAAAGGAAAGGGCGGC[A/C]GGGTGGATCAGAGCACA |
| 40 | | 8 | ACTCCCGAGGGCAGAGCT |
| MI03 | 2 | 18708550 | CCCTCGTCTTGCTCGTCGGCGTCGCCGATGCCCTG[C/T]CCGGTGAACCTTCCATCATC |
| 41 | | 5 | ACTTCCGCCACTGCT |
| MI03 | 2 | 18880291 | TCCACCAACAACCAGCCACAATTCATTCAGATTTC[A/G]AACAGAACAATTGTCTAGA |
| 42 | | 5 | CTTACTATTTAGAAAC |
| MI03 | 2 | 18973166 | ACCCCCTTTTTCTTACTGCCATGTGCACCCTAAGA[C/T]CCCTCTTTGTTTTTAACCAG |
| 43 | | 7 | CAGTCCAGCAGGCCT |
| MI03 | 2 | 19067421 | AAACAAGTTTCTTGCAAAAGAAGCCCTAGTTCGCG[A/G]CCTATCTTTTGGGGTTTATT |
| 44 | | 2 | GATCTGGTGGAGGAA |
| MI03 | 2 | 19158691 | ATGCTACAGTTGTACATAAGTCTGCATGACCGTTT[A/G]AACTTTGAAGGTTTCACTAA |
| 45 | | 8 | TATGCATGGTAGCTA |
| MI03 | 2 | 19324631 | AGAGAGCTTGGCAAACCAACGAGCCTTGGCTCTCA[A/G]CCGTAGGCGTCACTTGAC |
| 46 | | 1 | CCTGTCCCTTTCCTGAC |
| MI03 | 2 | 19424701 | TTGAACATTTTTGGATTTCAAACGATTCAAGAAGG[C/T]TAGTTGGTGTGGCGCATACG |
| 47 | | 7 | TGGACCAATCAGAAT |
| MI03 | 2 | 20379484 | GATTCATGGGACGAAGTAATGACACTAGAGGACAC[A/G]AAAGGTATGGAGTTTTCGT |
| 48 | | 1 | ATCCTGCAGTGATCCA |
| MI03 | 2 | 20392715 | TGAGCCATATAGTGTTATGTTATCAGTCTTCTATG[C/T]TGGGATCTTGATAAGGCTGGT |
| 49 | | 4 | GAGTAACTTGTTGC |
| MI03 | 2 | 20512254 | CGGGTCTAAATGTCTAATGACTCGTGTTATTCAAC[A/G]ATTTAGGATTAAATTTTAGAG |
| 50 | | 3 | TGTAATCGACACTC |
| MI03 | 2 | 20553855 | CTCGATGGTGAAAGCATTACGAAGAGGCAGTACTA[C/T]TGTCAATGTATTACAGTTG |
| 51 | | 1 | ATTGCAATGTTTATTC |
| MI03 | 2 | 20558254 | TTTAAACGAACAAGAATGTTGCCAGGGTGCATATC[C/T]GCATGAATAAAATTGTCCTC |
| 52 | | 4 | CTACAGAATCGGGAG |
| MI03 | 2 | 20569623 | TGGAAGACGTTGGTGTGGACACGCCGGAGCCGACG[C/T]TGTTGTGGTTGTTACCGC |
| 53 | | 0 | TGCTCGTCACCGTGCTG |
| MI03 | 2 | 20569820 | AAATCTCTGATTATTTTCATGTCTTTTTGCTGCTT[G/T]GATATGTTCCGTCATCTTCTT |
| 54 | | 4 | CTTCTTTTTTGAAA |
| MI03 | 2 | 20688500 | CTCCCATCAGATATTGTGACGGTAGGGTTGAGGGT[A/G]GTGGACATCGAGGGGCAGA |
| 55 | | 2 | AGAGAAGATCAGTCGA |
| MI03 | 2 | 20992834 | GACCGCGCGCTTCGCGACTCTGGTTTCCTCCTCGT[A/G]TTCCACTGTCTTACGGGAC |
| 56 | | 1 | CCACATCACTCTAGAC |
| MI03 | 2 | 21050249 | TTCACTCTTCGAGCACCACTAATTAACGAAAATCG[A/G]TTCTTTAATATAGTCAAATC |
| 57 | | 2 | TACTTCCCAAATGCC |
| MI03 | 2 | 21150007 | TGATAGCTAGCCTAGGTGAAAATTTAGCTGGACGA[A/G]CCAGCGTTAGGATGATTCA |
| 58 | | 0 | TTAGAAGTTACTCGGT |
| MI03 | 2 | 21189704 | TCTCGGCCCATCTTACAACATGGAATGGGCCTGCT[A/G]CAATTCGGATAGTTGACGA |
| 59 | | 3 | GTCCAAAACAAACATG |
| MI03 | 2 | 21251396 | CACAACAGCTATACGCATAGAGACATCGCCACGTC[A/G]TTGGAATTTATGTAATCCGT |
| 60 | | 3 | CTGATGGAGGATCGT |
| MI03 | 2 | 21465758 | GCGGCGACATCTCCGGTTGGTGCCCGCTCCTTACC[C/T]CCTGTTCATGTTAACAGTAT |
| 61 | | 7 | TCGGTTCAGTTGGTT |
| MI03 | 2 | 21590669 | ACTTATTCTATTGGTGTTTCGTTTGACCTCAATTT[A/G]CAAAAGATGGCTCATAATTGG |
| 62 | | 9 | GTATGTTAGTATCT |
| MI03 | 2 | 21598457 | GCTCTCCATTGTAGAATCAGGTCCTGCAACAATCG[A/G]GGCAGCCACAAACACTGG |
| 63 | | 8 | ATCGTAGCCGTACAAAG |
| MI03 | 2 | 21669496 | TGTTAGTAGTGTGGTTGGAGGCGCTGGAGTCGACC[A/G]CCCAGTCGGTAACCACATC |
| 64 | | 9 | GGGTGGATTCAAAGCC |
| MI03 | 2 | 21776276 | AAGTGGATATTGCATGCAACAAATATATAACTCTA[C/T]CCAAGCCTCAGTAATTGCAC |
| 65 | | 1 | TTCTTATCACAGAAG |
| MI03 | 2 | 21913158 | GAATGGCAATGGCTGGAATTCCCGAGGCGACTCTC[C/T]TTCCGCTTCCAATACCTGT |
| 66 | | 6 | GTCAAGCGGTTTAAAG |
| MI03 | 2 | 21995284 | AGCAACTTGATTTAGTTTTAAGCAGTTCAATAAAA[G/T]AAGTTCACAACTAAAGACA |
| 67 | | 8 | ACACTAGGCATATAGT |
| MI03 | 2 | 22022380 | TTTTCCAATTGGTGCTAGTCCTAGCTTATCATATA[C/T]ATATCATGCGTTCCGAACTTG |
| 68 | | 8 | ATACCTTCTTATAT |
| MI03 | 2 | 22099392 | TGTTGCTTTGTTACTGCCTTTGTTTGAAACTACTC[G/T]GGATAAAGTCATGGAAATGT |
| 69 | | 1 | TTGTATCAACGTGCC |
| MI03 | 2 | 22203639 | TTCCTTCATGTGTCTGCCTGCCTCTCCCGGGTCCC[A/G]GCCCGATAAGGACGGCGGA |
| 70 | | 2 | GCCTTCATGCGTCTGA |
| MI03 | 2 | 22245084 | TCCTTCAGGTGATCCATCATTAAATGACAGAGAAT[C/T]CCTTGTCTGAGTTTGACATT |
| 71 | | 5 | GTTGTACTTTTTTGA |
| MI03 | 2 | 22369724 | GTCTCTGGTCTGTTGATCTGGAATCCAACGGTGGA[G/T]TGTGCTACCGGTTCGGTCT |
| 72 | | 4 | CTAATCTGAGTCGTGG |
| MI03 | 2 | 22480093 | ATATTTGGACATAGGAGGAAAATTTGGGTTTCTAC[C/T]AGAAGCACCAGGCCGGTCC |
| 73 | | 2 | AAGGGCACATCGGACT |
| MI03 | 2 | 22526485 | TTCTGTAATAGTTTAGCAATGCTCGTTTCTAAGGT[C/T]TAGGAAATCCATGTCAATTTA |
| 74 | | 1 | TTTTTAAGGTCTTA |
| MI03 | 2 | 22589832 | TAAAGGAGGGCCCATCCTTGCTTTCCTTTCAGCAG[C/T]GATGAGGAGGCGATGGCCA |
| 75 | | 8 | CATACATACCTCTTGT |
| MI03 | 2 | 22878858 | ACATATCACATAAAGAACAACTAGCTCTTTGATTG[C/T]GTTATATTGATAAACTTGGA |
| 76 | | 3 | AGGCCATGTGAGCAC |
| MI03 | 2 | 23034913 | AAATGCAGGCTTGCTTTATTGGAGATCCAGGACTT[A/G]TTGCTATTGGGGAAGGATG |
| 77 | | 8 | CAAGCTGCTTAGGAAA |
| MI03 | 2 | 23102767 | GGTTTCCGGTTGATCTCATGCATCCACAGATACCT[G/T]GCATCGCCAGACATGAGCGC |
| 78 | | 2 | AAGGGAGCCCGGGTT |
| MI03 | 2 | 23153733 | TCATCGGGGTTGATGCTGTTGCAAAGCTCCTTCCC[A/G]TCAAAGAGCTCCTGAAGAA |
| 79 | | 6 | GCTGCTGGATCTTTGG |
| MI03 | 2 | 23160422 | GCCCAGAACGACTCAGCATTGGGAGATTTGGCTGA[A/G]CACTCACAGTTCGGGAGC |
| 80 | | 4 | AGCAATTCCCTACCAGA |
| MI03 | 2 | 23313988 | ACCACAGTGATAGAAATCAACGCTGAAGTCTACGT[A/G]CGTCCAGGGACCGTCCAC |
| 81 | | 3 | ACATGAAATGAAAGGAA |
| MI03 | 2 | 23317005 | CTCACAAATCTCGACCAGAACAAAGAAACATGACC[C/T]GGATCAAGCTAGAGGATT |
| 82 | | 3 | ACTCGCCAACAAGTCAA |
| MI03 | 2 | 23426584 | ATTTTTTTAATCATCATCGTCGTGGAAGCTTATCT[A/G]TTCGCTTCGGATTCAAGTCGG |
| 83 | | 8 | GTGTTTGAACTGGT |
| MI03 | 2 | 23458550 | AATGCGTCATAACTTGGCCAATCATACTTCAGATA[A/G]TAATACTAGATGAAATTGAG |
| 84 | | 3 | AGCTGTATTACCTTA |
| MI03 | 2 | 23468381 | TGTTTTATTATGACCATATGATGTTTATCCTTTTA[A/G]TTTTTATCTTTTAGCATAATA |
| 85 | | 8 | GCATGTTAAGGTCC |
| MI03 | 2 | 23487931 | TCCTTACTATGTTGCCCCTGAGGTTCTGAAAAAAC[A/G]CTATGGCTGTGAGGTTGATG |
| 86 | | 3 | TGTGGAGTGCCGGTG |
| MI03 | 2 | 23520736 | GCAGCTCAATACCTGCTCACAGAAAAGTTTTGGTC[A/G]TCTGCAGGTATCATCGCCA |
| 87 | | 8 | GGCGCAGAGAGGCTTC |
| MI03 | 2 | 23707655 | CCCGGTCAGTGGTGTAAAAGCACTTCTGGTCGTCT[A/G]CCACCACGAGACAACAAC |
| 88 | | 2 | AACAGCAACAACAAAAA |
| MI03 | 2 | 23769656 | ATGGATTCAACAGTAATTATGATAGATGGTAACAA[A/C]AAACGTTGGCCACCCCTTAA |
| 89 | | 6 | ATTTTAAACATATGT |
| MI03 | 2 | 23779477 | CCCACAATATTTAAAGCCAACCACATGTCAAGATG[C/T]CCTAGCTATTTACTTTGTAG |
| 90 | | 2 | GCCAGGAGTCCAGGG |
| MI03 | 3 | 3921002 | AGATGGTAACTAGCGTGTGGACTTGGGCTCGTGCG[G/T]AAAGTGGGGCCCGGCAAA |
| 91 | | | GGCCCACTCCCCCGTCT |
| MI03 | 3 | 4154238 | ATTATTTTGATGCTATAGTTGCTTGTATTTTTTTC[A/G]ATAAGGTTGAAGAAATATTAT |
| 92 | | | CACAAAATGGCAAA |
| MI03 | 3 | 4194871 | CCATCCTTCGTGTACCCACAGCCACCCAATCGCAC[A/C]GCCTAAAGCAAAACACGTT |
| 93 | | | TAATGACGGCGTAGAT |
| MI03 | 3 | 6527555 | GGACTTGAAGGAGCTACGCTTGCGCTTCTGATTAG[C/T]CTCAGACGCAAGTGCTCTT |
| 94 | | | GTAGTCTTCACTTCTC |
| MI03 | 3 | 7684608 | TTAAAAAAAACATGGTTAAAATTCGAAGTAAAATG[C/T]AAAAAGTTTATAGATTGTAC |
| 95 | | | TGATAAGGCATCAAA |
| MI03 | 3 | 7775664 | TCTTCACGCAAAGTGCAAAAACTTTAACCCTAAGG[C/T]AACATGAACAGGTTGTGTG |
| 96 | | | TGTGCTGAAGAAAGAG |
| MI03 | 3 | 8606955 | CCCCTGTCGAAAACAGAGATTTCCACGTCAGCCTC[A/C]AGCAAAATGAAGTGGTTTT |
| 97 | | | CCTTCTATAATTACGT |
| MI03 | 3 | 9042692 | CACATTAACTTAGCCTATAGCGGCGACTGAGGTGC[A/G]GCTTGGTTAGGAAGCCAGT |
| 98 | | | TTTAGTTTTCTTTTGG |
| MI03 | 3 | 9909516 | GTCTATAGTTATTTTCCTTTTGTCATGAACTAATG[A/G]ATTGTGAGGCATTAGATAATC |
| 99 | | | TAGTTGTATTGGAG |
| MI04 | 3 | 9950901 | GGACATCCTTACAAGAGGACCCAACAAGGACTAAG[A/G]AAAGCACAAACTTTTTGA |
| 00 | | | CACCGCAACAAAAATCT |
| MI04 | 3 | 10184005 | TGACAATACCTATGAGTAAGTCAATACTGTAAAGC[A/G]ATAATTTTTCAAAAGCAGTA |
| 01 | | | ATATAGTATTTCGTG |
| MI04 | 3 | 10452903 | TAGAATATTGGAGTGAGAACAAAAAGGTTGTTTTG[G/T]TTTACAAGAACGTGTCATG |
| 02 | | | TTTCTACTCGAAAACC |
| MI04 | 3 | 12037442 | AGCTCTTGCTTGTAGAACTCACGCTTGTCAATTGA[C/G]GCGTGATAGATCTCGACCTG |
| 03 | | | GACAGAAGCTCAGTT |
| MI04 | 3 | 12321178 | AGAGTTATATCCTTGTATGGATTGTGGATCCTACC[C/T]ATTAGAGATCCTAATACTCCT |
| 04 | | | ATCTTGTTATAGTT |
| MI04 | 3 | 13470034 | GATGGACTGTAAATTTGTTCGTCACTCTTGCTGTT[A/C]TTTTCGTTCAGCTGCATGCA |
| 05 | | | CATGAGCTCATTGCA |
| MI04 | 3 | 13869033 | AGATCTTTGTCCGTGGTAACATTTAATACTCCATG[C/T]TGTACATTTAATACTAGATCA |
| 06 | | | CTAGGCCCACCTGT |
| MI04 | 3 | 13881769 | TGCCTGCTTGCTACGGTTTCAGAATGCTTTCTTTG[G/T]CATTTCGTAGCAGAAAACAG |
| 07 | | | TGCGCCACCTGAGCG |
| MI04 | 3 | 14418494 | AGAAGTAGCGAGCATCCAAGGAGTACGAGAAGGTG[A/G]ATCTGTGTGAGGAGATTC |
| 08 | | | AGAGCTGCTTCCATGGT |
| MI04 | 3 | 17438691 | ACTAGATTCTACAGGGGATGTCGAAAACTTGAACA[C/T]GTTCCGGCATCCATCACGC |
| 09 | | | AAACCAGTCCACCGAT |
| MI04 | 3 | 17805407 | ATAAGTAGTAAGAGCACATACGGTATTACACCATG[C/T]TGGCCTAAACTTGTGCCTAG |
| 10 | | | GTTCAATACGATGGT |
| MI04 | 3 | 18193032 | ATTTAATCAGAAGTTCGCAGCGTGTCATTTGAAAG[A/G]TAAACTGGCATGGACTGTG |
| 11 | | | CATGACCGAAGCTGTT |
| MI04 | 3 | 18810450 | GTCGACGTGCCATCGGTTCGGGAGAGCAGCATCTT[C/T]GTAGTCTATGCCCCCCGTG |
| 12 | | | TCCCTGCACGGGGTAG |
| MI04 | 3 | 19562535 | TCAATTGTACGCATGGAAATGAAATATGGTATCAT[A/G]CAGGTTTGGGGAGTGGTATT |
| 13 | | | TGTTAGCAACTCATA |
| MI04 | 3 | 22161510 | TTGCATCTCTGTATCGAGTATCAACAAAGAGCTAA[C/T]AGCCACCAGCCCATTACATC |
| 14 | | | TTTAATTTCTGAACG |
| MI04 | 3 | 23212790 | GAGGAGAGAGAAAGGGACGGCTGTTCGCAGAGCAC[A/G]CAGATGAACTGATAGAG |
| 15 | | | AACTTTCAGGTTTTCCAC |
| MI04 | 3 | 23848846 | ACAATCTACTTTGCATGGCATGTACTGTACGTTTT[A/G]GCCACCTTCTTGTTGCTCAG |
| 16 | | | AGTTGACTTGACCTA |
| MI04 | 3 | 28924037 | CTGCAGGAAATAAGCAAAGAACAAAAAAAATGTGT[C/G]ATCATCCCTACTGAAAAT |
| 17 | | | GTCAGATGGTCTCGCCT |
| MI04 | 3 | 30049902 | AGAGTTACGCACTCAGAAAAGAACATGCCATATGC[A/G]ACATTTCCACAAGCATGCA |
| 18 | | | TGACACTACCGCAAAA |
| MI04 | 3 | 31489331 | TCCATGGGATGGGATCAATACAAAATCACTGAACT[A/G]ATCGATTGCTTGAGACTGTA |
| 19 | | | CTGTCTGCATATTCC |
| MI04 | 3 | 32244563 | GAGACCAATGAAGCATCAGACAAATTTGGGTCCCT[A/G]ATCTCTATTTTCCAAATCAT |
| 20 | | | AATGACAGATTCCTT |
| MI04 | 3 | 35028048 | ATGCATATCATTCACAGTCACAGCGCGCTCTATAT[G/T]TCTCTGCTCTCTCTACCAGTG |
| 21 | | | CAGCGCATGGCAGA |
| MI04 | 3 | 35077416 | TGGTATGTCATTTGTATAAAAAATGAAGCTATTAC[C/G]CTGCATAAATATGCATTTCAC |
| 22 | | | TTATTATGGGCATG |
| MI04 | 3 | 35747767 | AATGACGAAGCTGTTTCTCTCTCCCTTCGTTTACC[A/G]AAGCTTTCAGTCGACGCTTG |
| 23 | | | CCTTTGTTCCTGAAA |
| MI04 | 3 | 35748583 | GTGCTGCTTCGTTCCAACGAAAACGCAACATGAAA[A/G]CAATGCAACTTTCACCAG |
| 24 | | | GATGTTTTATCATTTCC |
| MI04 | 3 | 36318636 | AGCAACGTGTGGTTAGCAACACAATTTTGCCCTTA[C/T]CACATCTCAACAAAACCCC |
| 25 | | | AAATACACATAGTTAC |
| MI04 | 3 | 36947375 | CCCCTTACATATGCTTCAAATTGGCAAATGGGAAG[A/G]TCACCCGCAGTTCCTGGATC |
| 26 | | | ATATATAGCAGGCTC |
| MI04 | 3 | 38438567 | CAGATTAGCGCATGCCATAACTGCTGTGACAGTAA[C/T]AGTCTCGTGACACGTAACT |
| 27 | | | AGCTAGTACTATTACC |
| MI04 | 3 | 40929565 | TGAGCATGATGTCGGCGACGTAGAGGAGGTAGACG[A/G]TGTTGTTGCTGTGCCGAA |
| 28 | | | GGATGAGCAGGGACATG |
| MI04 | 3 | 42552560 | TAATTCCGCCTAATCAAGCCACATGTCCCAGATAA[A/G]GTACTGTCGGCGTTTCGAGA |
| 29 | | | CCGGGGGTCCCTGGG |
| MI04 | 3 | 42890120 | GAGACACGGAATCATGAAGCAAATATTAGGCGATA[A/G]ATATAAAAGCATATTACACC |
| 30 | | | GTATCGGAGGAACGA |
| MI04 | 3 | 44264231 | CTAATCTACGCCTTGTTCACCCTTTGCTCCTCCTA[C/T]CAAAACTCCCAACTCCAACC |
| 31 | | | AATTAGCAAAAACAA |
| MI04 | 3 | 47071994 | TGGAGCCCATCAGCTTAGATATGAATCCATGCAAC[A/C]GTGCATCTTATACAGGTTAA |
| 32 | | | TAATGGAACTCTAGG |
| MI04 | 3 | 49500370 | TTTACAGTTCCATTAACTTTTTTATACTTGATTTT[C/T]GCAAATACAACATCATTATAG |
| 33 | | | AGATAACAATCATT |
| MI04 | 3 | 50563756 | GCAAATCATCTCCAGCGGCACTCTCCTCGCCTCCC[C/T]AGATCTCGGTTCTCCATCAA |
| 34 | | | GATGTGATGACTTGC |
| MI04 | 3 | 51765091 | ACTAAATGTACAAAACTACAAACGTCATTGATACA[A/G]ACAATAAACACTACTATTCT |
| 35 | | | TGGGATTCATGCGCC |
| MI04 | 3 | 55108926 | CACTTAAGCAAGGATTCACCTCTCTGACTTGAATC[C/T]TACAAGCTTCTCATACAACT |
| 36 | | | TGAACTATTAGAGGA |
| MI04 | 3 | 55959796 | AGGCTTAATCCGTGGCTTAAATAGCGGATGAGAGG[A/G]TGAGCTGAGCAGATGTCGT |
| 37 | | | GGGAGATGAAATGCAA |
| MI04 | 3 | 57545518 | CTATAGCCTCATAAACTTGTCCACATGCTTTAACT[A/G]GGATAGCTGTAGGGATGGAT |
| 38 | | | AATTGGATCTCTTCA |
| MI04 | 3 | 59555371 | CAAGCTCACACAGACTGAATCACATACTCAATACA[A/G]CTTACACACAGTGGAGGTA |
| 39 | | | GGGTATTACGCTCCGG |
| MI04 | 3 | 60878936 | GATGGAATCATTTGTTACAAACATCGTATTGTGAT[C/T]GCAGCTTGGTCCGAATTTCCT |
| 40 | | | CAGAAAATTTTTCG |
| MI04 | 3 | 64815690 | TCCTAGGTGTTTTCGTAAGATTCTTGCGCTGGAGT[A/G]GTGCTAATCATCATGCTCAG |
| 41 | | | CTCTCCAGGTCCAGC |
| MI04 | 3 | 64821150 | CTGACCCACGCTCAGAATCCATCCATTTCTATCCC[A/C]CACACACAAATACACATCCC |
| 42 | | | CCTTCCCTTCCCCGC |
| MI04 | 3 | 66658033 | TTCTCCAAATCGCCCAGTTTTTGTGTCTTCACTCC[A/C]TGTGTATGTATTTATTCACAT |
| 43 | | | TTCATATACTTATG |
| MI04 | 3 | 67259018 | TTCTCTCCTCGTTTACTGTGTCATCGCAACCCACT[C/T]CCACCGACTATGTTGCTAAC |
| 44 | | | TCTTCCTCATACCCC |
| MI04 | 3 | 75808466 | GCCCATGCGCGTGACAATGGCGGAGGAGCTGGCGC[C/T]GGTGGGGTCCGCCTGTAA |
| 45 | | | TTCATGGAAAAGGCTGC |
| MI04 | 3 | 77851903 | GTTCCCTTGACTCCGAGACTATTGGATTCAGGACG[A/G]ACGACATCATCGAGGCGGC |
| 46 | | | AGCTTTGAAGACCCTA |
| MI04 | 3 | 82739532 | ATTGGAAACATTAACTGAATTTTAGTGTTGATAGC[A/C]GTTTTGGAGCTGATTTGTGG |
| 47 | | | CTGTTCTATAATGTT |
| MI04 | 3 | 85891476 | TTAAGGCTTTCTACACTTTGAATTGCTGAGAGGCA[C/T]GAGAATGAACATACACTCC |
| 48 | | | GATCCGTGTTATAAGA |
| MI04 | 3 | 86929806 | GTCTAGATGACTCAATTGAATAAGGTTCCATTCCA[C/T]CGCCTAGACCGTGGTGGTGA |
| 49 | | | CGAAGAATGACGTCT |
| MI04 | 3 | 86945299 | AAAAGTGGACCCACGCTCAATGTGTGATGACCATC[A/G]CAAACGGGGTTAGTCCCTA |
| 50 | | | AACTGCGCAAACATCA |
| MI04 | 3 | 89380527 | CCCTTCCTGACGTCTCTAGTGATCCTCTTTCTAAA[G/T]CCCCTCAGCCACCCAAAAAG |
| 51 | | | AAAGCCTCCTCCCAC |
| MI04 | 3 | 91465356 | ACAGTTTATGAAGATGAAGTAAGAACTGATCAATT[G/T]GAAAAAAAAGATCGAAGC |
| 52 | | | AATTCAAAAACAACTAA |
| MI04 | 3 | 92064494 | TACCACAGGTTGTTTGGCCGAAGTTGGAGCTGTGA[C/T]GACTCATAGAGCAAAGAA |
| 53 | | | GATCAGTTTTTCTCAGG |
| MI04 | 3 | 93203422 | TGCCTATGTCCTAAACTACTTATATTTGCAATCAG[A/G]GGGAGTATTTAAACATGTGTT |
| 54 | | | TGGTTTCGGAACGA |
| MI04 | 3 | 94583387 | TTTTATCATTTTAAATCCCAAAATTTGATACCTGA[C/T]AGAGGTACATCTTGTCCGATG |
| 55 | | | TTGAACCCATCACG |
| MI04 | 3 | 10089926 | ATCAAAACCAGCAGCAAATCTTCGGCCACGGCCGC[C/T]ACCACGGCCACCACAATG |
| 56 | | 0 | GTGATCATCATCCTCAT |
| MI04 | 3 | 10360649 | GGACTCCGCTCCTACACCATGGTCACGTTAGAAAC[A/G]CAGACTTTGTAATAAAAAA |
| 57 | | 4 | AACTCACTAGCAGGAC |
| MI04 | 3 | 10684041 | CTCGATCGACCAAGTATGTTGGTCTAAGGCCCGAC[A/G]CAGCAAGGTGTCGAGAGC |
| 58 | | 5 | ATCATGGAAATGACAAC |
| MI04 | 3 | 10733621 | GCTTACTTTACAGTTGATAACTAGATGGACCTTCT[G/T]CCGCCCAATGTGCTAATTTG |
| 59 | | 0 | ATATGTTCTTGTTTC |
| MI04 | 3 | 11224679 | ATGATACAAACCTGAAAAGTTTCGCATAAATACCC[C/T]CCTCGTAAGATAAAACTTTA |
| 60 | | 0 | TGAGCTATTGTTGGG |
| MI04 | 3 | 11349117 | CTTTGTTGTTGACAGAGAGGTAGCATCAACAGCAC[C/T]AACTCTAACTCTCTGAGAA |
| 61 | | 0 | GAGAAGTCCCAAGTTC |
| MI04 | 3 | 11432404 | TGGGTTTTTGATGAGCTCAACAACCCTGAAGGCAC[C/T]GGATACTACTGAACTAAGA |
| 62 | | 7 | TATTCTTGCATCAAAT |
| MI04 | 3 | 11437492 | CACTCAAACTAGAAGTGAGTTCAACTTAGAAGCCA[C/G]ACTTGCTTCAAGACATGA |
| 63 | | 2 | AGCAAGTCAATGGCTGA |
| MI04 | 3 | 11489199 | ACAAAAATTCTCGAAGAGTGATACAAATTGAACTA[A/G]ATTGGGAAAATAAATAGGA |
| 64 | | 4 | ACCGAGCACATCGTTT |
| MI04 | 3 | 11620904 | AAAGGCAGGGCGCAACTGGAGGAGCAGATAAGAAG[C/T]AGCGCGCGCAGGGAGCT |
| 65 | | 8 | TCGAAGGCGGTGACTTGG |
| MI04 | 3 | 11638048 | TCGTCCACGTGCTTTGCGAATTTGGGACGGAAGTG[C/T]ACACGATGGTGAACAGATG |
| 66 | | 1 | AGTCGCCAGAACTCAT |
| MI04 | 3 | 11753794 | TTATGACTAACACCTAAGTATTTAGAAAGCAATAA[A/C]TTTATAACTGTAACTCCGAA |
| 67 | | 0 | TTTAGTGGTTCTCGA |
| MI04 | 3 | 11760405 | ACTTTTTTCAGTGACAAATGTTTTAAAGTCTTAAA[C/T]ATCGGATGTAGCTAAATGTA |
| 68 | | 5 | CACTATTAGCAACCA |
| MI04 | 3 | 11778843 | GAGAGAGAGATAGATAGATGTATAACCCCACACGC[A/C]CACACAGTAGTTCTCTGTT |
| 69 | | 9 | CTACCGTGCCGTCACC |
| MI04 | 3 | 11883623 | AATGAAGACTTTATTGATGACGGATCCTCCAATCA[C/T]AGAGTACAATGTTGCTCCGA |
| 70 | | 3 | TAGCTTTTGAGGCCT |
| MI04 | 3 | 12075991 | GTCATTTACACTTAGCAGCTTGGACAACACCTTCT[C/T]CTTGCGTCGCCTTCCTATGT |
| 71 | | 4 | CAACGTAACGAAGCT |
| MI04 | 3 | 12264596 | GGACGGGTGGGGGAGGTTATGCGAAGGCTTATGTC[A/G]CTTAAGAGTGAAATTCTTG |
| 72 | | 2 | ACCAGGATATTATGAT |
| MI04 | 3 | 12328348 | AGGGAGTGAATATACAGATCCGACAAATACTCACC[A/G]TCTTAGCATCAAGCGGTAT |
| 73 | | 2 | CACACGGAAGCCTGAG |
| MI04 | 3 | 12811979 | AGACAGAAAGATGATTGCATAGGGAGATACCAAGT[A/C]TTCCACTCTACTTATCTGTA |
| 74 | | 2 | GAGACATGCCATGTT |
| MI04 | 3 | 12883166 | TACCCCCTATGTAATGAGCTTGACCCAACTACCCT[G/T]TCTATATTAATACACTCCCAA |
| 75 | | 1 | CCCTAGTTAGGTTT |
| MI04 | 3 | 13116138 | TTCCCTCGTCGCCACCTCGCCGAGGCTGCATGTCT[A/G]CTGGTCAGGTCCTAGCTAG |
| 76 | | 0 | GGACAAGAAGGGACGG |
| MI04 | 3 | 13132677 | AAAATAGAATAGAATTAAAACAAGGGGTGCTGAGA[G/T]TTATCACGCATCATGCCAT |
| 77 | | 7 | GATCGCCAGTGTGTTC |
| MI04 | 3 | 13152975 | CCAAACTAATTAATGACAGCTAAACAATGATGCTA[C/G]TACTATGGCTCCTGCCATGG |
| 78 | | 7 | CCTATACTACAAAAA |
| MI04 | 3 | 13242327 | ACCACATAGAAACACTGATGGCTACTATATGATGA[C/T]GAACACACCGATGGTCATCT |
| 79 | | 0 | TTCAGAGTATCGCAC |
| MI04 | 3 | 13553332 | AATTATCATCGATGGAACGCAAACAAGGAGAGCAC[A/G]AGAAGACATCTGAGTTCC |
| 80 | | 6 | TGCAATCTAAATTGGAT |
| MI04 | 3 | 13572001 | AGATCGAGGCCGCCAATTGCACGCCATCGGGTATG[A/C]GTTGGGAATGAATGTCGCC |
| 81 | | 0 | GCTGTTTTTCGGCCAG |
| MI04 | 3 | 13572576 | CATAATTATTTCTTGAATTTAGTTTTTTTGTGGTA[A/T]TTTTCCTTCACCTCTTCCCCT |
| 82 | | 0 | CACGATATCTTTTG |
| MI04 | 3 | 13615343 | AAGTTGGAAGAATACGAAAGGAATCAAGCACTCAG[A/G]CAACCGAACTGGAAGCA |
| 83 | | 9 | AGGAAAGAGGATCGACGA |
| MI04 | 3 | 13658447 | GGCTGCCTAATCCCAAAACAATGGGCTAGAATCTC[C/T]TCTTTCATCGACTAAAAATA |
| 84 | | 8 | CTAAAGTGTCATTAT |
| MI04 | 3 | 13660949 | AGCGTTACGGCATCGTCTACGTGGACCGTAGCAAC[A/G]GCTGCAAGCGCCGCATGA |
| 85 | | 7 | AGCGCTCGGCCAAATGG |
| MI04 | 3 | 13737030 | GATACTAATTGGTCGAAGTTGGTATCAAACGGTTC[C/T]CCAGAGCGCGAGTCAGAAC |
| 86 | | 7 | AAGCCAACCGATCAAC |
| MI04 | 3 | 13976125 | TTAATAGCTAAAAGTGGTACAGAGTACAGACATAC[A/G]GCTTTCCACCCAGAATGAT |
| 87 | | 6 | TGTTTTAACAGCTTTT |
| MI04 | 3 | 14374478 | CCGAAGCCAAAGAAAAAATGGCCACAGTGCCGAAG[A/C]TAGGGGAAACAGTAGGG |
| 88 | | 3 | CTGCCAAAAATCTTGAGC |
| MI04 | 3 | 14463794 | GTGCACTCGTTTTCTTTCTTGCGGCTCAAGACACC[C/T]TTGATGTCCCTTTGTTTCGG |
| 89 | | 6 | TTTAAATCTAAATTT |
| MI04 | 3 | 14585673 | TAGGGATGGACGACGGCAAACCTCATGATCTGCAG[C/T]GGCATTGCTATTATTCCTTT |
| 90 | | 9 | TCAGGGAGCGAGACT |
| MI04 | 3 | 14606346 | GTCCGGTGATATTTATAGCAGGCAACCAGCAAACT[C/T]AACAACTTCGGGATTTTCG |
| 91 | | 7 | GGAGGCAAGAGCACCA |
| MI04 | 3 | 14704949 | AGAAGGGGGAAGACAAGTATCCAAATACTAAAAGC[A/G]TAATAAGTTCAGAGCTTA |
| 92 | | 4 | GAAATCAATCACAAGAT |
| MI04 | 3 | 14778180 | TTACCATTTTACCCGAAGTTGCCATTTCTAGCCTC[C/T]TTTTCTCTTTTGGACTTAACA |
| 93 | | 5 | CCTTCAAATTGCAT |
| MI04 | 3 | 14888455 | CAAAACGAAAATGTAATCCCTAGTACAAGGACGCC[A/G]CAAACTCACACCACAGAC |
| 94 | | 7 | ACTCGTGCGTCCAATAC |
| MI04 | 3 | 15009598 | CTCGATATCGCCGCCACCAGAGCTCCCGTCCATAT[A/G]CTATTACCATTTTGGGCCTG |
| 95 | | 1 | CTTGATTTCTATCCT |
| MI04 | 3 | 15329786 | AGAGCTCTACTATAAGATCCGACTTGCGGCCCCAA[A/C]ACGCAACGTCTCGGCAAGG |
| 96 | | 9 | TTCCTCGCAGCCAAAG |
| MI04 | 3 | 15380779 | CTCGAGTGCCAAAACAACAGCGGAGAAAAAAGAAT[C/T]AGAACTTGAAATGCTTCC |
| 97 | | 5 | ATAATCTACCCTTGGCC |
| MI04 | 3 | 15460115 | TTGGGCAAACAAATGATATTCCATTTGGCCAACAT[A/G]TATTTCTTCTTGTGCTCATCC |
| 98 | | 0 | CCTTGTCAGTAAAA |
| MI04 | 3 | 15474818 | CAGCCTGCGTTCTTGTCGTATCGTACGTCCCATCG[G/T]GGGTTGGTAGAAGGCGTGC |
| 99 | | 3 | GGGAACAGTGGTCACA |
| MI05 | 3 | 15590075 | ACTGGTTGGGAAGGGAATGTTTCTGCTGAAGAAGG[C/T]GCTGACACAGGTATATGGC |
| 00 | | 0 | TCGCCTTATTGCCTCA |
| MI05 | 3 | 15600237 | ATAGCGAGCCAGCTGCATCCCAAGTGTAAAAAAAA[A/G]AGAGAGGAAACAGCTGC |
| 01 | | 1 | GAGGCTTACCCTTTTTTT |
| MI05 | 3 | 15697889 | GCTGTTCATAGTGGTAGCCCAGAGATCTGCAGTTG[A/G]GCTGGTCAATTGACCCGGC |
| 02 | | 0 | TAAACAGCGCAACGTT |
| MI05 | 3 | 15849328 | CCTACTGAACCTTTCCCTAGTAAAATGTAGTAGCA[A/G]ACAACTCTCCTAGGTAAAC |
| 03 | | 5 | AAATATACTGAAACAA |
| MI05 | 3 | 16039758 | CAGCTCAGCTCAGATAGCCTGACTGAACTCCTACA[A/G]TCAACCAATCAGATGGATA |
| 04 | | 2 | ATATCTTAAGCCGGAG |
| MI05 | 3 | 16613670 | CTGGATGTAGCACATCAGATTTGTAGACCAAAAAT[A/C]TCGGCCTGTTAGCTGTTACC |
| 05 | | 8 | AAGCCTACATGGTGT |
| MI05 | 3 | 16851981 | AACATTGTGCGATTTTCTTCATTTTCTAGCGAAGC[C/T]GACCTTTGCAGCCACGGCGC |
| 06 | | 2 | CCGTGGCACACCGGA |
| MI05 | 3 | 16853365 | ATGCCGCACACACAGGCTTCTCGTACGATCATTTT[G/T]AGGTGTAAATATGAGAACGC |
| 07 | | 6 | CAATCTCATACATCA |
| MI05 | 3 | 16856742 | CACCTAGTTTAAACTGGTTTTTAGACGCTGGAAGG[A/T]CTCCAAACATGTTATTTTTT |
| 08 | | 4 | TTTATCCGGTTTCTA |
| MI05 | 3 | 16864821 | CGAGCGGTGTCATACCTCCGCTCACTCTAGATCAC[C/T]GGAGGGGGTGAAGAAGGTT |
| 09 | | 6 | CTCATTGGAAAGGGAA |
| MI05 | 3 | 16890559 | AACTGGCCCCTGTATGGATTGTACTGCCTTCAATT[A/G]TGGTTTGATACTGAATGTGC |
| 10 | | 6 | TGAAACGACCGGTTT |
| MI05 | 3 | 16942178 | TCTATCGTCGGCGTTTGTTTACTGCGCCAGGACGG[C/T]ACGCCGTAAAGGTTTGAAG |
| 11 | | 0 | TGCAGATCGATCTCAC |
| MI05 | 3 | 17554010 | GTGCCTGCCACGATCATTAGGCCCAGCCACGTGCA[A/G]GATGTTCCGGTCGTATGAT |
| 12 | | 9 | GAGGCTTTAACCGTAG |
| MI05 | 3 | 17572915 | CAGATCAACATTGGTAGTTATGCAACCCCTCCAAC[C/T]GCAACTGTTTTTGCATCAGC |
| 13 | | 2 | CTCTCCTACCTTGTC |
| MI05 | 3 | 17636418 | AAGCAAGCACAAGAAAACTCAAGCAAATTCGTCCT[A/G]CTTAGAGAACTGGCTGTC |
| 14 | | 5 | AGGAGTTGTGGAGGTAG |
| MI05 | 3 | 17664584 | CTTTAGGTAGCTGTTCTACGAGTATTGAGTGGAGA[G/T]ATCAGAGATGGGATGGCGA |
| 15 | | 0 | AGTGTGCATACTCGTG |
| MI05 | 3 | 17664600 | TACCCTGAAGTGACTGTGGTTAAGTTTTATAATTA[A/T]TTACGTGCCGTAATCCCATAT |
| 16 | | 7 | GCAGTTTACCTTCC |
| MI05 | 3 | 17684414 | GTCTCAAAATTTATATTATCTTTCTAGCATCTTAG[C/T]TAACTGAAAATCCAGAAGCCA |
| 17 | | 4 | CCGATAGCTACAGT |
| MI05 | 3 | 17903767 | AAAATCGATTCAATGAAAGTTTTAGATAATTCGAA[C/T]CCCCAGAAGCCGGTAGTGC |
| 18 | | 1 | GCATGGCAAATTTGTG |
| MI05 | 3 | 17978295 | GCGACGTAGCCCGAGCCCTGAACTCTGAACTACTG[C/T]TTAGCTGAATCCTGTAATTT |
| 19 | | 6 | CAGTTTTTATTTCTT |
| MI05 | 3 | 18072083 | GCATGTGTGTGTGCAAGTGCAACAATCAGCATGCA[C/T]ACGCCTTGCGCCCTTGCCT |
| 20 | | 6 | CCTCCATTAATTAATG |
| MI05 | 3 | 18161860 | TCGGTCGGTCGATTTGCAGTGCTGCCTTGACCCTT[C/T]CAGGCAGCTACCATACCGTG |
| 21 | | 5 | AAGCCGTGAAGGAGC |
| MI05 | 3 | 18184451 | CAGGCCGTTGTCTCAGCGTCAGCGATTCCTCTCCA[C/T]TCGTCAACAACCCTTTTTCC |
| 22 | | 3 | TCTGGAAGCAGGAGG |
| MI05 | 3 | 18218384 | ATGGCACCAACTTTTAGGAGTGGCTAACTATGCTT[C/G]GTGTATGTTCTAACATAGAG |
| 23 | | 9 | GACGAGATATACAAT |
| MI05 | 3 | 18367179 | TCTGACGAAAACTTCCTCAAGGTTTGCCCAGTTCT[A/G]GATACACTCGGGCCTGAGG |
| 24 | | 2 | TTTTCGAGCAAGGCTC |
| MI05 | 3 | 18532812 | GCTGCGGCGCTGAAAAGATCTTCTTGGGTAGGTCC[A/G]CCGAGCCGATCGTCGGCA |
| 25 | | 0 | ACGTCACCTGGAACGCT |
| MI05 | 3 | 18722648 | TGTATATCCTTCTTCCTAAGATTATTTCAGAATCT[G/T]CTCAAGCCGAACATAGGTTTG |
| 26 | | 8 | ACAGACCGCACAGC |
| MI05 | 3 | 18745002 | CTATGATTAAGTACTAACTATTAAACTTAACAATA[C/T]AACTATAAATCTTGAACCCAT |
| 27 | | 7 | TAGGATATAGTGCC |
| MI05 | 3 | 18804262 | ACAATGGATGGAGCTACATGGTCGAAAAGCTTGGT[C/T]CTCAGGGAAAGCGTGGTG |
| 28 | | 2 | GAGGAGGGGGCTGGAAG |
| MI05 | 3 | 19017295 | GACGTGATTTGCTAAAATCTAGACTTAATTCTAAC[A/G]ATTTTACATTCCTGGTGGAC |
| 29 | | 5 | TGCGGGTTGATTCCC |
| MI05 | 3 | 19090001 | TCACATCTATGTGTATATCCTCTCCCTACGACTAC[A/G]AGCCTTAAGTTGCTGTCACTA |
| 30 | | 7 | AGGCCAGTCCTCTG |
| MI05 | 3 | 19183059 | CACAACAGAAAAAAAAACGGCCACCGGCAGCCCCT[C/T]AGTTAAATAAGATTTTTA |
| 31 | | 8 | GTTGACCAGGGCAAGCG |
| MI05 | 3 | 19208551 | TGTTATCCATGAGCTACCTAGGTTCGAGGCGCTTC[G/T]TGATGTGTGTGTCAGCTGAT |
| 32 | | 6 | CGGGTGGAAGGACAA |
| MI05 | 3 | 19410370 | AATTCTCAAATTTTGTCTTTTGCAAAAGAGGACTA[A/C]TGGACATTGAACTGATCGTA |
| 33 | | 8 | GGCTTGGAGGAGGAA |
| MI05 | 3 | 19429403 | CCGCGACCCGCGTTGCTGAGCCGCTCTTTTTTTTC[C/T]CCAGTCACGCACGCTGTTGT |
| 34 | | 7 | CGTTTCTTTTTAAAG |
| MI05 | 3 | 19663507 | TAGTATTCTATGTTCACGATGTTTCATCGAACTAG[C/T]ATTAAGTTGTGTTTATACTAG |
| 35 | | 5 | CATATGAGATTGTC |
| MI05 | 3 | 19934706 | GTTGACTGACGCTGTCCAGACTTATATCAGTTCCC[C/T]TACGGCTTTTCCTATATATAC |
| 36 | | 9 | TCTGCCCTATAAAT |
| MI05 | 3 | 20010056 | ACGCCCAGAAGGAACAGGGCTGAGAGACGCATGCA[A/G]CCAAGCACGCTGGAGCC |
| 37 | | 8 | CACGTACAACACAACAAT |
| MI05 | 3 | 20250164 | CATAGTGTTGTTCTTCATTTTTTTGTGTTGTGTGC[A/G]CCTATGTTTCTCTCTTTGTGT |
| 38 | | 0 | TGAGTTGCTGGTTT |
| MI05 | 3 | 20350701 | TGTTCCTGTCCATCCTTCAGGTCTGTCATCCTTGC[A/G]CTACAATGCTTCTGTCAATG |
| 39 | | 7 | GGTGTTCTCTTTTCG |
| MI05 | 3 | 20465999 | AAACTATTACTGTATGCCAGCTAAAACTTTCGTTG[C/T]AGATGAAATTAAGAGGAAG |
| 40 | | 9 | CAACTCCTGAGAAATT |
| MI05 | 3 | 20518219 | CTTCCTAACGTGATAGCCTAATACAAATTGGGACA[A/G]CAAAGGGCAGCGCAATTTG |
| 41 | | 0 | TCACCGTAGCTAAAGC |
| MI05 | 3 | 20676243 | TTTTTTTCCCTCTCCTTCAGTCGCATGCTCTTCCT[A/C]ATATTTATGTTCCTTTCTTTC |
| 42 | | 7 | CCTCCCTGCGTGCA |
| MI05 | 3 | 20905213 | ACTTCACTTCAGAGATGTTGCCTCTATGGATGCTG[A/C]CATCGACGCTCGACGCTGAT |
| 43 | | 2 | ACAACACCACTGACA |
| MI05 | 3 | 20954137 | AAGGGATCGTCCCTTCAATTGTTGCACAGCCAGCA[A/G]TACTTTGTCTCTATCATCAG |
| 44 | | 5 | CATGGACAACCTCCA |
| MI05 | 3 | 20991056 | GAAAAGTGTCCTGTTGTATAAACAAAGATGGTAGC[C/T]CTGATCCCTGGTTAGTGGT |
| 45 | | 4 | CACCCAGATAGTAAGG |
| MI05 | 3 | 21084436 | AATCAAAAGAAAGAAACTACAATGGTGAGCCCATT[A/C]AGCGGCTAGCAAACCAAT |
| 46 | | 9 | ACATACCACAGATACCA |
| MI05 | 3 | 21115654 | ATAAATAATAATAGTTTCACATATATTTCTTTCAC[C/T]GAAATCACATACATAAATTTT |
| 47 | | 5 | AGTGATCGACGTTG |
| MI05 | 3 | 21227423 | AATTGGCGCCTCCGAAAAGGTGTCAACACATGTTT[A/G]TTATCTCTGTGTAAACTGA |
| 48 | | 9 | GTTCTGTTGTATCTGG |
| MI05 | 3 | 21330433 | CTTCCGGACTTCGATTTAAATCTAACGCTAACTCT[A/G]GCCTTAGTGCGCATTTAAGT |
| 49 | | 2 | TTATAAATTTCAGGT |
| MI05 | 3 | 21493407 | TGCTTGTAATACAACCTCTCTGCAACTACGGTCCG[A/C]CGTCGCACAGAATTGGACG |
| 50 | | 0 | GGTCCCTATTCATTGG |
| MI05 | 3 | 21494293 | GCGGGGGTCCGAAGGCAATTCATGAATGGAGTGAC[A/G]ACGATAGGAAGCTAGTAC |
| 51 | | 7 | AAACTTTTGATCCATCA |
| MI05 | 3 | 21500922 | TTCTTTCATGATCTTGGCATTCGACTTTATTCCAT[A/C]TTGTGTCTTGTACTCTTGCAT |
| 52 | | 5 | GTTGTTGTAGATCT |
| MI05 | 3 | 21542032 | TTCTTTCCGTATCTAATCCGTCGGCAAACTGAATC[A/C]TGAATCCCCTTTCAGTATTAA |
| 53 | | 3 | CCTTTTCCGAAGCT |
| MI05 | 3 | 21585827 | AAAAGAAATCAACAACAAGCTCACCAATAGTTTGC[C/T]TGGCTTATTTTCCTTTCTTT |
| 54 | | 7 | GTATGTTGCTCTAGA |
| MI05 | 3 | 21634954 | GCAGAGTCTCTGGTGGAGGAGATCGCGTCACCAAG[C/T]GGGAGCTCAAGGTACGAA |
| 55 | | 1 | CTCTGCTCCCGTTTCAG |
| MI05 | 3 | 21687531 | GGTGGACGAGCTAGAGACCGTGACGACCTTGCGGA[A/C]ACGTACAAACCTATTAATT |
| 56 | | 7 | GGCCATGCCGACGAGG |
| MI05 | 3 | 21764662 | GCTGAATTGGCACTGTCATTAAGATTCAATCCCTT[C/T]TGTTTTCATGTTTTTCCCCTC |
| 57 | | 0 | ATCCCTGCCGTTCT |
| MI05 | 3 | 21809087 | ATGGTTTGGATGGCGGCGTGCACGGTATGGGAGGC[A/G]GTGGCGATTGGAGAGTAG |
| 58 | | 3 | ACTGAGGCTGAGAAGAG |
| MI05 | 3 | 22060440 | GTCCACGGTCCACAGAGACAAATCTGTGGCTGTGA[A/G]CCATGCACCTCCGCACGC |
| 59 | | 6 | GATCTTATCCTAACCGA |
| MI05 | 3 | 22172368 | ATATCCTTGAACAGTGTGAACGTTGAAGCCACATC[A/C]CCTTTCTTAGCATAAACAG |
| 60 | | 9 | ACATCAATGCGTTCCA |
| MI05 | 3 | 22566796 | GCAGTAGAAGCTGATATGGATTAAAGTCAAGCGAA[C/T]AGGCTCTAATGCATAGAGA |
| 61 | | 7 | CAAAACGACGGTTGAA |
| MI05 | 3 | 22656637 | AAAAAAAAACTATTGACCAGGTGTTGAAGTTGTAT[G/T]GTGAATTATGATATTGTCCT |
| 62 | | 2 | TGTTCTGTTTCTAAT |
| MI05 | 3 | 22659761 | GCTTTCATAAGTCCACTTCTAACGAGAATCACTCT[C/T]GTACAGCCAAACGGTAAAA |
| 63 | | 2 | AAGTGACATGCTTCAT |
| MI05 | 3 | 22665557 | AGAGTCATGGAACACCTTGCGCTTTCCTTTGAGTG[C/T]GGTGCTTGGGTCTGGTGTA |
| 64 | | 6 | CAGTTTTCACCCACAC |
| MI05 | 3 | 22688511 | ACCATAGGGTCAAGTAGTATGAACATGAATAAGTT[C/T]CGTAATGATCTAGCGAAGGC |
| 65 | | 8 | ACTTTGCGAGTCGAG |
| MI05 | 3 | 22771166 | TCCTCGCAGAGGGGGCCCTCGATGACGATCTGCTG[A/G]GACCCTGTGCTGCCCTGCA |
| 66 | | 4 | ATATATATAATAATCG |
| MI05 | 3 | 22804273 | AAAGAGGTCTCTACGAGAATATCTTAAACCTCATG[C/T]TGACGTAAGTTCCTGACCAT |
| 67 | | 3 | TTTTTTGCCTGTTTG |
| MI05 | 3 | 23194752 | TTGGCTAGAGAGACCATAGACACAATGTTTTCTCG[A/G]TTCCAGTCGATTATCAACA |
| 68 | | 3 | ATATGCGTGCCAATAA |
| MI05 | 4 | 596469 | CTATAATATATATCGCTGTGGATCGGAGTACATGG[G/T]CACAGTGGTGACGGATGGAT |
| 69 | | | GTATGTATGCATGTG |
| MI05 | 4 | 2414811 | GTATACTTTTAGTGTTTTTGTTTAGCTAGAGATGC[G/T]AGTGTGTTTGCTTGTTGTTGT |
| 70 | | | GCACAGTTTGTGGT |
| MI05 | 4 | 4483125 | CTTGCTGGCCACTCGAGTTATAGAAAGTTGCATTT[A/G]TGTGCATCCCACGCCAGCC |
| 71 | | | AAACACTGCCGACGAG |
| MI05 | 4 | 5387279 | CTCACCTTTTTGCCAGCGATGGGCACGATCCTGAG[A/G]GTGCCGGAGGCCTTGTCAA |
| 72 | | | GGACGCCAAGCGCGTA |
| MI05 | 4 | 6396382 | GCAAAACTGCATCGGAAAAAAAGATGGGTGGCTGT[C/T]TCTTCAGCTTGAATAATAC |
| 73 | | | ATTTGTCACAGGTGTA |
| MI05 | 4 | 6631372 | AACAGCCAGTAAAAGTTGCGGCCACCAAAGAAAAC[C/T]GTGTGCCCCTTTTCTCCTT |
| 74 | | | AAACTAAGGAAAAAAG |
| MI05 | 4 | 6647617 | GATGGTAGGCTTCAGTTATCATAGGATATGTTATA[A/G]CATCAATATGATGAGGCTAAC |
| 75 | | | TCTATCCTTTTGCA |
| MI05 | 4 | 10229304 | TCCATCATCATAGCTTTCGCACTTTGTAATCTCCC[C/T]GGTGATCTTTAGCCACCATCC |
| 76 | | | ATTGTCCACTAGCT |
| MI05 | 4 | 10797562 | GTCGTTGAATGCGTCGTCATAGCGGTTGGACCATC[A/G]AACTATCCAATGATAACGA |
| 77 | | | ATTCCAACTACAACGA |
| MI05 | 4 | 12922418 | GTTCAGGGAAAAACTGACCCAAAATCAGAGCGTTT[G/T]GCACTCAGATCAGCTTTTG |
| 78 | | | GCGGTCACAATCTAAA |
| MI05 | 4 | 13123231 | ACCATTAATTTCCCGACTTCTACATGTATTGTTCA[C/T]TTATGTAGGTAAGTCCACAAA |
| 79 | | | AAAAGTTCAAAATT |
| MI05 | 4 | 13816193 | CTCATGCTTCATATATGGTGCTCGACCTCCACATC[A/G]TTATCATGTAGGTGTGATAGT |
| 80 | | | GCCCAAAGCAGCGA |
| MI05 | 4 | 14527969 | ATGGCAAAGGTAGGAAGTAATTAGGGGTGAACGTG[A/C]ACTCACAAAATTTTACAA |
| 81 | | | AACCAGTGATTTATTAT |
| MI05 | 4 | 14884472 | TATTAACGTGATTGCCGTATTTTTCTTATTCTTTT[C/T]TAGTAGATTCGTATGCCATTT |
| 82 | | | ACACTTTTAGGACT |
| MI05 | 4 | 15819509 | GCCCACTCATCAGTCGTGCGTGTGTCTGCTCCCGC[A/G]CGCTCGCATGCTGGTTCTTT |
| 83 | | | TCTCGGCTGTTGATC |
| MI05 | 4 | 16892214 | TAGGAGGAACATATCCGATACCATATTGCGTAGTG[G/T]CCTCACAAGCAATGTGGAAC |
| 84 | | | TGTAGAGAGGTAATT |
| MI05 | 4 | 17181639 | TCCTAGCCGCGTCTGAGCAACCTAACCTACCCGCC[G/T]CTTGCTTTTTATAGGGCTGG |
| 85 | | | GGGACGTGTGGGAGA |
| MI05 | 4 | 17317457 | GGCGGACGTATGCCGCGCTGCTGTCCAGTGGGGCT[A/G]GGCCACATCGTGATTTGCC |
| 86 | | | CTGTCCATGGATTGTA |
| MI05 | 4 | 18762816 | ACAAGTATCGGGTGGCACTCTACTTGTTCTAAAAT[A/C]AAAAAAGGTAGAGCTTGCC |
| 87 | | | TGTGAACCGACCAGTA |
| MI05 | 4 | 18929490 | GAAAGGTTGCCTTGCCATGATGACTCTAGTAGCAG[C/T]CCTCACACGTTTCCACCTC |
| 88 | | | ACTAGTGCCATGCTCC |
| MI05 | 4 | 18938489 | AACGGAAACAATACCAGTGTTCGCTACAATACAAA[C/T]TGTTAAAAAAAGGAGGAA |
| 89 | | | AAATACAAGCAAGACTG |
| MI05 | 4 | 20883311 | TGTCCCCTTTGTTGGTTTGGCATGTTGTAGGTAGT[G/T]GGAGCAGGATACAATTTTGC |
| 90 | | | TTCACATGGGACGAT |
| MI05 | 4 | 22733202 | TGCCCTACAGTAAGGAACATTATGAGTTCAACTAT[A/C]AGTTGTCAATATTGTTGGTT |
| 91 | | | TCTGTTCTAGTCTGC |
| MI05 | 4 | 23215098 | TTAATTGTTTGCCCTTGTTAGGTCTGCGAGATAGC[A/G]GTCCCTTATCTTGCGCATCTC |
| 92 | | | GGCTGCGGCATCAC |
| MI05 | 4 | 23816593 | TTGCTGGGGAGGGAGGTGCGTGAGAATCTCTTCGA[C/T]GACGTTGCCACACTCTTCC |
| 93 | | | ACGACCTGTGCAACGG |
| MI05 | 4 | 24436803 | CTCATGATAAGTAGATGGTTCAACAATTACTGTAG[C/T]AGCCCGTGGAAAACCATAAC |
| 94 | | | GATCTGGAGGTTCAA |
| MI05 | 4 | 24663248 | GTTACAGATTCGAGCACTATTCTAAGTTGTAACTC[C/T]GATGTGCTGCTTTCGTGTGC |
| 95 | | | TCGTTCTTGTCTTGT |
| MI05 | 4 | 24688037 | TTCGGCGTAAATAAAAGTATCAAGTGCGGAGTAGC[A/G]AAGGGGGAGAAGCTAAAG |
| 96 | | | TCTTGTGCGGTAGGAAA |
| MI05 | 4 | 24925231 | TATTTTTGTTGGAGCTATCATTTTATCGTTGTTCT[A/G]GGATGCCAAATTCTCATGGTG |
| 97 | | | ATACTCACTAGTAT |
| MI05 | 4 | 26010181 | AAAATATATATATAAACGGGAGGACGATTGGACGA[A/G]AGGAAGGAGCACGTCCTTC |
| 98 | | | GTCCGATCCAAACACG |
| MI05 | 4 | 26745100 | TATCGTGTAGACCATTGGGTGCACTTAGACTTTTT[C/T]AAACTCGGGATGGTAAGCTC |
| 99 | | | CATATGGTAAAGTCA |
| MI06 | 4 | 26915339 | AGTACAGCCGGGTCACCCGTTTGTTTTCTCTTGCT[A/G]AGTGAAATTTGATGCTAGTT |
| 00 | | | AGAAATATTAAATAT |
| MI06 | 4 | 29078289 | AATTTGACCTTTTTTAACCGATCAATTACTCCTAG[A/G]CCAGCACACATTCTCCATCTA |
| 01 | | | GGGAAAACAGTGCA |
| MI06 | 4 | 29393688 | TGAAGGAGTCACAATATGAATTGTTATAACACAAG[C/T]CCTGGCCTCAAATATTTGTT |
| 02 | | | TATGAAACATGAATC |
| MI06 | 4 | 34568795 | TCTTGTGAGCCAATTAGAGGAAAAACATATGATCT[C/T]AAAACATTCAATACATGTTT |
| 03 | | | ACTTTAGCCGTTCAT |
| MI06 | 4 | 35853582 | GGACAAGGTCTGAAGGAGATGCTGGAGTACTACGA[C/T]AATGCGGTGAGTCCGTGA |
| 04 | | | TGGGGATTACCATTTGC |
| MI06 | 4 | 36062406 | TAATCCCATGCGGCTTGCTGCTTAGTTGGTACACC[A/C]TCCTGCGGTTTTAAACTAAT |
| 05 | | | ATAATGGCGCCAGTA |
| MI06 | 4 | 39828296 | GGTGGCGATTCATCGCCGGAGCCCCCTGGAACTGG[C/T]GAGCTGTTTGGTCGATCCG |
| 06 | | | CTGTGACTGTGATGCA |
| MI06 | 4 | 45694268 | GTGAGCCGCATACATGAGTAGGAGAACTGTCTCCA[A/G]TACGCGAGAGGTAAGAAG |
| 07 | | | CGTTTGAGATTTCAGTT |
| MI06 | 4 | 51011956 | GAACATTGTTTGATCCCGAAACTCAAAATCCTAGC[C/T]TTCTTAGGTAATATTTGCTTT |
| 08 | | | ATCCTAATGCAGTA |
| MI06 | 4 | 52769850 | CGTTTTAGAAATAACCTCTGAGTTATATCAAGCCA[C/T]GTCGGCGTTTTAGAAGTAAC |
| 09 | | | AGCCGAGTGATGACT |
| MI06 | 4 | 54904158 | GACAGCGCGACGTTCACTTCCTAACAGTAGAGCGC[A/G]AAGACAACAATAGTCCTC |
| 10 | | | ACCCAGTTAAAAACAAA |
| MI06 | 4 | 55263339 | GATTATATTATATATAAGGTAAGAAAAATTATTGA[A/C]AATTGCATATGTGGAAAAATA |
| 11 | | | GATGAAATATACCA |
| MI06 | 4 | 56168910 | CCACCATCGATCGTTACCTTAGTTAACTGCCGACG[A/G]CCCATTGTGGCCGTCGGAC |
| 12 | | | ATAAGCAAGATGTGTA |
| MI06 | 4 | 56337464 | AGCTAGGAGACATCCATGACCATGACCGGTGGTGT[C/T]CTGAAGAGACGCGTCTGA |
| 13 | | | AGAGAGGGGGGCCTTTT |
| MI06 | 4 | 56366009 | TTTTGGATCGATCAAAGCTACGGATTTTGGTCAGC[A/G]ATGCTTTACTAACATAACAT |
| 14 | | | CAATCTATCCAGATT |
| MI06 | 4 | 57836302 | TCACACGAACCATAGTCAGTAAACCATACAACCAC[C/T]GCAGGGAGCCATTTGTCCT |
| 15 | | | CACGTGATGACGTGAA |
| MI06 | 4 | 60018315 | CGACTGATGACTCGACCAGTAAGTAATAAACAATC[A/G]ATCTATGGACAGCAGGAAA |
| 16 | | | TTTCAGACAGTATCCC |
| MI06 | 4 | 61026968 | CGTATTTGCTCCCTGATCTTTACGGAGTACTTCCC[A/G]GGACCAGCCGTCAGTGGTAG |
| 17 | | | AGGCGCCCGTAGAGA |
| MI06 | 4 | 62266919 | ATTCCCACTCATACACAAAAACAGCTGCCAGTTGG[C/T]CATGGAAGATGAGAACGA |
| 18 | | | AAAGAAAAAGGAAAGCA |
| MI06 | 4 | 62813721 | TTTGTTTTTTTTTTCTTTACCGCAGTCAACCATTC[A/T]TATTTCATCAGTACATTGTCG |
| 19 | | | TTGTTTGATTCGAT |
| MI06 | 4 | 70395376 | CCACGTTAGCCTCAACGATGTCCACAAGCCAGTAA[C/T]GTGGTGGACTTACCGCGAC |
| 20 | | | CCCTTTATGGCGGTTC |
| MI06 | 4 | 75029980 | GAAGGAAGCCCATGACTCTGACTATTCCATTCATC[C/T]CGGTAGCACCAAGATGTATT |
| 21 | | | AGGATCTGAAACAGA |
| MI06 | 4 | 75342026 | ATGGAGTATCCCTACAATGAAATCATTGGTCAAGG[G/T]ACACTGAACGCCTTTGAAG |
| 22 | | | TGATACTCCACTCAAC |
| MI06 | 4 | 75909294 | TTTTGTCATTTCCATGAGTCCTTTAGATATTTCCA[G/T]TTTTAGAATTTGTACCCTTTA |
| 23 | | | GCAGGTGCTATTAC |
| MI06 | 4 | 77040035 | GTAGAAACCATGGGGGATATGCCCAATGTAGACGA[C/T]TGTGGCTGTGTTTTCGGGC |
| 24 | | | TCTTCAGGCTGCTGCA |
| MI06 | 4 | 77370172 | AAGAACGAACTGACCAGATGGTCAAGCTCGAAAGC[A/G]AACTTCTGCGTCTTGAGC |
| 25 | | | AGCTTGGCTTGCTTTTT |
| MI06 | 4 | 77387825 | GTCTCGTCCTCGAAGCCTCCTCCCTTGAGTCGCTG[A/C]GCAAAGAAGTCGTACTTCA |
| 26 | | | TAACATCTAGGTCGCT |
| MI06 | 4 | 78023503 | TAGAGTTGAAGAACGAGGATTGGTGGAATACGCTG[A/G]CGTAATTGTCCCTGCTCGA |
| 27 | | | TTGCCCCATCCCGCTG |
| MI06 | 4 | 79618987 | CGACTGGATACAATGATAATGCAAACTGACACGAG[C/T]GCAATGGACAGTGAAGCA |
| 28 | | | AAACAATACTTTAAGCT |
| MI06 | 4 | 82264820 | AACATGCATTTCTACGACACACCTGCGTCTGCCCG[G/T]GTGAGTGGGAGTGGAGAC |
| 29 | | | GGTATCTGCATTTAGAA |
| MI06 | 4 | 82387404 | GACGGGAACAAGAAGGACGACGAAGATGAAGGACA[A/G]ACCCTCCAGAAAGGAA |
| 30 | | | AAACAAAATTCTGCCACGT |
| MI06 | 4 | 82669634 | GGCCACCAAGCTGGCAGGTTCTAGCTAGGGTTTCG[C/T]CATAGATGGATGGATGGAT |
| 31 | | | CTACACGCGGTTAGCA |
| MI06 | 4 | 87409783 | TGGAATGGAAGCCTCCTACTACGGTCTGGCAGATT[C/T]AGAGTTTTCTAGCATTAGCT |
| 32 | | | GGATATTATCGACGA |
| MI06 | 4 | 94089079 | AGAAAACTTTCATAGGCCAAAATAATTTGGATTAA[C/T]TGTATACATGCCACTCAGCT |
| 33 | | | TTTGTAAAAGTAAAT |
| MI06 | 4 | 96567536 | GGCCACACCAACAATGTCAGGAGAATAAGGTGAGC[C/T]CACTATACATGTCGCGACA |
| 34 | | | ATAGAAGCTTCCGTTG |
| MI06 | 4 | 11047004 | AATCAAGGTAAGGCTAAGCAATAACCAAGGTCTCA[C/T]TTTGTAGAAAAACTTACTA |
| 35 | | 2 | ATAGTGGATCCTTAAG |
| MI06 | 4 | 11872331 | GGTACTTTGGTTTTGGAGACTGCTACTGACTGCTG[A/C]CGTCGTCGGGCCGCTCCTC |
| 36 | | 4 | TCACCTCTGGTAAATA |
| MI06 | 4 | 11943091 | GAGGCGGTCTTGATTGCTCGCACTGCGATGACGTG[A/G]GACTTGATCCTGTTGGGGA |
| 37 | | 3 | CTTGTTCTCAAATGCT |
| MI06 | 4 | 11972150 | GCAACACTTTCCACTGAGCTGGGGAGGCAAAGAGA[A/G]AGAGAGCTTAGCCGCAA |
| 38 | | 3 | GGCGAAGCAGCGGCGGTA |
| MI06 | 4 | 12277321 | GGGAGTTACGGATCCGGGCGAAAAGAGTTGGTGGC[C/T]AGGACGGGCGCTGTCTGG |
| 39 | | 2 | TGGAGAAGCTCGGCCAT |
| MI06 | 4 | 12455306 | CTTCCCATTTTATTTTCCATCGTTTCATTTTTTTC[G/T]TCTCTTGTTCAGTCATGTGCC |
| 40 | | 7 | GCGCCTCTTTAATG |
| MI06 | 4 | 12597447 | AACATATAGTTAAGGAAAATCATTAATGTTGTATT[C/T]CCTTTATTATTCTTCCCTTGA |
| 41 | | 9 | ATACAAGTGATTAC |
| MI06 | 4 | 12597674 | ACATCGTCATATGCTACCTCACATGGGCTCCCACC[A/G]GTTACCAAGAACTTTGTTTT |
| 42 | | 4 | GCTCAAGTTCCCATA |
| MI06 | 4 | 12689392 | CTCCCACATAGTACTGCATCTCCACTATCGGAATC[C/T]GGCTCTTTGCCGAATGCCAA |
| 43 | | 9 | GTGATTCAGACACTC |
| MI06 | 4 | 12788108 | AATCATGATTCAGTTTTGTTTTTTTGACTTTGGTG[C/T]GAACCCCCTAACAAGTTGAA |
| 44 | | 6 | AGATCTAGCATGGGC |
| MI06 | 4 | 12893982 | ACTGGAGTTCGGTGGTGCGAACGCGACAACGGCGG[C/T]GGTCATGCCGGCGTGCAT |
| 45 | | 4 | GTGATTCGGTGATTCCA |
| MI06 | 4 | 12952576 | CTACTTATATGCACTTGGGCAAGGCACTGGGCAGA[C/G]TACGCAGCCCGGGATCTCG |
| 46 | | 9 | GGATTTTTTTGTTACT |
| MI06 | 4 | 13006743 | AGGATTTTCATCGGCATGACTTGGTTTATGAGGGT[C/T]TGACGTCACCGGCACATGTG |
| 47 | | 5 | GTATGGCAACGCTAA |
| MI06 | 4 | 13030816 | CTTAGTTTGGGCCATAACTCAAATCCTGATCAAAG[A/G]TTAAGCAAAAGGTGAGTTT |
| 48 | | 2 | CCTAGACTTAGTCAAT |
| MI06 | 4 | 14098925 | ACTAAAATTAGAAGCTAGAGTCAGGCACATATACC[A/G]TGTGCCCCGTCATGGGTAC |
| 49 | | 1 | AGAGAAGATTTTTTCG |
| MI06 | 4 | 14601491 | ACCTGGTCAAATAGGCTCAAGAGGTCTTAATGTCA[C/T]GTTTACTATTTGCGGGTCAA |
| 50 | | 4 | AGCTTACCTCGGTTT |
| MI06 | 4 | 14826007 | GTAGTTGTAGTCCGTATTGTGCTGATAAGTTTTTG[C/T]GTACTATTGATGATAATATAC |
| 51 | | 4 | TTGAATGGAGCACA |
| MI06 | 4 | 14980349 | TTTCTTTAGAAGAAGTCTAGAAATTTCGACATTTA[A/G]GTGAGAAGGAATCTCTCTTG |
| 52 | | 6 | ACAAATTTTTTTTGA |
| MI06 | 4 | 15532219 | AGCGATGGTGATCACCTTAGTTAAGCTCGATCGTA[C/T]GGTACGGATACATCAAATGT |
| 53 | | 2 | GTGTGGGGGGTACCA |
| MI06 | 4 | 15714029 | TCTGTGCAACAGATTAACTAGCTGCATTGTTAACT[G/T]TTAGTGCCATGTATATTTTTC |
| 54 | | 5 | TCTACGGCTAGAAC |
| MI06 | 4 | 15781098 | AGACAATATTCTGTTGGCTCTGACTTGCCAGCCGC[C/G]GCAGTCGTGCCCTATCTATG |
| 55 | | 4 | GTGCCACCTTTAACT |
| MI06 | 4 | 15809797 | AGCTCCTCGACAAGGTACGTACACAGATTTGTATA[C/T]ATCGAGCATCTAGCTAGCTT |
| 56 | | 3 | TCTAGTATGTTCAGA |
| MI06 | 4 | 15920083 | CTGTACACAGGTAATAAGGAGATGTGCATTTCAGT[C/T]TTTATGTACTTCATTTTATGT |
| 57 | | 4 | ATTTAGAATCTATT |
| MI06 | 4 | 16367468 | TTCCCCATACCTAAAACATTCACCCATGGCCGCGA[C/T]CCCTTCATCCTCCTGAATCC |
| 58 | | 7 | CGGCCGCCGCTTCTT |
| MI06 | 4 | 16392653 | CCCATAGATCAAACAAGCAGTCTTGTTGAAGAAAA[C/T]CCCCCTGGACAATCCTCTT |
| 59 | | 2 | CAATTGCTGATGCGTG |
| MI06 | 4 | 16420104 | AAAGTGACCGATGATCATATATGCAGATGCCTGAG[A/C]CTGTGAGTTGAGGTGGAGT |
| 60 | | 8 | CTGTTCAAACGAAATT |
| MI06 | 4 | 16911433 | CCTATAAAAGGGCCGCCCTGAGCCCATCATCTCCT[C/T]GCACCGGCCTCGGCCACTC |
| 61 | | 0 | TCCTCCCCCTCCCCTG |
| MI06 | 4 | 16999430 | AGTGTATAAATATACAGTGAAACAGCTTGCACTGG[A/G]GACACCACTTCCCAACGCA |
| 62 | | 2 | AGCTCTTCCAAAAATT |
| MI06 | 4 | 17027675 | ACAGAAGGCATAATCAATTATCTATCTAGTAAACC[A/C]TTTTGGTTTGCATGCTGCCAT |
| 63 | | 6 | ACAAAAAATGGTAA |
| MI06 | 4 | 17180224 | AAATAACGGGACCGCAGGCGATTGTTAATTCAGAC[A/C]GTACATGATCTTGATTATAG |
| 64 | | 9 | CCATATATATGAAAC |
| MI06 | 4 | 17286526 | ACAATACAGATGAGCTAATATTAAACATGATCAGA[C/T]GGTAGGATTAAAACTGACAT |
| 65 | | 2 | ACCTTTCCCATTTGT |
| MI06 | 4 | 17540498 | AGGGGATCAGAGGATGGAGTTATCCAAATGTAGGC[G/T]GGTACAACTGAAGAAACA |
| 66 | | 2 | GGAGCCGGGAAGGCGAA |
| MI06 | 4 | 17767392 | CTAACCCTTCTCCTGTTTCAAATCGTTTCAGACGT[A/G]AGAAACCAGCCGGTCCTCA |
| 67 | | 8 | TCCACTGCAAGAGAGG |
| MI06 | 4 | 17862434 | AGGCGCTCGATAACAATAGGCGATGTGGCACTAAA[C/T]GGCAAACAGACACACTAC |
| 68 | | 8 | CGCTTCTATCCGCCTGG |
| MI06 | 4 | 18108678 | AATGAAAACATTAAACATGAATTCATGGAGCACCT[C/G]TCTTGTTGCTCCACTATATT |
| 69 | | 4 | GGTAGAGTTAGGGTA |
| MI06 | 4 | 18145405 | CCAGGTGGTGTAATTACAATGCATGTGGTAGTGCG[A/G]CCTCCACAAGCTGACAAAA |
| 70 | | 0 | ACAGTGGTATTGCCGA |
| MI06 | 4 | 18321342 | TGCCGGAGAAATCCGTGCAGGAGCAGAACTCAGTC[G/T]CATCATCTTCCTCCACCCG |
| 71 | | 6 | ACAGATGATTGGAGGA |
| MI06 | 4 | 18604399 | GTGGAGAGTTCATCGCCATGGAGTTCACCACCTAC[C/T]GCTCTGACAAAGGTATTCA |
| 72 | | 8 | GCGGCACTTCTCCGCT |
| MI06 | 4 | 18744259 | TATGCGGGCGGCCTTGTTGAAAACACGGTAGACAT[A/G]CTTGACCGATCCATAACCA |
| 73 | | 0 | AGAAGAAAACATTCAT |
| MI06 | 4 | 18806297 | CCTTAGGATTTTCAGAAAACTGGCAGGTATGAAAC[C/T]CAATTCTGATAATAGAGTCA |
| 74 | | 5 | AAATATCTTGAGTAT |
| MI06 | 4 | 18823446 | GTGCTTCCAACTTGATAAGGTAGTAGCTCCATGGC[A/G]GTGACTATATGGGAAGAATC |
| 75 | | 6 | AAAAGAGTTCCTTAG |
| MI06 | 4 | 18867299 | ACTTTTGTCATGAAGCAACAGGAATCCCAGGCTGC[A/G]GTCTTATCAGTTCATCACAT |
| 76 | | 7 | TGGAGTCCCCTGTGT |
| MI06 | 4 | 18951701 | CATCACAGTGAGGATCTGGCAGAGGCGGCTTTCAG[A/G]GAATCAGAGGTAAAGGAT |
| 77 | | 3 | CCGATCCACCGCGCATC |
| MI06 | 4 | 19007553 | TCATCTCTCTGGAGCTACTTACTAGGCGGCGGCTA[G/T]AGCTAGGCGTATCTCTTCTT |
| 78 | | 4 | CGTGCGCGATGGTGG |
| MI06 | 4 | 19007870 | AGTTTCCACTAGATTATTCTTGAGAAACACTCATG[C/T]CCATGTGGTCGAGCCCTAAT |
| 79 | | 3 | CCTTGGTTCTTGATT |
| MI06 | 4 | 19115395 | AAATATAAAAGAATTGGGCGGGACCTAATTGTATT[C/T]TGTATCACGTTTTCTACCTCT |
| 80 | | 0 | TTGCATCGTTGATA |
| MI06 | 4 | 19283667 | CCTCAGGATGTTGCGACTACACTGTGACGACGTTG[C/T]CACTCACAATACGCCGATC |
| 81 | | 4 | AAGACTCCCTCCATTA |
| MI06 | 4 | 19343564 | CCTGTGAATTAAAGTCACATGCATTCTATCACATG[A/G]TGTTAATTATCCAAAATAAA |
| 82 | | 6 | AAGCAATCCCAATGG |
| MI06 | 4 | 19378743 | AGGGAGGCGAGTTCTTTGATTCTATCGATTTGGCC[A/G]TGCAAGTGCCGGAAATCTG |
| 83 | | 4 | ATGTAGGTGTACACCT |
| MI06 | 4 | 19512304 | CCCCTGAGTCTATGCTTAATAGAAAGACCAATCAC[C/T]GCGATATTTTATTTTGTCCGT |
| 84 | | 9 | TTGGAGTCTAAGTA |
| MI06 | 4 | 19550641 | ACTTTATAACATATATGTAGTCAAATGAAAAGGTC[A/G]TATGTGTATTGAACCCAATCA |
| 85 | | 1 | AGCTAGTTTCGAAT |
| MI06 | 4 | 19642108 | GGAATTTGGCACGGGATTGAAAACGGGTCTCACGA[A/G]CTAATAAAAAATGATCAA |
| 86 | | 6 | ATTTATGGGCGCGTTAG |
| MI06 | 4 | 19971934 | AGAGGCAGATCCAGACGGACACTTTGATAATTTAT[C/T]ATTGTGGAAACAACATAAA |
| 87 | | 1 | GCTATGGAACTTATCT |
| MI06 | 4 | 20102536 | ACAATAATAATTAGTCGATAACGATGCAATTTAGG[A/C]AAATACAACAAATTCACCTT |
| 88 | | 2 | AAAAGACCTTATCTT |
| MI06 | 4 | 20198832 | AAGCACTTGCCTCCTTCCCTTAGAAGCCTCTCTAA[C/T]GTCGCGCCTCTGTGTCTGCC |
| 89 | | 6 | TAGCTTGTTCCCTGC |
| MI06 | 4 | 20240083 | ATTAAATTCGGTCCCGTATGCATGCATCGATCGGC[A/G]CCTAGATCGGGGGATGTTGC |
| 90 | | 3 | CAGTACCATAGATGA |
| MI06 | 4 | 20246428 | TGCTCCGCTGAGATGAGGGATTTCCACGCTTATCC[G/T]TGTATGTCTATTGTCTAGCCG |
| 91 | | 0 | ATGACGAGGAGGTA |
| MI06 | 4 | 20287966 | CAAAATCAACGTGAACACATAATCGGCAGAATCGT[C/T]GCGATAGGAGTCCGTCATT |
| 92 | | 5 | TCCTATATCCTAAACC |
| MI06 | 4 | 20351610 | GTAGTTTGCTTGTGTCGTTGGAATTTCTCATCCGA[A/G]CCGAGCCTCCGTATCACCTC |
| 93 | | 4 | CGCACATTGCCATGG |
| MI06 | 4 | 20355378 | ACTGATCTTTCTAGATCGATGACATATTATTCTTC[A/C]TCATCAGATGTACCAAACTTC |
| 94 | | 3 | AGAGCTGGAGGTCG |
| MI06 | 4 | 20382449 | ACAGTAACTTCAAGGCTCGATGGGACATGGAGGGA[A/C]TGCAGAAGGATCTACAGC |
| 95 | | 2 | ATGTTCTGGAGATACGT |
| MI06 | 4 | 20451767 | ATAATTTCAATCACGCATTATAGTTGATAGACTGG[A/G]CCGAAGGTGGATACTTAACA |
| 96 | | 0 | ACCGATTCACCAATG |
| MI06 | 4 | 20609718 | TATTCCTTTTCTCTTCATACAATACAAATTTACAG[G/T]CATACCTTCGGCTCCTTGACA |
| 97 | | 7 | CAAGATAACACAAA |
| MI06 | 4 | 20725827 | GTCCCACACGATGAGGTTCTTGTCCTTGACGATCA[A/C]CTCCACGTTGTCCGCCTGC |
| 98 | | 9 | AGCTGATCCTTGGAGA |
| MI06 | 4 | 21093585 | TTCACTATCACATGTGAACCTTGAGGACTGCTCTT[C/T]TCAGAGAGCAAAGAATTCG |
| 99 | | 2 | GCGAATGTGTGCTTTC |
| MI07 | 4 | 21206505 | TTCGTAGCGTCATCTAGAGAAGTGCTACCTTTGGT[A/G]GTCACTTTCTTAGGGGCCAT |
| 00 | | 5 | CGCAGGTTTGGGTTG |
| MI07 | 4 | 21238398 | AATGTGAGCACATAAATGGATACCAAACTCAATCA[A/C]ACTCACTGTGTGCGATTCA |
| 01 | | 7 | AGATCACTAGCGACTA |
| MI07 | 4 | 21430519 | GCAAGCCCATATAGTCTGCCACGGATTGCATCATG[C/T]CGTCCTTCTGAAGAAGCAGT |
| 02 | | 8 | TGTGCTGCACCACTG |
| MI07 | 4 | 21831654 | AAACCTTTTTTTGACTTTGGCACGATGGAAGTCGC[C/T]GAAGCAGCAATCTTTCTTTT |
| 03 | | 2 | CTTTCCTTGCTTTCG |
| MI07 | 4 | 21906989 | TTTATAAATCATGAGAAAAGGTATTAAAGCAATAA[C/T]CAATTCAACTTTAATATGAGT |
| 04 | | 9 | TACATTTTGGTTAG |
| MI07 | 4 | 22350112 | GATGAGCAAGGTATTGATCCATCTTGGGCTGCTGT[A/G]GTTACGCCGGTTTTACGAGT |
| 05 | | 8 | CAAGACAATTTATAT |
| MI07 | 4 | 22579962 | TGATCACAACTGCTTGTATTTTTGGAAAATATCCA[A/G]TCAAACTTTGAAACTTTGAC |
| 06 | | 6 | CGCCAATAACTTTTA |
| MI07 | 4 | 22608528 | TGTCAAATCACGTGGACTAGCCTACGTGGTTTGCG[C/T]CGATCGGCTCCTTGAGCTG |
| 07 | | 1 | TTGGTCGTTTCTTTTA |
| MI07 | 4 | 22651897 | CACCATTCAAAAACTGATTTATATATTATGCAATG[C/T]GCTATAAGAGAAGGTTAAGA |
| 08 | | 9 | CATCCCCATCGAGAT |
| MI07 | 4 | 22679090 | TGTTTTCTTTTCTTTTCTGGATATCTATCCATCCA[C/T]AGACACGCGAAATGCATTCCA |
| 09 | | 3 | CAGTTGTCCTATGT |
| MI07 | 4 | 22726989 | TTCATGTGATGGATCTGACCGATCATTGGTTGGTC[C/T]AGATGATGGATATTGAGTGAT |
| 10 | | 6 | AAATAGCTCAGTGC |
| MI07 | 4 | 22774979 | CAATTGGTACATATGCATTGCGAGTTCTGAGATCA[A/C]ATAAGTAAACAATACATAAT |
| 11 | | 5 | CAAATGGGCTGCTTT |
| MI07 | 4 | 22796464 | TTAATATTAGATGATTTAACTCACTCCTATCCCTC[C/T]CAATCATCTCTATCCCAACAA |
| 12 | | 5 | GCTTCGAAGGGATT |
| MI07 | 4 | 22799801 | AGCAACTTGTCATTGGCAAGGATGATGTGACCATT[A/C]TGGTTCTTAGACTAAGCCCT |
| 13 | | 0 | CGTGTGCTTCTTGAA |
| MI07 | 4 | 22934998 | TACTTATCATGCTTCATTTGTAAACTGAACATTGT[C/T]GTTTTAAATAATTCTATCACT |
| 14 | | 3 | TCTACATGTATGTA |
| MI07 | 4 | 22935067 | TGTGCACCCTAATATTCTAGCAATGGTGACTAGGC[A/G]TATTAACAAATATATAAAGC |
| 15 | | 2 | ACTATCTCTGGAGGG |
| MI07 | 4 | 23117441 | GATGCATTTTAATTGTTGAAAGGGTCATGATGCCT[A/C]AAAAGAGGGTGAATTAGGC |
| 16 | | 3 | TTATCTAAAGTTTCTT |
| MI07 | 4 | 23175237 | CTATTGCAGGTTGCTGACTGCTATAGAGTACATGA[C/T]ATCCATTTTTTCCCCAACGGT |
| 17 | | 6 | ATTAATCAACACTT |
| MI07 | 4 | 23186292 | CGATTTTGGAGAGGAGTGGCACTGCTGCCACATCA[G/T]ATAGGGAGTGGTACGACCT |
| 18 | | 1 | AGGATGCTCGAAGACT |
| MI07 | 4 | 23303688 | TGCATTCTGGCCACAACTCGATCTAGGGATGAATG[C/T]GGCCATCGACAGATGGGAT |
| 19 | | 0 | CTCGAGGCAATGGAGG |
| MI07 | 4 | 23347075 | CGAGGAATTCGTCATGGAGGAGGATAATGAGGAGT[C/T]CCCCGCGGATCGGTGCGTC |
| 20 | | 0 | GACAGGGTCCTAGAGG |
| MI07 | 4 | 23348135 | GATGTCTCTGGAAGAGTTCATCAGTACCAATTCAT[A/G]GTGAACATATTCATTTGGTA |
| 21 | | 5 | TATGCTAATACGCTA |
| MI07 | 4 | 23639153 | GGGAGATGCAGTCATAGCTCGTTCGAGGCCACCAT[C/T]GGGCGGGAGACGCAGTATC |
| 22 | | 3 | TTATGACTGATATTTA |
| MI07 | 4 | 23695995 | GTGCGTCCACTCTAACAACAACCTACAAGAACCCA[C/T]TATATGTATTATGGGGATAT |
| 23 | | 4 | CTAAAATGCTTGCAA |
| MI07 | 4 | 23778886 | GATTGGTTGCTCCACCCAATACATCCAGGCCCACC[A/G]CGTTCAGTTGACCGTGCAG |
| 24 | | 4 | TGCATGTGAATGGTTA |
| MI07 | 4 | 23815236 | TCTAGAACGACTGCTACTGTATTTTGGCAGATGTG[C/T]GTTGCATATTGCTACTGTATT |
| 25 | | 9 | TTGGAGGAGATTTT |
| MI07 | 4 | 23821987 | TTCAACAGGACACGATGGGCCTGAGCGCACCTGGC[C/T]GAGTAACCAATCACGCCT |
| 26 | | 3 | AAAGCTTCCAAAAATGT |
| MI07 | 4 | 23886345 | TTACCTTCATTTAAGCAGATCACCTTTTGCCAGAA[C/T]CATTCCAGAACAAGTAGACC |
| 27 | | 7 | GGAAACCGGATTAAG |
| MI07 | 4 | 23986892 | GACTAGCCGAGAAGGAAATGGAACGTGGCCGACTA[A/G]CCGAAAATGCGTCGTGGC |
| 28 | | 6 | CGCCCGCACCGGGAAGA |
| MI07 | 4 | 24003209 | CATGATAATTTAAGTAGGTGGTGGAATGACTTTAA[C/T]ATAGGGAAGAGGAGCTGGC |
| 29 | | 1 | ATCCAAGTGATGTCGT |
| MI07 | 4 | 24078533 | TGGTCTAACTCTTCATCGATGCATCTAACGGCCAC[A/G]AGCTCATCACGATTTTGCTT |
| 30 | | 9 | GAACTCAACGTAAGA |
| MI07 | 5 | 657550 | CTGGATCGGATTATATATCGTCTCGGCAGCTAGGG[C/T]GGCCAGCACCAGCCATGACT |
| 31 | | | CCTTCGCTTCTCCTC |
| MI07 | 5 | 1934320 | CTGGAGTCGGTTGATAATTACAGGCACATTGACTG[C/T]CCCCTTTACCTTTCTGCAGG |
| 32 | | | CGAGGTCGTACTCCT |
| MI07 | 5 | 2787807 | CAAGGTTAAACCCTATCCATTTATTCTACCCCAAC[C/T]ACAAGTCCACGACTAGCACC |
| 33 | | | AAGGTGTGAGACCCA |
| MI07 | 5 | 3992391 | CCCGATGATCCTGAGGTTCTGTGAATAGTTGTTAA[C/T]GCTGTGTTCTGATTTCAGGA |
| 34 | | | TGTACTTTTGCCGGA |
| MI07 | 5 | 4590005 | CTTAAGCAACTTGCATTTTTTTTATGTTGATCTAA[C/T]GATGCAAAGATTTTCTGCATG |
| 35 | | | CTTAGAGGGGAAAG |
| MI07 | 5 | 5642648 | TAGCAGCTTACGTTTACTTGTATAAGCAAATGAGA[A/G]GCACCAACAAACCAAAGTA |
| 36 | | | AGTCTGAATATATAAG |
| MI07 | 5 | 5797951 | TCTTATCTCACTTAAAGTTAGTTGAAAAAAATGCT[A/C]AGATAATGTGTCAAAAAGGT |
| 37 | | | GAAAGCCCATTTTTT |
| MI07 | 5 | 6334549 | CAGTCGCTGGGGGCAGTGGTATGAGGGTAAGGAGG[A/G]AGTTGGATGATTCAGTGG |
| 38 | | | TGTCATGCTATGATTCT |
| MI07 | 5 | 6375851 | TTCTGTCGCGGGAGCTATCCGTGCAAGTTCGTGCA[C/T]GATGAACGGCGCACCGAAT |
| 39 | | | TCACGAGACAGCTAGC |
| MI07 | 5 | 6931873 | CTGCACGAGTTTGAGGCAGAGGCTGCTTCTCCCCC[A/G]CAATTGGCGTGGCAAGGC |
| 40 | | | GGAGGCGGCATGTTGTC |
| MI07 | 5 | 7734869 | CGACGTACAGAAAATTACAAAAGAGATGTTTATCT[A/G]TGAGTCGAAACTACCTAGG |
| 41 | | | AGAATATTTCGCCAGT |
| MI07 | 5 | 10509121 | AAGAATCGCATTATTTGTCACATTTACGTGTGGAG[C/T]CTGAGTGGCCCATATGGCAG |
| 42 | | | ACAATACATCGGGCC |
| MI07 | 5 | 11402706 | GAGAGGAGAGATCGCGCAGAACTTTCGGATCGATC[A/G]GGCGCTCACCTTGGTGAC |
| 43 | | | GTACTCCATCTCGGCGA |
| MI07 | 5 | 11920659 | CATCGTACTGCTCCACCACTTACCCATACTTTTTT[C/T]GCTCTAAGTATCTTCTGGCCA |
| 44 | | | GTAAATTACCTCAG |
| MI07 | 5 | 15213441 | AACTGAGAAGTATGGCTTTTATGATTGATCCTTGA[C/T]TCCACTATGCTAACCCAACA |
| 45 | | | AGTGCCTAGTAAATT |
| MI07 | 5 | 16320899 | GCTCGCGATTGACTTGCAACATCCGAGTCCAACAG[C/T]GATGGAGCCTGAAAAAATT |
| 46 | | | AGGACCAAACCGTTAC |
| MI07 | 5 | 16667125 | ACGACTTGTTCAGTTTATTTTACGCGCGTGGGTTT[C/T]GGGTCCCCCTAACCTTGTTT |
| 47 | | | GTTTCGTGGATTTTG |
| MI07 | 5 | 17646466 | GACCGTGTCCCGTTTAATTATTTGTCCCATTCCAA[A/C]CTATGTGTGAACAACACAAA |
| 48 | | | TATAAAGGAAGGACA |
| MI07 | 5 | 19548622 | TTTCAGAGGCCGGGTTTCCTGGCAGGATACTGGAA[A/G]TAGCGGACCCGAACCTATG |
| 49 | | | GGCACACCTCCCTGAC |
| MI07 | 5 | 20034967 | CTCTCTATTTCCTCGCCCAGTTCTAAGATCTGAAG[C/T]CGTAGAGGGTTTCAGGCGAC |
| 50 | | | AACCGCATCCCACAG |
| MI07 | 5 | 20218664 | CATTGTCCTCACACGAGCACATGGATGATCACACA[A/G]CTTAGCACAGATGTCTTGT |
| 51 | | | GAGTGAAAACAGCAGT |
| MI07 | 5 | 22377052 | TGGAGACTTGAGAGTAAGATGGGCCCACGTGTCAG[A/G]AACCCCCCATCATGCCTAC |
| 52 | | | CCACTGCTGCATAAAT |
| MI07 | 5 | 22649947 | GGCACAAGATACTTTGTGTCAACTACTTGCGATTT[G/T]GTGCAAGACTAGCACAGGA |
| 53 | | | ACATGGAGTTTCTTCA |
| MI07 | 5 | 24882341 | ACAGTGACATCAAGCATCAGACAGGGAACCAGTGA[A/C]AAACAATGTACAACCTAC |
| 54 | | | AATGGCTAGGCTAGCGA |
| MI07 | 5 | 24971178 | AACAATCATTCCCTAACAGACCGAAAAGGAAGAAC[A/G]TTGCAGCCCTCTCTCAAA |
| 55 | | | ATTTGTTGCTACTTTAC |
| MI07 | 5 | 25417090 | ATTGTTGCGTTGTCATGGGCACTATTGCAGTCGAC[C/T]GTTGCGCTGGTAGCCGTTAC |
| 56 | | | TCTGCTGGGGCACCG |
| MI07 | 5 | 25817476 | GTTAAAAGAAGCCCGACCCTTTTTTATGCCTCGTT[C/T]GGTCGCTCATCATTCTTTAG |
| 57 | | | CTAACGAGGTGGTAT |
| MI07 | 5 | 27877780 | GGACGAAACCCTAGCGGTAATACCGAAGCTGTAAG[A/G]ATTTTCTAATTGGCAGTTA |
| 58 | | | TTGCGGGGTCTGTTGT |
| MI07 | 5 | 30431630 | CTGGCCTTGTTTCGGTATGCATATGCATCGTGGGT[A/G]GTACGGTTGGCTTTCTGACA |
| 59 | | | GCGAGATGAAGCTGC |
| MI07 | 5 | 30969873 | ATAATCAAATTTTTTAATCATTTGAACTGTGGCTG[G/T]TTGAATACTTGAATCTGTTAA |
| 60 | | | TTACTTTATGAATA |
| MI07 | 5 | 31132223 | GTAATCAATGTAAATGTAGAAGCTAAAGAGCAGAC[A/G]TAGAGATTGCAAACCCTTG |
| 61 | | | TGGATGCACTGATATT |
| MI07 | 5 | 33076387 | TTATGTGTTTAGTATTTAAATGCTTCTCCAACTGT[A/G]GCTATTGAAAATTTAGTTTTG |
| 62 | | | TTCTAACCAAAAAT |
| MI07 | 5 | 33461461 | CATAACAGTGTTATCTCAGCCCATAAAAGACGTTC[C/T]TAGTCTTACACCATTGCCTC |
| 63 | | | TTTTATTATCGATTG |
| MI07 | 5 | 34265898 | TTGCTGAAGAAAATGTCTTCATTGAGATCAGGTAA[C/G]TCAGCAGTTCCTGCATTTTT |
| 64 | | | TCCTACTCAGTTACT |
| MI07 | 5 | 34981843 | GGAAGCTTCTCGCTGGGGAGAGCCGGGACAAGCTA[A/G]AGCGACGGACATGGGTC |
| 65 | | | ACCAGTAAGCCACGGTCA |
| MI07 | 5 | 36698978 | GCACGCTTGCACCTAGGGACTGCTTGCATAACAGT[C/T]GTTGCACTATATTCTGATGC |
| 66 | | | GAAGATTTTTTTTTC |
| MI07 | 5 | 37085181 | GGAGTTGTCATGGGAGTTGGAACCACCTCCGAACT[C/T]AGGTGAGCGATTCCGAGA |
| 67 | | | GTTTCGTATCGAATCTG |
| MI07 | 5 | 39691300 | ATGTTCGCATAACGATAGCTACAGAGCAACAACAA[A/C]AAAAAAACGTTCCGATACA |
| 68 | | | TTGGCTTGATGTCATC |
| MI07 | 5 | 39897868 | AGCAAGCCATCAACTGGCTTAGTGACCATACTTTG[G/T]AACTCCTTCAAAGGTGAGG |
| 69 | | | AAATTTCACAAGATAC |
| MI07 | 5 | 40552455 | GGAGATATGTAATATTAAGTGGATAGAAGTGTTTC[A/G]ACAATTGTCAAAATTGCTTG |
| 70 | | | ACCGCTCGATCAATC |
| MI07 | 5 | 40670926 | CACGGCGACGAGGTACCACTCCGTTTTCGTACCAT[G/T]GGCAATATCATCGGTGCATC |
| 71 | | | TTCCCTGTCTGGGTA |
| MI07 | 5 | 42489567 | TTGGATCTTTTGGGCGTCTGGACACCCAGGCGACG[C/T]CTTTAAAACACTAGCCATA |
| 72 | | | GCGATAGATACACACA |
| MI07 | 5 | 42982239 | CATTTTGTGATCTGTTTTAACTTCTATCCTTCTTT[A/G]GCGAGGTTGATATATCCCTTC |
| 73 | | | TCAGTCTTGGTGTC |
| MI07 | 5 | 43772715 | TTTTCGGATAACATAAGGTTGGTTCTGCAAAGTCT[A/T]GGGCTTCGTTGTAATTAATT |
| 74 | | | ATTACCAATAAACTA |
| MI07 | 5 | 46832126 | TTAGGACGGAGGGACTAGAAGGAAAGATATATTGA[A/G]AGAAGACAAATGTCTATTA |
| 75 | | | AAAATAATAATGATAA |
| MI07 | 5 | 47148794 | GCAAAAGCCAGAGGCCGAGCTCACCAAAATGAGCT[A/G]CTCGTTGGGGAATTAAAA |
| 76 | | | AAATGAGAGGAGTAGAT |
| MI07 | 5 | 48904074 | TAGGAATGAACGAAAATGACGTGTGTAGTACGTGC[A/G]GTGCTTGAATACGATATGG |
| 77 | | | GAACAAAATCCAACTT |
| MI07 | 5 | 53459003 | GCTCACTTATGCTAGAGCCATTATTATAGTCCCCA[G/T]TAACAATTCTTTTGATTGTGG |
| 78 | | | TTCCAGCATATACC |
| MI07 | 5 | 53656341 | GAGTGAATAAAGTATTAAAAACACCTATAGATATC[C/T]ACTATACATACAAAATTGCA |
| 79 | | | AAGTATTTACATTAG |
| MI07 | 5 | 53901704 | TTATCTGCGTCGAGGGTCCTTGAATCGTGGCTGGC[A/G]AAGTCCTTTGTCTTGGCGC |
| 80 | | | TTGTCCCGTGCATGGG |
| MI07 | 5 | 55425948 | AACCCAAGTAGCCGCGCCGTCGTGGATGGAGAGAC[A/G]AGGGTGCGGTTCATGAAT |
| 81 | | | CATGAGTCCTGCTCCTG |
| MI07 | 5 | 55548348 | AATTTTACATATTGGTCATTCCTCCATTAATACTG[C/T]TGATCGCTCACTTGCTTTGTG |
| 82 | | | TCACGTTCTTCATG |
| MI07 | 5 | 56047845 | GTCGAGGTATCACTACATGTATCGGAACCGTCAGG[C/T]GGTCTCTTGTGTAAATATGC |
| 83 | | | GCTGAATAAGATGTG |
| MI07 | 5 | 57235134 | ACTAGTTTTGTAAAGTTTATATGCAGGCAAGGAAG[C/T]AGGGAGTGTGCTTTCTAGC |
| 84 | | | ATTTACGGTTTTGAAA |
| MI07 | 5 | 58621043 | ATTAGATGGGGCAAGAGAGTAGGATGCTAGGAGAT[A/C]TACGTGAAGACCAAGGGA |
| 85 | | | AGATATATGACCATTGC |
| MI07 | 5 | 60169328 | TTTTCCTTAAGAAGTTTTTTCCCATACTAAACACT[A/G]TGTCAACCACTACGATATGG |
| 86 | | | ACAACCCCTCCCCAG |
| MI07 | 5 | 60544505 | GTATTGGGTTTTAGTGATCGAGTGCATATGTAAGC[C/T]GGATACAGGGAAAAGAAAT |
| 87 | | | GAACAGCATAGAGAGC |
| MI07 | 5 | 61827491 | AACGTTATCCACAGATCTCAGCCGTCCGATCTAAT[A/G]CCGACGCTCCAGATTAAATC |
| 88 | | | ATCACTCTCGCGAAC |
| MI07 | 5 | 62623495 | GGGATGTTCACAGCTCCTTCAGGGTGGCCGGCGCT[A/G]AACTCGCCCTCCGTTCTAC |
| 89 | | | ACACAGACCAAATCGA |
| MI07 | 5 | 63667004 | CACTATCCGAGCATCTGAGTGTTTGCGAGATTGTG[C/T]GATTATGTGGTGCTAATAATA |
| 90 | | | TAGGTGTTGATGCC |
| MI07 | 5 | 64134193 | TTGACTTGTGATAACCTGGTGGTGATAGGAAAGTT[A/G]GGAGCAGCATGGGTACTGT |
| 91 | | | TGCTGGTATGGCAAGC |
| MI07 | 5 | 65992707 | TGGATGAAGTCGTCTAATCACTAGAACATCATGCT[A/C]AGTTGTTTTGAATATTGAAT |
| 92 | | | ATAAATTGTACATAT |
| MI07 | 5 | 66860524 | CAAATTTTTCAATCATTTTGCTTCCAGATTGAAAC[C/T]CCGAAGGATGTCCAACAGAA |
| 93 | | | GTTTGGGCGTACTGT |
| MI07 | 5 | 67852870 | AAAATTAAGTTTTGGTGCAGCTGCAGGTCAGTAAT[A/G]AGTCCTGGGATGTAAAGGC |
| 94 | | | AACTAAAATAAACTAA |
| MI07 | 5 | 69481320 | CTCTACTACATGGCAACAGTGATGTGGCTGACCCT[A/G]AAAATCATGCTGTTTTTTAA |
| 95 | | | TCATATCCTAGCCAA |
| MI07 | 5 | 70605990 | TGAGTGGTTCATGTATTACCAACCTAGGGAGGGCT[C/G]ATGCATAATCATGAATGAAA |
| 96 | | | TGCATGAGTTACCCA |
| MI07 | 5 | 71106594 | TCACTATTAATTTAAATGGTTTTCTTCATCCATAT[G/T]TTCTTGTATCTTATTCCTAGG |
| 97 | | | TGAGAATCATTAAC |
| MI07 | 5 | 71749089 | GTCTAGTTCAAAGACAGAAACAGCAGCATCACATG[A/G]CCAAGTTCTTATGAGAAGT |
| 98 | | | ACCAAAAATAAGCAGG |
| MI07 | 5 | 72568472 | GATGACCTAAGATGAGGTACTCGATATGCTACATT[A/G]AACTGTGTTCGATATGGTGG |
| 99 | | | TGAAGCTTGCCTCAG |
| MI08 | 5 | 73084362 | TCATTGTAACCGTGTGCTGACGAAGTGACGAACAC[A/C]CTGCTCACTGCAATTGCTG |
| 00 | | | CAGGAGGCTCTTTCAG |
| MI08 | 5 | 76250654 | CCACATGTGATCCCCATCAACGCATGCTCGCTCGG[C/T]CGCTCCCTTTCAAATTGCAA |
| 01 | | | GCCAAGCCGTCTCAA |
| MI08 | 5 | 77469219 | CACTTTGAAATGGCTCTTTCCGCGGCAAAGTACGA[C/T]GCAAAACTAGCAGTTGCGG |
| 02 | | | GCAAGGAAAAGGGATC |
| MI08 | 5 | 80224009 | ACCTCTTGTTAATTTTTTTCTAAAATATCACTACA[A/G]TGAAGCATTCCATAAAGAATT |
| 03 | | | TATAGGGTCCGTAG |
| MI08 | 5 | 81802084 | CTTGCCTTTGGAATCCCTCTAGACTGGGTTGCTGG[C/T]TGGCCCCAATACGGGTTGG |
| 04 | | | GCCTTGTCACGAACCA |
| MI08 | 5 | 82946410 | TTATTAAGCTTTGGGCCAATATATCACAATCCAAA[C/T]AGACCAGGATATCACAATCC |
| 05 | | | AAACAGACCAGGATA |
| MI08 | 5 | 84459929 | TATAAATACACCCAGTTACCATGTCGCTCTCCATA[A/C]CCTCAACACCCTGAGCCAAG |
| 06 | | | GCAAATCCCCACCCA |
| MI08 | 5 | 91893668 | ATACGCTCTCTCTATTCCAAAACAAGGCATTTGAG[C/T]TTTTTTAGGTACATAGTTTTT |
| 07 | | | CTATACATCTAGGG |
| MI08 | 5 | 92335501 | TAAATTCCAACATCTAAATACCTCCAAACAGTCCA[C/T]AAGATACCCCCTGCATTCTG |
| 08 | | | TTTTTGGACAGCAAC |
| MI08 | 5 | 93268903 | GAGATTGTGAGCAAGTGGACTGGTATTCCAGTGTC[A/G]AACCTTCAACAGTCTGAA |
| 09 | | | AGGGAAAAGCTGCTGCT |
| MI08 | 5 | 95753236 | GACATTACGCTTTTCAAACTGATTTCTACCCACTG[A/G]GAAAGGTTCTATGGCCCCCG |
| 10 | | | ACGGAGAGATTCTCA |
| MI08 | 5 | 95794884 | ATTTAAAATGATAAACACGACACAGAGGGAGCTTC[A/G]TCAAGATAGTCCAAGCAAT |
| 11 | | | GGCGAGGACTGGTATG |
| MI08 | 5 | 10163721 | GTTTATACAATTCTAACTAACAGTGTCGCAGCTCT[A/G]ACAATAATCAATGTAGTTGC |
| 12 | | 2 | TGCCCTCAGTTCTCG |
| MI08 | 5 | 10337171 | AGGCTCCTTCATCAACGTCTGCCGTGTGATGTCGC[C/T]AAAGCCTCTGCACCATCAC |
| 13 | | 5 | CGGAGAAAGCCCTCAG |
| MI08 | 5 | 10468973 | TCACCTGCGATCGCTCATTTGTAAAGAACAATTCG[A/G]TTATTAAGCTGGAGTATTGT |
| 14 | | 5 | TTTTCTCGGACAACA |
| MI08 | 5 | 11518649 | ATGGCCCATAATTTATTACTCAAAACCTAATCCAA[C/T]CCGACATGTCCAACAGTTAA |
| 15 | | 8 | GCACGAGCCAACCTA |
| MI08 | 5 | 12959523 | TATGAGCTGTAAAAAGCTGAGAGCTGTGGCGGTGG[A/G]AGTCACAGACGAGCGGAC |
| 16 | | 4 | GCCGCAATTGCCGATTG |
| MI08 | 5 | 13507400 | TGGACTTAAGTTTTTTTATGGTTCCGCTGTTTCCT[C/T]TCGATCTAGCTCTTCGCCTAC |
| 17 | | 5 | CTTTTTCTTGGCTT |
| MI08 | 5 | 13638979 | ATAGGGCCTGAATGTAGTCCTTTTAGAGTTTTGAC[C/T]TTCGTCTCTGCCACAATGGA |
| 18 | | 9 | GCCCTTCTCTTGATA |
| MI08 | 5 | 13696686 | TTCTCGATAGTCGGCCTATGTCCAATTACGTAGTC[C/T]GAATCCGCATTCTCCAGCGG |
| 19 | | 0 | AATCTCTGTACTGCC |
| MI08 | 5 | 13717707 | GGTTCTAGAACTCTTTGATGTTATGAAACGTTACT[A/C]TATTCTCATGATATCGATCTT |
| 20 | | 5 | ATGGATTAACTATT |
| MI08 | 5 | 13737696 | TTGCATGTTCGCTGGAATTTTTCCTAAGGACGGTC[C/T]GCCCCCAAACTCAGCCCAG |
| 21 | | 9 | ACGGTCCGCAATACAA |
| MI08 | 5 | 13981057 | AAATCATTTCTCGAAAAAAAAATTGTTTGCTGATA[A/G]AGCTTCTTCTAAATTCAGCG |
| 22 | | 5 | CGAAAGCTTCTAATG |
| MI08 | 5 | 14218015 | GAAATTGCTTCCCATGGTGTCGTTGTTTGGACTAG[C/T]GGTTCTAACCAACTAGTGAA |
| 23 | | 2 | ATGATGTCGCGTGCT |
| MI08 | 5 | 14320207 | GGGAAAAGGAACATTTCTCCTGTCTAATCAACGTG[C/G]TCGAGCGAAGAGATAAGAT |
| 24 | | 9 | CTTTCACATTGAAAAT |
| MI08 | 5 | 14629912 | ATAAGATTTGATGGTTACTTCGGAGTCAGATGAAC[C/T]TGTACTCGTTATTTATATTTG |
| 25 | | 1 | AAGATTTATATCGA |
| MI08 | 5 | 14811223 | GCTCCCGCGCAACCACCAACATAGCTGAGGGAGAT[A/C]GCGTGAGCGAGGCAGGGG |
| 26 | | 7 | CATCGGTGTTCGTCTAG |
| MI08 | 5 | 14953332 | CTTCCCTCGGCCGTCGAAGGCTGCAGACAAAGCAT[C/T]ATCTTTGGCCTTGCTATATG |
| 27 | | 9 | CCCCGAGTAGTTCGT |
| MI08 | 5 | 15146122 | TCCCTAGTTGTGTGTCTTTAAAAAAGGATTTTTTT[C/T]CTGTTTGAGCTTAAGCCTCT |
| 28 | | 8 | CTGATAAGGCATACA |
| MI08 | 5 | 15215381 | TATTGATCTTTAGTAATAGGTTGTGATGAAGTAGC[A/C]TACATCTCCTTTGGAACCCA |
| 29 | | 0 | AAATTTTGTGGTGTT |
| MI08 | 5 | 15215941 | CAAGGTGGAGATATAGGGGAGGAGATTTTGTTGTC[A/G]TGTCGAGATAGTGGGGCAT |
| 30 | | 7 | AGGGGAAGAAATATAG |
| MI08 | 5 | 15479721 | TTGGCGCATGCAGTGGAAACTAATCCCAAGATCTA[C/T]AGACACAACTATTTAAAGT |
| 31 | | 7 | GCGCACTGATCGAGTG |
| MI08 | 5 | 15509389 | CTTGTTGATGTGTGGAACATAGGAAAAAATGAGGC[A/C]AAAAGTGGATTTATATAAC |
| 32 | | 0 | AACAACCTACAAAGTA |
| MI08 | 5 | 15648538 | TAGGATTGGAAACAAAAGTCTAGCTGCCACAGCAG[C/G]GATGTCGTTCGGCCTGCA |
| 33 | | 1 | GAACCCACTCAACGAAG |
| MI08 | 5 | 15915314 | TTCTGAATTGTTTCAGTACACGTACAAGTGTGTTT[G/T]TATAACTTGTTATTCAGTTTG |
| 34 | | 4 | TGTAGTGTGCTCAC |
| MI08 | 5 | 15937144 | TCCGGTAGCCGTCGCCAGGCTGCTGTGAGTGTGAC[C/T]GACCTTCGACCTCGATCGC |
| 35 | | 0 | TAAGTGGTTGCGGTAT |
| MI08 | 5 | 16281696 | GGCTCTAACGAGGACTTAGGAGAAGCCTAAGATTC[A/C]TGATACCTCGATAAAATAC |
| 36 | | 9 | CAAAGTCATTGACGAG |
| MI08 | 5 | 16316023 | CGTCATTGTCCTGCTAATTTCGTAGCGTTCATTGT[C/T]TTTGTATTCATATGTTAGCAC |
| 37 | | 9 | ACATGCTTCTTCAG |
| MI08 | 5 | 16469579 | TTGGTCGGCTACTCTGGTCCTCTGGTATCCAAGCG[C/T]CAGACTCAGATCCCTGCAG |
| 38 | | 0 | CCACAGCCCGTGTGTC |
| MI08 | 5 | 16728180 | GGCAGTAATGTACTGGTTTCCATCTAGCTACGTTA[G/T]GCATTTACTGCTCAAGTCATT |
| 39 | | 9 | CATGCTTTTACTCA |
| MI08 | 5 | 16746753 | GCAATGCAAATCGATGGTCCAAAGAATTTACTAGA[C/T]AAGAGATAGAATATATTAGA |
| 40 | | 3 | TTTCTTATGTTGTCC |
| MI08 | 5 | 16751165 | CTATATGCTGGCTGGAGGAATTTGTGATTGATGCC[A/C]ATCTGATCAAAGTTATGTTAG |
| 41 | | 8 | AAAGTTATGTCGGC |
| MI08 | 5 | 16887925 | TTTCTATGATCCACCTTTTGATAAATTTGACACAT[A/G]AAGATCAATGCATTACATAGA |
| 42 | | 6 | TAATTATGTGTCAC |
| MI08 | 5 | 16889323 | AGCACTTGCCTTGTGCACTGTAGTTAGTCTGTTGC[A/G]TCATAGCCGCATCGACCATA |
| 43 | | 6 | GCTATACATGTATCG |
| MI08 | 5 | 16928023 | TTTAGAGCTCCAGTTCATGACTGCATCAATGGTTC[A/C]TCGTTAAACTTTGATTTATG |
| 44 | | 0 | GGTAGCATTGGTCTA |
| MI08 | 5 | 16995685 | AGGTGTCAGAACATGCGAGGAAACCTAAGGGTCAG[C/T]ACGTTGTTGTAGTGGATG |
| 45 | | 6 | GCTCTCCTCCATCCTAG |
| MI08 | 5 | 17192219 | TCGACTGACTTGGGATCCATAGTCACCTTCTATGA[A/G]ACTCTCTGAACCCTCAACCT |
| 46 | | 2 | AAAAGGAGAAAAAAA |
| MI08 | 5 | 17278585 | TGAGAAATGGACTATCTTCGCCTGCAGAGGCATGT[C/T]ATAAAAAAGTGGCTTACAT |
| 47 | | 2 | GCTGGTCCATTTTTAA |
| MI08 | 5 | 17384621 | GCTGCCAATTTGGCGAACAAGCCAGCCGCAGAGAC[A/G]TTTGCACGCGCCACTTCA |
| 48 | | 4 | CTCAAAGATCCATTTAT |
| MI08 | 5 | 17411663 | GATCGTCCTCATTATCTGCCACCTGATTAGCCTCG[C/T]CATCTGCCGTCTGATCTACAA |
| 49 | | 5 | CTGATTGATCTAAC |
| MI08 | 5 | 17419522 | ATCCTCTTGTTCCTCCCTCCGAAACCCTGGCAAAA[A/G]GAGGGAAGGAAAAAAAAA |
| 50 | | 1 | TGTTTCCAAATGCGATT |
| MI08 | 5 | 17445671 | TGAACACATCAGATTCCATATTTTTTTGTTTTGTC[C/T]CGTTGACATAGTGCTTTCTTT |
| 51 | | 5 | CTTATGCCAGAGAA |
| MI08 | 5 | 17575164 | CCATGACATGTATATGCGGGCCGTAGCCCTAAAGT[C/T]CTGGACGGCCCATAAATCCC |
| 52 | | 8 | AGGTCTTGCCCCCAG |
| MI08 | 5 | 17608256 | ATGCATGCACGCCATTAATTCGTGGCTTCTGCCTT[A/C]TATTTATTCTTCCCATGGGCA |
| 53 | | 7 | TGGCCTCCAGAGAG |
| MI08 | 5 | 17608275 | CCCTCTTGTACGTACACGGCAGTTTTAAGTGAGTT[C/T]TCTTATCCCTGTGCTATCTGT |
| 54 | | 2 | AAATACATACTTTG |
| MI08 | 5 | 17615879 | AGCATTATTTCTCTAACGCATTGTTGGAGAGAAGA[A/C]AGGACGGAACTTCGATAAT |
| 55 | | 7 | CGGCAAACTTGACACA |
| MI08 | 5 | 17802428 | AACTAACCCCTAGTGCTAGCGCACGGTGTACGAGA[A/C]CTTGGGGTGCTTTCTGCAT |
| 56 | | 1 | GAGCGTGGACTATCTG |
| MI08 | 5 | 18199328 | GACATTTAGCCTTGATGGTGGCTATGTTTTTTTTT[G/T]GTTTATGCATTACAACAAGTT |
| 57 | | 5 | GAAAATTGATGATG |
| MI08 | 5 | 18208990 | TAAATAAAAAGGAATGAAATGGCAGCGCAATGCAA[C/T]GACCACCAGTTATTCGAAT |
| 58 | | 7 | AAAGACTGCGTTTGGC |
| MI08 | 5 | 18258887 | GTAACATCTTTGCCTGGAATGATATAACAAAAAAC[G/T]TATAAATGTGTGACCTAGAA |
| 59 | | 8 | ATTGTAAAGAATTAT |
| MI08 | 5 | 18411425 | CTTATATTAATTGGTGGTTCCAACCAATACTAATA[C/T]CCCTTCTTAGAGCATCTCCAA |
| 60 | | 2 | GAGCTCTCCATAAA |
| MI08 | 5 | 18428274 | TGCTAGGCTGGACAGATCTTCCACACCAATAGCTA[A/C]TAATCATTGGAAAAAGTTG |
| 61 | | 5 | CAACAGATCTGCAATC |
| MI08 | 5 | 18454278 | CTGCTTTTGTGAACTTTGTCTAGCTGTATGTAGAC[A/G]GTTTATAGTTGTGTTGTGGC |
| 62 | | 5 | CTGAACAGGTTGTTT |
| MI08 | 5 | 18725134 | CCCACTTTCCACCGCAGTCACTGCACGAACGCACA[A/G]GACCAAAACAATTTGCAT |
| 63 | | 9 | CAAGAACTGAACTAAGT |
| MI08 | 5 | 18925120 | GCTTGCGCCTGGTGTGGTCTGTGGCTAGCTTGCTG[C/T]CCCACCGGCACAGCGTCGT |
| 64 | | 9 | ACAGACGATACAGCTA |
| MI08 | 5 | 18925303 | AGCGACACGTAATGCAAATAGCAACATAAACACAC[A/G]ATCTCATTTGATATTTTTTA |
| 65 | | 9 | TGTTAAGAGGCTAGG |
| MI08 | 5 | 18996203 | CAACTTCTCTATCAATGATGCTACTACTTGGTGGG[A/G]CTCGCTATAGTTAGCGACTAT |
| 66 | | 4 | GACTTCCCTCTTCT |
| MI08 | 5 | 19026233 | TACTAATAATTTCCATAATGAACAAGGTAAAACAT[A/G]GATGGGTGCAATTGAGCAG |
| 67 | | 3 | ATCACAAATCCAGCAG |
| MI08 | 5 | 19117217 | ACTTGCGTTTGTAAGGTGATAGATGCCTAGAAATT[A/G]GAAATGAAAGCAACTGTAG |
| 68 | | 2 | CTATTAGCTAGGTAAC |
| MI08 | 5 | 19118305 | AGCTAGGTAGCTAGCGACGACCTAGCGTATGTAGG[G/T]AGGGAGGGTCGCGGCTGG |
| 69 | | 7 | AGCTGGATTGCGAGGCG |
| MI08 | 5 | 19226106 | AGCATAATGCAAGGTCCCTTGGACGATGGAGGCGA[C/T]GACGTTAACATTGTCTCTA |
| 70 | | 8 | ACGCTCGCGATATATA |
| MI08 | 5 | 19248240 | AGGGAGACCCTGATTGGCACGGACGGCCACCGCCG[C/T]AACCAATCATTCCTCGGC |
| 71 | | 1 | CTCCCAAACCTTCATCC |
| MI08 | 5 | 19378636 | AATGGAAAGAAATGAGGTAGTGGGACCACCATAGA[G/T]GCATCAACCGACCATGCTT |
| 72 | | 0 | ATGGATCCACCAGCCT |
| MI08 | 5 | 19487615 | GGATGGCTTAACATATGATAATAGTACCATGTGGA[C/T]AGCTGACTGCAGGTGCAAA |
| 73 | | 1 | AGCATGGTGTTTATTG |
| MI08 | 5 | 19554183 | GGTACGTCCGTTAAGAACCCTCGAGCACCTACAAT[C/T]GACCCAATCAATCTACATAT |
| 74 | | 1 | CATTTCCTTTACTTT |
| MI08 | 5 | 19559687 | TATACGACACGGATTGCTGACTGCAACTTGCACCG[C/T]TAAACAAGGCTAGCTTGAC |
| 75 | | 4 | ATGGTAGTATCCGGCA |
| MI08 | 5 | 19716833 | TCGTTTCTAAGGAAACTCAGAAGCACAAGTGGCTC[A/G]AATTAATGATCCTCATGTC |
| 76 | | 4 | TCACCAAATCCTTCAC |
| MI08 | 5 | 19836790 | TGGCTATAGGTGTCGGACCGATTCTCTTTCCCGCT[C/T]CTTCATGCCAGTTGTGGTCA |
| 77 | | 2 | TGCAGCCTGTACTTG |
| MI08 | 5 | 20247553 | CATGAGTAAGATCGTAACCGACAAACCGTGACATT[A/C]CTGGCGGTTTCAACAAACA |
| 78 | | 1 | CAACAGGACAATTCAA |
| MI08 | 5 | 20317286 | GGGAGCTTCACGTTATGGGAAAGCAAAGGTAGAGT[A/G]GTTGGCTACATCTATGAAC |
| 79 | | 3 | AATCAATGTTAATCAT |
| MI08 | 5 | 20488668 | ATTCTGCTGTAACTTATGTCACGGGTCGTCCTCTC[A/G]GATATGCCGGATAAAAGGCT |
| 80 | | 4 | TATGTGCGCCACCAA |
| MI08 | 5 | 20496060 | ACGGTGCTATGATAGAACAGGTAGGATTATCCCCG[C/T]GTCTACTTTGATGATACCGC |
| 81 | | 8 | GTGCCAAATCACGGA |
| MI08 | 5 | 20520730 | ACTGTACCTAGCAGTACTTTTACGGCACCCAAAAT[C/T]TGCGCAAACCCGATTCGCG |
| 82 | | 3 | TGTGTGGACAGTTTTC |
| MI08 | 5 | 20609050 | TCTAGTGGAATTGGAATACAGCACCAGGCAGCAGC[C/T]GAACTCAACTTGGAGCCG |
| 83 | | 8 | ATTGCATAGATTAGCAA |
| MI08 | 5 | 20778095 | TAATATAACTGAAGATAGTTCAGTTAAGAAGTTTG[A/C]CTGCTCATAGCAGACATTAA |
| 84 | | 6 | GGAAAATACCTGTTC |
| MI08 | 5 | 20840089 | AGAATTGATGGGTGCAGTGCAGAGTGCAGACGATA[C/T]GAGTGTGAGCACACAATT |
| 85 | | 3 | AAGTTTGCATGGGAGAA |
| MI08 | 5 | 20860080 | ACAAAGCATAGGTACTACACAATCGACCTACACGC[C/T]GAATGAGCCCACTAGCCCA |
| 86 | | 5 | TATATGCCTCCACAAT |
| MI08 | 5 | 20879969 | AAACCTAAACACACCGCAAAAAAGGTATCAAGCAT[A/C]AAAAAGGCAAGGTGGGT |
| 87 | | 5 | GAGCACATAGGAAGACAA |
| MI08 | 5 | 20884922 | GTGGTCATGCCTGTCTGCTTTCAGGACGGCATGCG[C/T]AGGCTATTGTCGCGATCTTG |
| 88 | | 6 | GGTTCGATTTGGTGC |
| MI08 | 5 | 20893865 | TCTGCGCTAGACCGAGTCCGAAAGCTCCCTCCGCT[A/C]CGCAGCTGGTCGGTCTAAA |
| 89 | | 3 | CTTGCCGTCTCCGTTT |
| MI08 | 5 | 21158765 | CTCTACTATGTAAACAATGTAGTTTATAGTACAAA[A/G]GCAGCAAAACAATACTTTAC |
| 90 | | 1 | ACAAACTTCTAGAGA |
| MI08 | 5 | 21222792 | AAACCACCTTATGTGAAAACATTTTACACCAAAAA[A/C]AATACTCTACATCAGATTCT |
| 91 | | 3 | TTTACCAATGATATA |
| MI08 | 5 | 21334091 | CGGCTGCAATCAACTCTTCAGCAGCATCTTTTACC[A/G]CTGCGCCTTCTTCTGTACCT |
| 92 | | 1 | TCAGGAGCTTTGGTT |
| MI08 | 5 | 21358676 | ACAGGCCACTACGTCCCCCAGTTGGCAAGGAAGAT[C/T]GTCGAGTACAACGAGGCC |
| 93 | | 1 | TCGCCTCACCCCTTCAT |
| MI08 | 5 | 21630149 | GCTACTGTGCCATGGTAAAACCGAGTGTTTTACCT[A/G]CGTTGTCCGTATGCGTCTTG |
| 94 | | 3 | GTCTCCCAATCGTCT |
| MI08 | 5 | 21679601 | TAGAAATCCTCGGCACACGCTGGGTCGAACCAACA[A/G]CGGACATAATTGGACTTTT |
| 95 | | 6 | TTATACTACAAACTGA |
| MI08 | 5 | 21759580 | ATGTTGCTTTTGTTGAGTGACATAACTTGGATCTC[A/G]CTTTTGTTGAGTGGCATAAA |
| 96 | | 0 | ATCCAACCAGTGCTA |
| MI08 | 6 | 1537980 | ACAACCCATCCATCGATTCTTCAAAGCTACCAGAG[C/T]AATGTATACCCCCTCCCTCA |
| 97 | | | GAGGAACAAAAGTAT |
| MI08 | 6 | 4803062 | CAATCAATTTCAGGCTCCACTGTTGCCCAAGATGG[A/C]CGCTAGACCTTAAGTGTCG |
| 98 | | | CTTAGGTGCTAGGCAG |
| MI08 | 6 | 4846717 | ATCACTTCTTTGCTTCTGCAACAGGTCTTCTTCGG[A/G]CGGGATATCGTAGTGAAGGA |
| 99 | | | AGCCCATGTAGAGCG |
| MI09 | 6 | 5870567 | TAGGAAACAGAAATTAGAGGGCACTGAGTAAGATA[G/T]CAATATCCAACAGGCGTAA |
| 00 | | | GCTGATTACTTCTTGC |
| MI09 | 6 | 7161363 | AATCTCTAAAGAAGGTTAGTGTCTTTCTATCTGAC[A/C]TTTGGTGTCTTTCGTTCATAA |
| 01 | | | TTTCCCCCCTATTT |
| MI09 | 6 | 8281009 | ACGCACTATGCCCGGGGTTAGAGTTATAAATAATT[C/T]GGAGTGCGGAAGATAGTTCT |
| 02 | | | CCTTGCTAGGAGTTG |
| MI09 | 6 | 9190630 | CCTGCAGGCTCTAGGGCCATAGGAAAGGAAATGCC[C/T]CGAGCCCCTGCCAGACTC |
| 03 | | | CGATAAACCATCTCCTC |
| MI09 | 6 | 9914853 | TATGGTACCAATCCTTCGCTTTTTCTGCCCATCCA[C/T]GTTTTTTGCAGCCGAAGGCT |
| 04 | | | CCTCTTTCTTCTGTA |
| MI09 | 6 | 13019148 | AATAGACAGTATTTCCTATAAGGAAAATAGTGAGA[C/T]AGAGAGTGTGTGAAATCTG |
| 05 | | | CCAGGTTGGGCAGGGC |
| MI09 | 6 | 16802233 | GGCTCTCAGGCAAGAGGTTTATCTATACCTTTATG[C/T]AGCGATGGATCCAGCCTCTC |
| 06 | | | AAGGCCTGCCGGAAC |
| MI09 | 6 | 17956521 | CTTATAGACTACATGTACTTTCATACCATAAACAC[A/G]ATAAAAAACTAAAACATAAC |
| 07 | | | TATTGTACATGGTTT |
| MI09 | 6 | 23162906 | AGTTAGGCAGCAAGCGACCTTCAGCTTTACAAATT[A/G]GTACACATGTCCTTCAGCT |
| 08 | | | TGCATTCAGTGTGCAT |
| MI09 | 6 | 23829343 | TCAGATTACTCACTTTAAATATGATGAGAAAAAAT[A/G]GATATGAGAACCATGAGTAT |
| 09 | | | ATGAAGGATCGAATA |
| MI09 | 6 | 24313682 | GTGGGGAGATGGCTACATTAATTACTGTATAGTAG[C/G]AGCTATTCAAGGGACACGA |
| 10 | | | AAGGAGGGGGGAAATA |
| MI09 | 6 | 24552959 | TCAGTAAACATTTGGTTTGCATATGACTATGAGAG[A/T]TTGGTACTGTATTATCTCTGA |
| 11 | | | CAAAAGAATGGTAA |
| MI09 | 6 | 24601477 | ATTGGAGAAATTATATTAGGGTAGGATACAACACG[A/G]GATGACCTAGTTAGGAATAG |
| 12 | | | CCTAACCATAGTTAA |
| MI09 | 6 | 26228933 | GGGGCCCTCGCGTAACACCACGCCGCACTTCGTCT[A/G]TATCACTTACGTGTGGGAC |
| 13 | | | CCACCCGTCAGGGTTT |
| MI09 | 6 | 26667965 | CCCTTCCTTCTTTCCCGTTCCCGGGCTAAAAAATA[A/T]TTGCTAATCACAGCAGGGAC |
| 14 | | | TGAGGGACAAGCTCG |
| MI09 | 6 | 31309851 | CCTCCGCCCCCTTCTCTGTTTCGTAACCGGTGTTG[C/T]GATGCCCTCTGCTCAGTGAC |
| 15 | | | AAACTCCCCGCCCGT |
| MI09 | 6 | 37900870 | CCATCCTCACGGCGGCGCGGACCACGCCGGTGGCC[A/G]ACTCATGGTGAGCGTGAT |
| 16 | | | ACCTTCACTTCACTTTC |
| MI09 | 6 | 38769809 | TTAAAATGCAGGTACGTGAAAAGCATCAAGGTAAA[G/T]ATCGAGGGGTGACAAGGG |
| 17 | | | TTAAGGTGATAACAGAC |
| MI09 | 6 | 38780600 | TAGAAACATCATAAATGGAACCAACATCATCATCT[A/G]TAACCAAATCCATAAATAGA |
| 18 | | | ACCATAACATCTCGA |
| MI09 | 6 | 39077982 | AGAAATTTATTTACATGAAGATAGGTAACGTGAAG[G/T]AACGGAAAAGACCCCGCCT |
| 19 | | | TCTTTCCCTATTTGAC |
| MI09 | 6 | 41632602 | ATACAGAGCAATCAATTTTAGTTTAATGAAAGACA[A/C]AGCTGCATCTAGAAGAAAA |
| 20 | | | TAATACAGTGAGTAAA |
| MI09 | 6 | 48011626 | AGGCTGACCAATCCAGCAGGGGGCACATATCTACA[G/T]CCACCAAGATGACGACAA |
| 21 | | | AAGGGGACATCATCAAA |
| MI09 | 6 | 54018121 | GTTCCAATCCCAAAGGACTTTGCAGCTTGAATGGC[A/G]GAGAATCAGTCATTACCTT |
| 22 | | | CAAAATTTCAACCACT |
| MI09 | 6 | 55751842 | AACTTCATTCGAGAGTTTTAAGATGCATGATTGCG[A/G]CATCTTTTATTCTTGAATTTC |
| 23 | | | ATCTATATCTATCC |
| MI09 | 6 | 58605917 | ATGCCGATGAACCAGATAAGGCCACAGAAGATGGA[C/T]ACGACCCAGAGGCCAGTG |
| 24 | | | AGCTGGTCAGTGCGCCG |
| MI09 | 6 | 59285267 | TCTCCGAGAGAAACTGAAGGGAAGCCGGAAGAACA[A/G]CAATGCTAATCTTGTAGG |
| 25 | | | TCAGGCATCTCGCCTCC |
| MI09 | 6 | 61173229 | CCTATTATGGGGTATGGCTCCGATCCGATGCCTCG[A/G]GTTCAAACCTATCCGTCCGG |
| 26 | | | TGTCATCGGTAGATA |
| MI09 | 6 | 61456996 | CAGCCCCCCCCTAAAAGAATCTCGTCCCGAGATTC[A/G]GAGCGAAAGATATTCAAGA |
| 27 | | | GTAGAGAAGCATGTAA |
| MI09 | 6 | 61836439 | CATCATCCTTCGGTTCTGGAATTATCTCTTCACCC[A/G]TGAACCTTATCAAGTTGTCAT |
| 28 | | | CATTCTTTCCAAAA |
| MI09 | 6 | 62022407 | AACCATAATGGATCCTAAGCAACATGAGCTTGCAA[C/T]CACCCGATCAATGATCTACG |
| 29 | | | GTCAACGTAGATCCC |
| MI09 | 6 | 62347165 | CCCTTCACCGCGCCGTTATGGTCACGGGTATTACT[G/T]TTGATATGCTGTCCCTGCTCC |
| 30 | | | CCAGCAGCAGCAAC |
| MI09 | 6 | 63302866 | TAGGTTCGTCCATTAGCTTCACCAGGCAGAGTAGT[C/T]CACCCGTTACGTCATCTTGG |
| 31 | | | GGTCGACTTGGCTCG |
| MI09 | 6 | 63473038 | GCACACGAAATTTCTCGTTGGAGACAAACTCCGTC[A/G]TGGACCTACTCCGCACCGC |
| 32 | | | GGGCCGACCTCACCAT |
| MI09 | 6 | 65458137 | CCCTCCTGCTTGTTGCTCAGCAGTTGCTGTCTAGC[C/T]TGCAGTATGCATGTACTGTT |
| 33 | | | TCACATGCATACTAC |
| MI09 | 6 | 65659738 | AGGAAATTTTTATGGCTTTGATCTCGATGGCACTT[A/G]GGCAGGAGCTTATGGGATGC |
| 34 | | | AACTGACCAGGCATA |
| MI09 | 6 | 66803432 | GTAGTTTGTAGGTGTGCAATACAATGACGGTGGTC[A/G]ACGGTGAAGGTGAAGATCA |
| 35 | | | TGCGAGCTTGTGCAGA |
| MI09 | 6 | 68945381 | TGCTGTAAAAATATCAAGGCCAAAAGGAGTGCTTT[C/T]GCAGTTTGAAGCATAACTG |
| 36 | | | TATTCCCTGATCCCTA |
| MI09 | 6 | 71303851 | CCATTGAAAAACCATTTCTTCAATGTGGTGATCCT[C/T]GCTTGAGTGTCATCTAGATA |
| 37 | | | AGAATGTTAAGATCA |
| MI09 | 6 | 73696655 | CCGGTATTCGCCACGACCCAGGGCAGTACCTCGAC[A/G]AAGAACCGTGCACCATCG |
| 38 | | | TCGTCCGGTGTCCGCCG |
| MI09 | 6 | 75292286 | GTGGAGGCCTGTGAAGGTATTTCCAATATGAATGC[A/T]GTTGTTTAAGGGTTATAGAA |
| 39 | | | TGTAGATATAACTTA |
| MI09 | 6 | 75557018 | CGGCTAGGATCACAAATGTTTTACGTGACAGAATA[A/C]CAGTATCTAGCCATTTTAGC |
| 40 | | | CTCATGTCGTGGTCT |
| MI09 | 6 | 76848126 | CCCAAACAGAACATCTATCTTTGAGCTATGTTTTC[C/T]AGTTCGAACCATCTTCCACC |
| 41 | | | ACTGTACAACGAAGT |
| MI09 | 6 | 76869501 | TGGGAAACGACGACGCCTCAAGGCCTAGGATGCAA[C/T]AGAGGAGCAAGCTTCCCA |
| 42 | | | CACCACCATAGCGCACT |
| MI09 | 6 | 80166847 | CGAGAAAACATCGTGTCAATGGTCTCTCTAGTCAA[C/T]TAGACAACGTTCTCGTACT |
| 43 | | | CTCGCAGGTACATCTC |
| MI09 | 6 | 80444365 | GCAACAGCGTCTTCTTCATCGGTCCGCCGCAAATC[C/T]AGCCGAATTCGATCTTTCCC |
| 44 | | | AATGGTGAATCATAA |
| MI09 | 6 | 80673195 | GACTATGAATGTTAACGAAGACAAAGGTGTGCCAG[A/C]TATAGCTTCGGCTCCACAT |
| 45 | | | ATCTCGATTAAAGAGT |
| MI09 | 6 | 81837501 | AATGAACAGTCAAACATGGAAGTTGTGGATGACCG[A/G]CAAACAAGGGGATGCGAG |
| 46 | | | TGCATATTGAATAAAAA |
| MI09 | 6 | 82183292 | CAGGATGTTTTTTGAGGAGGCAGAGAGAGGGGTAA[A/C]TGAGCTCTCACAGGCTAG |
| 47 | | | CAGATGGCCAGTAAGTC |
| MI09 | 6 | 83130638 | CTCCTGATCGAGAAGGAGACCGTGGACGGGGAGGA[A/G]TTCATGAGCTTGTTCATT |
| 48 | | | GACGGCCAGGCCGAGCT |
| MI09 | 6 | 83386135 | CTCCTTCGAAAAGAAATGAAATGATGCCTCCCAGC[A/C]GAAGAAGAAAAACATCTC |
| 49 | | | ACGCCACGCCTGCTCCA |
| MI09 | 6 | 84102096 | ACTTTCTGGCTGTGCATTTCCAAGGGCTGGTTGAT[A/G]TGTGATCTTGTAGTCTCTGT |
| 50 | | | GTTGTCGTGCGCTTG |
| MI09 | 6 | 86279943 | AAGGAAATATTTCAGTGGCTTGCAATTGCAATGAA[C/T]TTTGTTGATAAAATGTTGCT |
| 51 | | | TTAATATTATATTTG |
| MI09 | 6 | 86618275 | CTTGGACAATCATGATAAAGCGAAAGGGATCAATA[C/T]CAGATGTGCAACAGGTGTT |
| 52 | | | TTCTAAGTCTGAGCAT |
| MI09 | 6 | 88752794 | GTATGCTGCACGAGCACGAGTAAATACTACAGGCA[A/G]CAAGCAACTAGACAGACT |
| 53 | | | TAATCGCATCTCAGCCA |
| MI09 | 6 | 88861513 | GATTGTCAGACATGCAGATCGGTTAGAAACTTAGA[A/G]TGTCAAACCATTGGCATGC |
| 54 | | | TGCAGTTGCTGGATGG |
| MI09 | 6 | 89567007 | CCATTGTCCCAAGTCTTCCATCTGGCTCCAATTAG[A/C]CAGCGCCAAAGCTGGTGCC |
| 55 | | | TCAATCCATGAAGCCA |
| MI09 | 6 | 91404838 | AAAAGGATAACAAAATATTGTATAGTGCAAATTGT[G/T]TTAAGCGCACACTGGGAAG |
| 56 | | | CCACTGAAATAATAAA |
| MI09 | 6 | 93556797 | GGTAGGTATGGAGCACATACGTATATACACGCCCA[C/T]GGTCCGACGGTTTCTAGCAG |
| 57 | | | AAAAACCTTAGAATT |
| MI09 | 6 | 94911456 | ATGGAAATTGCCCTGTCTCGGTGACTCAAAATCCG[A/C]TTTAGTCGTCACCGATATAT |
| 58 | | | ACTAATTTGAGGGTG |
| MI09 | 6 | 95480976 | TGGAATTTTGGGCAACGGTTTCGTTTTTATCAATA[A/C]ACATTGATTTAACCGCGGTC |
| 59 | | | CATTTCTGGCCCATC |
| MI09 | 6 | 96335930 | CCAAGCTAGTGAGCAACTATACCCGACCATGACAT[C/T]GGAGGCTAGGCTAGCAAGC |
| 60 | | | CCATCCATGTTGGTCA |
| MI09 | 6 | 97779031 | TCACGTTTTGTTTCCGTCCGTCACCTGTAAATGAC[A/G]TTTGACATGTACTATAGGGT |
| 61 | | | TCGGAGTGTACCTGG |
| MI09 | 6 | 97872003 | ATGAATCTGCTATCTAAAAGATGATGTTCTAGATC[C/T]TGGATTTCACATTTTGACTGT |
| 62 | | | TAATGAAGGCTCAA |
| MI09 | 6 | 10030681 | CAGGTGTACAAGCCACAACCACAAGTCGTCGTCGT[C/T]CAGGCTCGACTCGAGGTC |
| 63 | | 7 | GAGGGAGAAGCAACTCC |
| MI09 | 6 | 10036647 | TTATTATGATATTTATAATACTGGACCTAAACAAA[A/C]CTCAGGAAGTACTAAATCAAG |
| 64 | | 4 | TCAAAACTCTCTAA |
| MI09 | 6 | 10275796 | AATCTTGTGTTGTCAATCCATATAAAATTTGAAAA[C/T]TGGTGGCCCAAAATTGAAAA |
| 65 | | 0 | AAAAGATTGACTTAG |
| MI09 | 6 | 10310041 | TATCTGTTGGGTGGATAAGATCAGGAACGAGCTAG[A/G]TGTAATCCTGCGGTTAGAA |
| 66 | | 9 | TCATTGTCTCCTATCT |
| MI09 | 6 | 10313338 | GGCCAAGTTGGAATCTTTTTTGCTGCCGTATAGAA[A/G]CGGGAGAGCGACGCGGATG |
| 67 | | 9 | GTTGTGGGAATGTGGG |
| MI09 | 6 | 10551017 | ACGTACGGCACAAGATTATTAGCTCACCAGCAGCA[A/G]GCTGAGCACGGCAAAGGA |
| 68 | | 0 | TTAGGTCAAAGGAAAAG |
| MI09 | 6 | 10560117 | GAAGGCAATCTCTTTCGCTCGCTTTGCGATGGTGA[G/T]GGCGCTCTCCTAGAGGAAA |
| 69 | | 5 | TTAACAAAACTGTATC |
| MI09 | 6 | 10705530 | TGCAAGCTGGCTACTAATACTCTACACAAATGTAC[A/G]TAACTATCTATCTGTTTTTAC |
| 70 | | 7 | TCTCACCTAAACCA |
| MI09 | 6 | 10718187 | GAAAGTGGGCTGGTCTAGGCAGCACTGTACAGTCT[A/C]GTGCCACCCACCGGACAG |
| 71 | | 5 | TCCCGGTGTGCACCGAA |
| MI09 | 6 | 10739028 | TGTGCAAGGTAATCCAAGAGAGACCATACATAGTT[G/T]GGAGGAAAAAAAACAAAT |
| 72 | | 7 | AAAAAGCACACGAAAAT |
| MI09 | 6 | 10792673 | GGAGCCCGTGACCTCGACCTCGACCTTGATGTACG[A/C]CGCAGTTTCTTGTTGGATT |
| 73 | | 7 | GCTGGCCATCTATCTC |
| MI09 | 6 | 10823190 | TTCCTTGATTATTACACAAACATTAGTAGTATGTG[C/T]CACTTCTTCGAGAGTTATGTT |
| 74 | | 5 | AAGTGAAGTTCGGT |
| MI09 | 6 | 11007437 | CGAGTTCATGCGCCTGAACGTTGTTGATCTCGAAC[G/T]ACCCTTCGTCTTGTATGAAC |
| 75 | | 1 | TCCATCACCTCCCTG |
| MI09 | 6 | 11119645 | ATTCTGCAATTTTTCTGTACATGCCATTTTGATCA[A/G]CTTGGCTAACGTCTGAAACA |
| 76 | | 4 | AAAACTCACCAAAGC |
| MI09 | 6 | 11132181 | TCATGGTCTCAGTCGGCGCTATGATCACCTGAAGG[C/T]CCTCAAGTGCTTCATGCCCT |
| 77 | | 3 | TCCTGTCCTTCCACA |
| MI09 | 6 | 11137409 | ATTTGGTTGGGGCTTATCGACGTGTATCAATTCAC[A/G]GCGCAAACTGATCACGTCCC |
| 78 | | 5 | TTCCGGTTTGGTTGC |
| MI09 | 6 | 11224881 | GCTAAGCATCATTCCTTAAGCATACTTAGGAACGA[C/T]TCTTTGGTCTAAAGGTAGCC |
| 79 | | 9 | CAGACATCTGGGACT |
| MI09 | 6 | 11302046 | CCGTTAGCGAACTTGGTTGCTATGCTGAGGAGATC[G/T]GCGGTGGTCTTCGTTCGCC |
| 80 | | 0 | CTCGCCAAAGCTCATG |
| MI09 | 6 | 11352741 | ATGACTTTTGGCTGGCAGGAGTCAGCAACTGAAAC[A/C]AAAAAAGTTCTCAAATTG |
| 81 | | 4 | ACGAAATTGGATTTTCT |
| MI09 | 6 | 11405045 | AAAAAAATCAATGGTGGTTTACGTTTTTTAAGAAG[A/G]AAAAGCGGAAACTTCCAC |
| 82 | | 0 | GACCACCCGCGCTCTCT |
| MI09 | 6 | 11679537 | GACAATGACTCGACACAGTGGATTCAAATCCGATC[A/G]GCGATTCTGTTACCCTAGC |
| 83 | | 5 | CCCAGTCCCGTCGTCG |
| MI09 | 6 | 11776891 | TTTTGTTCGCAAAGACGAAGAACGACGAATTCAAC[A/G]CATGCGACTATGCGAGTGT |
| 84 | | 6 | CGCCAACCGTGAGAAT |
| MI09 | 6 | 11868550 | TAGAATAGGTTAGGTTAAATCTAGTTGATAAATAA[A/C]CTTAGAGGGAAATAACCACT |
| 85 | | 7 | CCATCGCATTTGTAG |
| MI09 | 6 | 11886573 | TGGCCGTGGGTGAGCCATTTTCCCACAACAAAGAC[A/G]AAATAATATTTGTGAGGAT |
| 86 | | 5 | TTCTTTAAAAATAAAC |
| MI09 | 6 | 11917342 | TTCTCATAGTCCCTTACAGGGTCAGCATGGAAGCA[A/G]CTAGAGAATCAGTTTCACA |
| 87 | | 9 | CCCAATATCATTCTAA |
| MI09 | 6 | 11958204 | AACGTAAGTGTCTTAAACATTATTTCATCAAACCA[C/T]AGCGGATCTCATGGGTAATC |
| 88 | | 1 | TCCCACAGGAAGATG |
| MI09 | 6 | 12121375 | AGCGTCTTCATCATCGGCTTCTTTCTCTGCAACTA[A/G]CATAAGCATCTCCAGTCGCT |
| 89 | | 0 | TGTCGAACAGAGCTG |
| MI09 | 6 | 12647669 | CATTCATTGCACTTGCATCGTTGTGTTGCCAAAAA[A/T]TTTTACTGCAGTTGAAGGTG |
| 90 | | 8 | TTTGCATCCTTCTCA |
| MI09 | 6 | 12763443 | CTAGTTTTCTTTAAAAAAATATCAGGAACACGATG[C/T]AGCCGATCGAGCTTGCACG |
| 91 | | 6 | CGCGCGTACGCCCATG |
| MI09 | 6 | 13054169 | GAGTGGCTCATGGCATACTCTGCATTGGTGTCTCC[C/T]AGGGGCAGGTTGAATGCCC |
| 92 | | 9 | TCACGCACGAAGGCTG |
| MI09 | 6 | 13154444 | AATATTACATTCCACGATTCCTGCTCCAGGTTGAC[C/T]CCTTGTCCCATCTTTTCATTC |
| 93 | | 4 | TCCTCTCTTTCTTT |
| MI09 | 6 | 13597936 | TCCTATTTTCATTTTTTAAAATAATTGTCACTAAA[C/T]GCATGAGTGTGTCAAGCCGTC |
| 94 | | 0 | AAGCGACAAAAACA |
| MI09 | 6 | 13784243 | ACACGCATACTACAGAAAACTAGAGTCCAAATGCA[C/T]GGCGCAAAAGTACACACG |
| 95 | | 7 | ATGAATACGTCATTTCC |
| MI09 | 6 | 13958389 | ACAGTAAAACCTCAAATTTAGATTATAGAGTAGTC[C/T]ACTAGGGTATAGTTATTTGA |
| 96 | | 4 | GACACTTGAGAGAGG |
| MI09 | 6 | 13987441 | AAGCACGGCCCTTGGCACATGCTTGCCGTTGACCC[A/G]CTGTTGCTGTTGCTGCTGC |
| 97 | | 5 | CGCCGCCGCCACTGCC |
| MI09 | 6 | 14018317 | CCAGCCCTTGGGTATGATCCTCAACCACCTTCGTC[A/G]CGTTTGTAGGAGTGTGTAG |
| 98 | | 2 | CACCTGTCCGTTGACC |
| MI09 | 6 | 14086372 | GCTGCACCTCATCGCTAACAATCAGCCGCAACGCA[G/T]GCACCAACCACGGATTCTG |
| 99 | | 6 | CTCAAGATTCGTCCTC |
| MI10 | 6 | 14116842 | CAATGAGTAGTTGGTTCAATATACAAGTGGCATGA[C/T]ACAAATACAGAGAGATAAG |
| 00 | | 1 | ATTAAGGGAAAATATA |
| MI10 | 6 | 14227489 | ACCAATGATTCAGCTGGTGATGGGACTACAACTGC[A/G]TCCGTCCTTGCTCGTGAAA |
| 01 | | 1 | TCATCAAGCTAGGCCT |
| MI10 | 6 | 14239024 | CTATAACGGGTGAAATGTAATGGAACGACTAAAAC[G/T]GTAGTCGAATGTCTGAAAC |
| 02 | | 9 | TATAACAGAATGGCTA |
| MI10 | 6 | 14250093 | TAAGGTTTAGGTGACGAGCCTAATTAAAGCATACT[A/G]AGAGGCTACTTCTACTACGT |
| 03 | | 8 | ACCTGCCGCGCCGGC |
| MI10 | 6 | 14253418 | GTGTTGCATAACCAGATTCCTCAATGCCCACAGTT[A/C]TTTGGTCCTCTCTCTCCACT |
| 04 | | 0 | CGGTGCTGATCTGAT |
| MI10 | 6 | 14349401 | ACTTATGCACGAGAATGACTACTAACTATTATGTC[A/C]TTGATTCTTGATTTTATTTGT |
| 05 | | 6 | ATTATATTTCTAGT |
| MI10 | 6 | 14394619 | CCTTTTTATTTTTGTACACACCTGGATGGATTCGG[G/T]GTCAGAGTACCATTAATCCAT |
| 06 | | 0 | TTATGGCAATAAAA |
| MI10 | 6 | 14666020 | AGATCAATTTATGGGTGTTGTCGAGGCTAAATTTA[G/T]GGCATATGACTATGAGCATG |
| 07 | | 0 | CATTGAATGAATTGT |
| MI10 | 6 | 14675572 | CGCTTGTCTATATAAAGCTGGAAGCTCTGCTCCTC[C/T]CGCCATCCCACATCCATCTC |
| 08 | | 8 | CACCTCCCTCATACA |
| MI10 | 6 | 14785372 | CAAAACCAATGCTCAAACTCCACCACACATACTCT[A/G]AAGGGCCTAGAATCATAAC |
| 09 | | 2 | CACAAAATTTTGGACT |
| MI10 | 6 | 14799941 | ACACGACGACACGAGCGACGTGGGCGTATTTATAA[A/G]GTGCGGTGAGACGAGAAG |
| 10 | | 9 | GATCAAGGTCAGCGCGG |
| MI10 | 6 | 14945490 | TAGACGTCGTTATAGCCGTCACCGGGTTTAAATAT[C/T]GACGGCCCCAATAGGTTGTT |
| 11 | | 1 | TGGCGGTGACCGTTG |
| MI10 | 6 | 14952880 | GGACACATAGATAAATCATTCAATCATTACATACA[C/T]ATGTGTACAATATGTATTAAG |
| 12 | | 4 | CCAAAAGTAGTTTG |
| MI10 | 6 | 14986477 | CCACAGTATTTGCTATACGTGCTTGTTCCCAGCAT[A/G]CCAGTATGTCTGATATTCAGG |
| 13 | | 1 | CCCTCTCCTTTCTA |
| MI10 | 6 | 15108063 | CTTTGGCTAACATTCTGCAGCAACCTGGGGGCCCG[A/G]CAAAACGCTTCCGTCCACG |
| 14 | | 5 | CGAAACTGATCGAAAT |
| MI10 | 6 | 15212457 | ACCAAAAGATAACGAGATCACCCATATATAATACT[A/T]TCTGTCTCAATTTATAATTCG |
| 15 | | 0 | TTTGACTTTTGTAT |
| MI10 | 6 | 15245673 | ATGTTGTCTAATTCAACTTTGAATAGGCGTTTTTA[G/T]ACTGAAGTTGCTTTTATTGCT |
| 16 | | 5 | TGTTGTTTTATTAA |
| MI10 | 6 | 15312832 | CTAGTCGACCTAAATAAATTTAGCCCAAAAGACTG[C/T]GCGATTCATTGTTGAAACG |
| 17 | | 9 | GAGCTTCGCCTTGCAT |
| MI10 | 6 | 15427768 | GCATGCAAACAAGAACATAGATTTACAGTGGCTTG[C/T]ATCATCAATTGAGCTATGTT |
| 18 | | 5 | TTTTCGATAACCAGT |
| MI10 | 6 | 15428221 | CTTGGCCTGCCTCTTTTTTTAGTAAACTACCACAC[A/G]ATCATGTTATTTTGTACAATT |
| 19 | | 4 | TCATAGCTAATTGA |
| MI10 | 6 | 15436918 | TGGATGGATGGATACCTCTGCAATAGAAGAGATTT[A/C]GCACAAATTACGAAACAAA |
| 20 | | 5 | TATGAGACGGAGCAAA |
| MI10 | 6 | 15694396 | TTTGCGTGTGGCACACTGGCACACCGTAATCCACA[C/T]CGATCATCGTGGACCGTGG |
| 21 | | 5 | TGAAAGCGCGCGTAGC |
| MI10 | 6 | 15856695 | CAAATACTTAATTTTGGGGATTGATGGAGGGGATT[A/G]AGGGAAAATTAACTAGCCC |
| 22 | | 1 | TTAACAGAAAATTGAT |
| MI10 | 6 | 16052401 | GGCGTCGTGGCCTCCCAGAAAGAAAACGCGTTTGC[A/C]GGATCGGATCTTGGCCGG |
| 23 | | 6 | ACGCGGCCGCGGCGGCC |
| MI10 | 6 | 16089726 | CTCCAAAGGAAGAGTTTCACGCTCCAGCTTTCGCA[A/G]GAAAGCCCTCAGAGAACC |
| 24 | | 7 | GCCGGACAGAAACTCGG |
| MI10 | 6 | 16308062 | TATTATATGTTCTGTGTAATACTCATTACTACTAT[G/T]TTTTGGTTGGATGATCATCTA |
| 25 | | 0 | CTGTTTCTCAGACT |
| MI10 | 6 | 16311400 | ATGGGTTTTTAGTGTTAGTAGATCTTTCGTGTTTA[C/T]GCTCTTTAGTTAAAATCTTGG |
| 26 | | 3 | AATTTTATCATATT |
| MI10 | 6 | 16325456 | CTCTCCCTTGGTTATTTGGAGGAGGACGAACCTTG[C/T]CCCGTCCCTGCAAGCCCAT |
| 27 | | 2 | ATGCTTCGGTGGTCCT |
| MI10 | 6 | 16411671 | TGCTCGTGAAGGGTTATCCTTCTAGTTCTTACCAC[C/T]GGCGTAAGGCTGTGTTTGTT |
| 28 | | 7 | ACGCAAAGCAAAAGC |
| MI10 | 6 | 16451456 | AACCGTCTATCTACAACCTAGGTCGGGACACATGA[C/T]GGAAATGGTGATGGCGACG |
| 29 | | 8 | ACGACGACGGCGATCT |
| MI10 | 6 | 16505370 | ATCCCATCCCAGGAGAAGAGGCATATTCTCCGGCG[C/T]AGGGAGGGAGCAAGCAAC |
| 30 | | 1 | TCAGGGTCAAAAAAAAT |
| MI10 | 6 | 16912422 | ATCTTAATTATTTCAAACATGGAACTAAAATTACT[A/G]ACGGTTAATTTGGTTCGGTTA |
| 31 | | 5 | TTCAGCTTTGGTTT |
| MI10 | 6 | 16930994 | GAAGGATACAACACTAGTGAGGAAGCATACAACCC[A/G]ATCCGAGAATGCCTCCAC |
| 32 | | 2 | GTCGACTCAGAGATCCA |
| MI10 | 7 | 1631705 | GGCATGAGGAAAGCTGCGTCCAGGCACCACAACCT[A/G]CACCTCCACCCGCAGGCA |
| 33 | | | AAACTAGCTACAACAAC |
| MI10 | 7 | 2563092 | AAAAGTCAATCAGAATAATTATGTGAAGGCAAAAG[A/C]AGTCCCAAAGGTCATACAC |
| 34 | | | ATGTACAGGGACTATT |
| MI10 | 7 | 3003531 | GGCGTGCAGCTCTAAAGACAACAAAGTCAGTCAGC[A/G]TGCAGGGGACACCCACT |
| 35 | | | GCTGAAAACTCATTGACA |
| MI10 | 7 | 3763473 | AATTGAGCACGCTTCTATCACCGCTCCACGTATAC[C/G]ATTCGCTCCAATCAAATCAC |
| 36 | | | GTTAGATTTATATTA |
| MI10 | 7 | 4083633 | GAGATTCGTCTCGTTGTTCGAGCAGAAGCCGAGCA[A/G]CCTCACGATGTAGCGGTGT |
| 37 | | | CGGATCCTCCCGAGGG |
| MI10 | 7 | 4835244 | GCGGCCATGGCCAGGTCTGATGATCAGTGCGACCG[C/T]CTTGTGTGGGCATTGCCCA |
| 38 | | | TGGAGCGTCCGCTCAG |
| MI10 | 7 | 7155293 | CCGTCGCTGCCCTACCTTCTCAGGCCACCATCGCA[A/C]CCTCACTCGCCACCGTCGC |
| 39 | | | GGCATCTAATCCCTGA |
| MI10 | 7 | 7917139 | ATCGTCGGCGACACGCTAGGTGCGTGCGTACGTGC[A/G]CAATTGCATATGCATGCGT |
| 40 | | | AGTATATATATAGCCC |
| MI10 | 7 | 8182989 | GTAATAGTGCTCCGCAGAACGCTGCAGCACTAAAC[A/G]GTTACCAAAATTTACCGAG |
| 41 | | | AAGCTCCAGCGCAAAT |
| MI10 | 7 | 9630005 | CATCATCTGAGTGCCTGCCAACAGCAGGGAGATAC[C/G]CTTGGCTCCAACCGCCACC |
| 42 | | | CATAAATCACACTCCT |
| MI10 | 7 | 9833298 | TTTCATGTAGCATAACCTTGCTAACTTCTTGTGAC[A/G]GGAGGGGTTGTGTGCGGTTC |
| 43 | | | GTGGGGGCACAGGGA |
| MI10 | 7 | 9919655 | TATGCCAGATACTGAAGTCAAGCAAATGCTCATGG[C/T]GAGAGGTACCCCTGATTCA |
| 44 | | | CCATCACTCACTTATT |
| MI10 | 7 | 10789564 | GAATCAATTATCCACCTCTTCTTCAAATGCAACTA[C/T]GCGAAGAGATGTTGGCAGAC |
| 45 | | | AATAGGAATCACTCC |
| MI10 | 7 | 10800312 | ATTCCGGTTGATTTGCAGCTTATTAATTGTACGGT[G/T]TTTTTTTACTCCTGGGCTTAT |
| 46 | | | CGATCTGTCACTTG |
| MI10 | 7 | 13862480 | GGCTGGTGTGGTGTGTGTGTGGACCAAATTTATTC[A/G]TAAGCATAAAATTCAGTTTT |
| 47 | | | CCAAACAACATTCCC |
| MI10 | 7 | 14080772 | GGTGGACTATTTTTTTGTCTCATGTTGCTCAGTCA[G/T]CTATGGTTGTGGTCGCTCCTC |
| 48 | | | AGTAACATTAGCGA |
| MI10 | 7 | 14876814 | ACAACTTCAAAAAGTATACCAAACGGTGGACTCTT[C/T]CATTGGCTTCTTGTAGCATC |
| 49 | | | CAAAATCCTGATTTT |
| MI10 | 7 | 15053002 | ACAACGCTATCCAAAGGATGCGAAGATCTGCATCA[C/T]TGTAGTCTTTAAGGCTCGAT |
| 50 | | | ATGCCGACTTCTTTA |
| MI10 | 7 | 17254591 | ATGAAGGAACTCTATTACTTTCTTAGATTCTGACA[C/T]TTAAAATGAACATGTTTATGA |
| 51 | | | TAACCGTGAATCAT |
| MI10 | 7 | 18152027 | TGTAGCAACCAACCTTTGCAAGAAAAGTAGCAGTT[C/T]AATTTTTTCCATTACAAGA |
| 52 | | | GGAAACAAAGTTGATG |
| MI10 | 7 | 18373857 | CTGAGCAAGTGTCGAGGTGACAATCACTCAACAGC[G/T]CGATCTGGACGGCCAGAA |
| 53 | | | GCTCCGGTCGCTTTCCT |
| MI10 | 7 | 20200790 | TCGGTACGTGTTCTGACGTGAGCACTAAGCCGGGA[A/C]CTCACCAGCATGTATAGTG |
| 54 | | | AGTACCTGTGTAGAAA |
| MI10 | 7 | 21288129 | ATGTTAAGGCCCTCAGATTCTTCACCGTAGTCCTG[A/G]TAATTGAAACGAAACAGTG |
| 55 | | | GAATGTCATGTCACCT |
| MI10 | 7 | 21649443 | CGAAACGAATTACATTTTTCATTTTTCAGGGGAAA[A/C]GTTGGTCTGGTGTGCGCTCT |
| 56 | | | GCTGGAAGCTGATCT |
| MI10 | 7 | 23549455 | ACATTTCCTTCCTTCACTTCAGACATGGTCAGCTC[A/G]CAGCTACAGCTGCACCGTTA |
| 57 | | | CGCAACGGGTCGCGA |
| MI10 | 7 | 25596939 | AATGGAACCTATCAGCTTCTTGCATCGTTCAATTT[G/T]CTATGATCAAAGCCAAAGCT |
| 58 | | | TACGTAGTTACGTTT |
| MI10 | 7 | 25905364 | AGCACCTCAAGCAGTATCAGTAGCATCAGCCACAA[C/T]AATACCAATATCTGCTATCT |
| 59 | | | TCAAATCAAGTGCAT |
| MI10 | 7 | 28566928 | TTTTGAATGCATAGAGGTGTTTGGTAACAAAATAT[G/T]CTCTTTATTTTACACCATGTT |
| 60 | | | CCATTTAAAATATT |
| MI10 | 7 | 30254596 | CTTGATCGTGTTCCCTACATCGTTCTCGCCATTTT[C/T]CCGAAGGGACCTGCTGCATG |
| 61 | | | CTCAGTTATGCCATA |
| MI10 | 7 | 31902459 | GTTGACATCTTCAGGAGTATAGTGGGATAGCTGCA[A/G]AAACCTGTCTCTATACTCAC |
| 62 | | | TTACTGACATGGGCC |
| MI10 | 7 | 32639248 | TGTTCATCATGTCCCTCTGCCCAACTAATCTGCAA[A/C]GTGGCATGGTCCCCCTCCAC |
| 63 | | | TCTTGGCTGCGGACC |
| MI10 | 7 | 33703826 | TTGAATGAATTTAGAAAGAGCACTAGATTTACAAC[A/G]TCAAAATAGTATTACTAGAC |
| 64 | | | ACATAAAAAATATTT |
| MI10 | 7 | 34646711 | ACTAGTACTACTACAAGGTAACCCATGGAGAAAGA[A/G]GAGCAGCAGGCCAATGAT |
| 65 | | | TATACCGACGAGGACAC |
| MI10 | 7 | 36525772 | TTGCTGCCTTATTGCTAAGGTGAGAAAAATCATTA[C/T]AGGTACTGTGTCCATGCCGC |
| 66 | | | CAAAGTGGTCGGTTG |
| MI10 | 7 | 36570698 | GGTCGACATAGCCAACATGATGGCACTCAGGCCCA[C/T]GTTGCCCGCGCCGTCTGTA |
| 67 | | | ATAAAGAATATGGGAC |
| MI10 | 7 | 40633183 | CGATCAAGAACAGGCAGGGATTAGTTGTAGGGTTC[C/G]AGTTCCAGAAGTAGCGAC |
| 68 | | | TTTGTTGTCTTAAGTAT |
| MI10 | 7 | 43097028 | TTCTGAAATCTGCGATCTGAAGTCATTGACCTTCG[A/G]CACCAAGCCATCGAGAAAG |
| 69 | | | ACAAAATTCTTCTTTC |
| MI10 | 7 | 47437259 | AGATTCTCCTAGTGTGTAGCCCCTTGTTAGGTTTC[A/G]CCAGTCATACAAACACAGTA |
| 70 | | | CTCCTCCCTAAGGTG |
| MI10 | 7 | 47954120 | GCGGGACAAGACAAGCTACGTACACGGTACGGCAG[C/T]GCACTGCATGTGGTGATG |
| 71 | | | TGGAGCCAGGCATACTG |
| MI10 | 7 | 51374727 | GGCACTTTATTAGATTGTGCAATATACAGTACTAG[A/G]AAAATGGTCTTTATATATTCC |
| 72 | | | TTTTTTCAGAATAG |
| MI10 | 7 | 51524634 | GTATGATGTAAATGCTAGGGCCATAGTTGAGTATC[A/G]ACCTAATAGGACTCATCTATG |
| 73 | | | TGGTGCCATCAACG |
| MI10 | 7 | 54428641 | AGCACCTTTAGGGATGAAGTTTAGGGCCGTGACTC[A/G]CCGGATTGCTTTTCCCGGC |
| 74 | | | CATGGCCTCCGTCGCG |
| MI10 | 7 | 57304397 | CGAAAAACGAAGAAATTGTTACGAAGGAAAAAACT[C/T]ATGCTTTGTATGCACCCCA |
| 75 | | | ATACCCATTATCGAAA |
| MI10 | 7 | 61704876 | GGGTTAAAACCTTCAAATTGAGTGAGGACATTTAT[C/T]CGGATAGGCTTACGCACCG |
| 76 | | | CGTTCGTATGTTCATC |
| MI10 | 7 | 65421941 | GCACGGCGGTGGGTTCACCGGAGCTCACGAAATCC[A/G]GTTCTCCCGAGCGCCATAT |
| 77 | | | CCAAATTCGCACAACC |
| MI10 | 7 | 70266107 | TGACGAGGTGGGCCCCCCTCCTGTTGCTGACGCGG[A/G]TTTGGCAAACGGACTGGC |
| 78 | | | GTTCGTCTGTTCATCCG |
| MI10 | 7 | 79667562 | ATAATAAAGTCCTCACCTTTAGGGTCGAATCCAAC[A/G]GTTGCTGGCGCAGCAGTGT |
| 79 | | | ATCTAGCCAGCTCGAA |
| MI10 | 7 | 81999752 | TTCGAGTTTTGTAAATGTCTGACTCATGATTTATC[A/G]TAGAGAGAGGCAGCGAGCC |
| 80 | | | ATGAGACCCGGTCAAA |
| MI10 | 7 | 82417512 | TTTCTCGGTACATTTCTAAAGTGTTAAAGGTGCTT[C/T]GGCAAAGACCACCTTCGGG |
| 81 | | | GTGAATCAGGAAAAGA |
| MI10 | 7 | 83062843 | GAGGATTGAACGCCTCTGAACGCAGCACATTGAGG[C/T]TGTGCGAGACAAATCAGC |
| 82 | | | CACGGAAAACAAGAGCC |
| MI10 | 7 | 83480640 | AATATGAGCAACGCTACATGGTGTTTGAATACTGC[G/T]AAATGCAAGCGGTGCTGGA |
| 83 | | | TATCAATTAATCTCCA |
| MI10 | 7 | 92993698 | TTTGTGCAGCCCATCAGTTGTCGCAAAAAAAGTTC[C/T]AGCGCCGGCTATGCTGTTG |
| 84 | | | GTTCCATGCCAAAAAA |
| MI10 | 7 | 94971993 | TCAGTTTGTTGATCTTTTCTCTCACATCCAGGATA[C/T]TGTTCATGATCCAATCACAAG |
| 85 | | | CAGAATGAATAACA |
| MI10 | 7 | 95247076 | TGGCTACGATAATCCATATTTTGAAAGCGAACTAT[G/T]ACATAATCATTTGGAATTGCA |
| 86 | | | TTGAGAAGTCCTTG |
| MI10 | 7 | 98888851 | GCATATGCATTCCTTGCACCAACCAATGGAGCCTA[A/G]AGGAATTGAAGGCGACTGG |
| 87 | | | AGCTATGGCCCTTGAA |
| MI10 | 7 | 99487571 | CCTGGTAACCAAAATGGCCGACACCCCTATTTTTT[A/G]TCAACATGACCAAAATCAA |
| 88 | | | CACCCAACCTTCTCTC |
| MI10 | 7 | 10032125 | ATGAACCAAACAGCGTAAAGGGATTGGACCACGTT[A/G]CCAAAGGCAACAAACAA |
| 89 | | 7 | AAATAGGTAACCATTGCA |
| MI10 | 7 | 10082681 | ATTTGATCAGACCGACGAGGCTACTATACAAATTA[C/T]GTAATCAGTAAAACAAAAAT |
| 90 | | 3 | ATAAACTGCCCGTCT |
| MI10 | 7 | 10160641 | GGCTAGATTCGACTCAAAGAAAAAAATGAAACATG[C/T]GATCTAATAATCAACAAAG |
| 91 | | 3 | TTACTATTAGCCCAAC |
| MI10 | 7 | 10184643 | ATCTTACAAGTATGGGCTAATGTGGTTAGTTGCTG[A/G]CTTGCTTTAAGATTAGTGGG |
| 92 | | 9 | ATGAACATGTTGTCA |
| MI10 | 7 | 10244787 | GTCAATGAGCTTAATAATCCTGCTAGAATTCAGAA[A/G]TATCCTCCTTTAGTCTGTGG |
| 93 | | 0 | GGGTGTTTTCTTTCA |
| MI10 | 7 | 10357046 | AACTCGAAGGACAGATGGCGTTATATTTGGGGCTC[C/T]GATATTTGTTCAACAAAAA |
| 94 | | 3 | AGACTTACAGACACCT |
| MI10 | 7 | 10368468 | TTTGAATGCAGCATAGAATTATTGACAACACTTAT[G/T]GCACTCGTGACATCGCAAGC |
| 95 | | 1 | CAGGGCCCATGATTA |
| MI10 | 7 | 10387893 | ATTCCCCTTGATTGCATCTATACGAGGGTGGTTTA[A/G]CCTAGCCCCAAACTAGATCG |
| 96 | | 0 | AGGTTCTTCCATCTG |
| MI10 | 7 | 10666435 | TCACGAGCATTCCTGCTCGGCTGCTCCTCCGTCAT[G/T]CGCCGCCTTCGTCCTCGCCC |
| 97 | | 2 | TCGCTCCTCTATCTT |
| MI10 | 7 | 10702074 | ATCGGCCCTTTGATGTGTGGTTTGAGTATTATGCT[A/G]TCTCCCCACCCACTGGTTTG |
| 98 | | 2 | GGTTTTGTGGGGATA |
| MI10 | 7 | 11008247 | AAGGTAATCGACACATACCTTGTCCTTTTTTGTTG[A/T]TCCTTAATCCAACTTTATCCA |
| 99 | | 9 | ATGTTGTAACCTGC |
| MI11 | 7 | 11080846 | TATAGAGGATGTATATAGAGGAAATAGATTTAATG[A/C]CTTGGTCGTTTGTTCCCTTCC |
| 00 | | 4 | TAACTCCTCCCCAT |
| MI11 | 7 | 11115537 | TCGGATGATTAGTCACTTGATGAAGGGAATGGTTC[C/T]GATACCAATTGAGAGCACCT |
| 01 | | 1 | AGAGGGGGTGAATAG |
| MI11 | 7 | 11143588 | AATGTGATAAAGGGTGAACCGTTGATCACGAAGGG[C/T]CCAAGGCTGACATGCATGT |
| 02 | | 1 | TTGCTGACGGATGTCA |
| MI11 | 7 | 11256925 | TCTCGTCCGCTTGTAACCCCTACTACGAGCAATCA[A/G]CAACAGGGAGTCGGTAGCA |
| 03 | | 3 | CGAGCCACTAAAGACT |
| MI11 | 7 | 11296412 | GTTTTGGGGAATGAAAATAGTGAAGACAGTATTGC[A/G]AAATCGAATTGGAGATGAA |
| 04 | | 1 | TTTGTGAATGAGTGTA |
| MI11 | 7 | 12008661 | GTTGAACTCTAACACCACGAGCACGATCGACCTCA[A/G]CAACTGTTTTCAAGGCTCA |
| 05 | | 3 | CCCGAGTCCCGAGGTG |
| MI11 | 7 | 12143169 | AGCGTCCATCTTCTCCAGCATACAGTTAGTTATTA[A/G]ACCAACAGCGCAATCCAGCT |
| 06 | | 0 | TCTTTTTTTTTAGCC |
| MI11 | 7 | 12143810 | GGATGCTGTGGCTCCTTAACCTGCAGCACAGCTTT[A/G]CCGGACGCAAGTTCGAGC |
| 07 | | 5 | ATGCACTGAAGCATGGT |
| MI11 | 7 | 12180549 | AACCTATTCCGTTTCTCGCTCTGTATCGATTCTAG[C/T]TTCTCCTGTCGGTGATGGTTC |
| 08 | | 4 | TTATGGCTAGCAGG |
| MI11 | 7 | 12589885 | TAGCTTATTCTAGCGCAGTCAAAGATTCACACTGA[C/T]TTGGGTTCGTTCTCACTGAA |
| 09 | | 2 | CCTTAATCTATTAAT |
| MI11 | 7 | 12649958 | GTTTTAGTCACTGCAGCTGCTGGTGGAACAGGACA[A/G]TTTGCTGTTCAGGTGAGCT |
| 10 | | 6 | GATGACACTTATGAAC |
| MI11 | 7 | 12712959 | TGGAGAGAGAGATGCAGTTGGTTCCACTTCCACCC[C/T]GCTTCTGAAATAGTCTGAT |
| 11 | | 2 | GGAGACGTTATAGTAA |
| MI11 | 7 | 12845372 | ATGCGATGCAGGGGGGTTTTATATAGGAGTGCTGT[A/G]TGTGTGCTGGTTGTGGAGG |
| 12 | | 1 | TTCAAAGCCGGTTAGG |
| MI11 | 7 | 12946783 | TGTAGAATATTATAAAATGGAGAATTGTGGGACCC[A/G]CGAGTGGTGGATCTAGAAA |
| 13 | | 0 | TTGGTCAAGACCTGGG |
| MI11 | 7 | 13027840 | AAAAAAACTCTCTGCGCACGGACCGATCACTCGTC[A/G]TTCCGACGCTGACGACGG |
| 14 | | 9 | CGCATGTACTACCGGTA |
| MI11 | 7 | 13114279 | TCATCAATCAGTCTGTGTTCTTTGCAATGGTCCCT[A/G]GATTACCTTTATGCCTGCAGT |
| 15 | | 3 | GCCCTTATGCATAA |
| MI11 | 7 | 13114346 | ACTATTCTATGGCCTTGAAGGTGCACCCTGCAAGG[A/G]GCTAATATATTGCTTACCAC |
| 16 | | 7 | CGGCATTATCTGACG |
| MI11 | 7 | 13220090 | GGCATCATTTAATGTTTAAGATCTGCTTTTCATGA[A/G]AGCAGGGTCAGTAATAGAGT |
| 17 | | 6 | CAAGTGTTAGTATAC |
| MI11 | 7 | 13226175 | ACAAGCCTGTTTGGGAGCGCTTTTCATCTGATTCT[A/G]GCAAAAAAAAGCGACAGAT |
| 18 | | 0 | GCTCACCAAACGCTTC |
| MI11 | 7 | 13242237 | TTTAATTCCTCTCGGCTAGATATTATAATACGGCG[C/T]CTCAAACGCCAGATCAACCA |
| 19 | | 4 | ACAAAAAAAAACTCG |
| MI11 | 7 | 13341259 | TCTGCCTTCTAAACACTCCTCGTTTCCTCTCCAAT[C/T]TCCCCTACTGGCACCAAAAC |
| 20 | | 6 | CCGCTACGGGTTTCA |
| MI11 | 7 | 13398197 | TGGACAGTTTTCTCGAATTTTTTACTTTCTCAGGG[A/G]AAAAAAAAGCAGTACCAGG |
| 21 | | 1 | AGCACATCATCATCCT |
| MI11 | 7 | 13737299 | TCTATTTATTTGCAAAACAGCATGTCTCGTCACAT[G/T]TTTTTTATTTATATGTCATCT |
| 22 | | 5 | TCTTTCATGTACTT |
| MI11 | 7 | 13963899 | TCTCAAAGGTACTCTTGCTACATGCCGAGGGTAAC[A/G]GGATAATAGGAGTAACCCT |
| 23 | | 7 | CTTTAATTTCTGTGCT |
| MI11 | 7 | 14366827 | GTAGCAACAGGATTAAGTCAAGTGGAGCAGAATTG[A/G]AAGTTCGGGGCTGACAAT |
| 24 | | 0 | AAAAAATAATGGTACTA |
| MI11 | 7 | 14426484 | TTCGCTACCTGCAATCGTCTGATGAGAATATATAT[A/G]ACGAACTTTTAGTCTCTTCTG |
| 25 | | 1 | TGTATGCATAAGTT |
| MI11 | 7 | 14500928 | CAATGTTGTGCTACTCAACTAAAACAACCACAACG[A/G]TGCTATACCTTTAGCGATA |
| 26 | | 4 | GTTACATTGACTCCGG |
| MI11 | 7 | 14525846 | GAATGGTCGAAGACTATCACACTTGCTCAAGGTTG[A/G]TTTACTCTTTTTAAGCTCTC |
| 27 | | 0 | TCTAGCTTCTACAAG |
| MI11 | 7 | 14541662 | ACCATCTTCACCAACCAGCAATGTGTGAGACAATA[A/G]TCATGTCGCACGATTCTAA |
| 28 | | 0 | AATTACAAGGGTCATG |
| MI11 | 7 | 14581650 | GCAGACAGTCTAACAAGAGAAGGGGAAAAATCCAC[C/T]GATATCTTCTAAATAGCCA |
| 29 | | 2 | GGCCAGTTGGGTGGAC |
| MI11 | 7 | 15154752 | ATTCACGGGCAGTTATTAGCAAGAACGGTGTAGCG[A/G]AGCAGCTTTCAGTTGACCA |
| 30 | | 1 | TGAACCAAACATGGAG |
| MI11 | 7 | 15369232 | GGATGACCGGGACAGAGTGGAGTCGTGGGGAACAG[A/G]TATAGGGGATGGTCGATC |
| 31 | | 2 | TCCGATGTATGGGGTGT |
| MI11 | 7 | 15431261 | GAATAGACAAGAGTTCAGATGATGGTCGTCAATGC[C/T]CTGGAATACAATGAGGTCA |
| 32 | | 5 | CATGAAAACTTACGCA |
| MI11 | 7 | 15511109 | AGTACGCGATTCATCTCATCATCACGGCACCCGGT[A/G]GTGTCCATTATTGATTGATTA |
| 33 | | 4 | TTGTTCACCGAGGC |
| MI11 | 7 | 15582171 | TGACTGCACGAACGTATGAGATGAACTCACCAGAA[A/G]CGTAGCTCCACCAGCGGC |
| 34 | | 4 | GGAGGTGGCTGAAAGAA |
| MI11 | 7 | 15705400 | GGATACCAATACGTACATGTATGGAGGAGGATACA[G/T]ATAAATGAAAGGTTGTGGA |
| 35 | | 6 | GGACAAACATTAGTGG |
| MI11 | 7 | 16137891 | TTAGCTGGGCTAAGTTTGAGTGTTTGACTAGTTAG[C/T]TTGAGGCAAGTCCAAGAAC |
| 36 | | 8 | AGCTCGAGTGGGCCGA |
| MI11 | 7 | 16324576 | GAAAAATCTCTTCCCTCACTTCACGCCATCACCAA[A/G]CAGACGCTTGCTTGCTTCT |
| 37 | | 1 | TCAGAGCATATCTTCA |
| MI11 | 7 | 16423951 | TGTCCCATGACAAGTTCTGACCTGATTCAGCGTTG[C/T]TGCTTGCTGCTGATCATTAG |
| 38 | | 2 | TCGATCTGTTGCGAA |
| MI11 | 7 | 16482737 | ACTTGGCGGCGCCTCTTTCATTGGGGAGCTTTGGG[C/T]TTTTGGAGCCGCCGCGGTG |
| 39 | | 9 | GTGGTGAGGTTTCAGT |
| MI11 | 7 | 16487194 | ACAGGGGAAATGGAAAACTGCTACTGGTAGCAAAC[A/T]GAACGAGAAAGTCTGCA |
| 40 | | 1 | GCTAATAAGACGAGTCCA |
| MI11 | 7 | 16583383 | TGGTGTTGCCAGAAAGGTGTGTGCGTCCATCCCCA[C/T]GCATCGTGTTCGTCGACCA |
| 41 | | 0 | TGACTGCATGCCGTTT |
| MI11 | 7 | 16650079 | GTTGCAGTTTAAATGCAGATAGAACTCACGTAAAA[C/T]TTTGGAAATTGGAAAAATT |
| 42 | | 8 | TCTAGTATGCCAACAG |
| MI11 | 7 | 16727070 | TTATTAAGCGAGCTTAACCACGATTTTGCGGGGCC[A/G]TGGAGGCCGGCGCGGGATC |
| 43 | | 7 | CCGTGGGTGCCAACGT |
| MI11 | 7 | 16753919 | GCAATAACAAAAACTGCGCAACTGTGTAAGGACAT[C/T]GTCTGATACTATTTGCTTTC |
| 44 | | 7 | AAAGAGTATTAAACG |
| MI11 | 7 | 16787730 | GAGAAAAGAAGACTTGTCCTTTTCCTGACTTCTTA[G/T]AACTGGGCATTTTGGTGGC |
| 45 | | 7 | TGTGTAGCTTATCTTC |
| MI11 | 7 | 16920525 | ACAAATGCGGAGAGGCTGCTTGAACCCATGACATG[A/G]ACAACTCTCACCGGGCCC |
| 46 | | 1 | ACGCTTCAGTTTTTATG |
| MI11 | 7 | 16949375 | CGCAGTTTTTATCTGCTAGCACAAGCATTCACCTA[C/T]GCGTTGTAATTGCTCTTCTG |
| 47 | | 7 | GCTTCCCCGCCTTTT |
| MI11 | 7 | 16978995 | CTGATCTGGCACTAGCAGGTCCTAAAAGGCGAACA[A/C]AACGAACTGATAGATGGC |
| 48 | | 0 | ATACATGTACCTCCACG |
| MI11 | 7 | 17028367 | AGATGAAAATGGATAAGCTATTATTTTTAATTTTT[C/T]CTGATCCGCTCTAATTCATGT |
| 49 | | 6 | GTAATAAGAATAAC |
| MI11 | 7 | 17066494 | AACAGGATAGAGCTACTCGAGAAATGATAGGTGCA[A/G]CAACAATAACTGAGAGGG |
| 50 | | 1 | CAGACCAGAGAGGCAAC |
| MI11 | 7 | 17171761 | GTCAATGGCTTTAAGAGAAGGCATCACCGAATCCG[C/T]GGATTCTATAACCCATTGAA |
| 51 | | 9 | AGTTAAAGTTATTCT |
| MI11 | 7 | 17190185 | GAAGCTTCAAACCGCCTTGGCCTCCCTGGATCTGG[C/T]ATTGCCTGACACAGTCATG |
| 52 | | 3 | CCACGCTCCTCGTCGG |
| MI11 | 7 | 17197485 | CATAATCAAGTAGCTAGGAAAAATGGATACCAGAA[A/G]ACTTTTTTAACAGAATACC |
| 53 | | 9 | AGTAGATAATGAGCAG |
| MI11 | 7 | 17208487 | TGTATCCGTGTCTACGCAAGCAAAGCAGAGGGACG[C/T]GAGTCAGCAGGGTCGCTG |
| 54 | | 2 | AGATTTGGCGCCCGAGA |
| MI11 | 7 | 17224018 | TTTTGTCCATCCTTTATTCCAATTGCACTCATGGT[A/G]GGAGAATGCTAAGAGGTATTT |
| 55 | | 0 | TGGTCCTATTATGT |
| MI11 | 7 | 17309570 | ACATATGGAATTTTGGCACATTTCTCTAAGTTTTT[G/T]ATATATTTGGTGTGGATAGGA |
| 56 | | 6 | AATGATGGACCACT |
| MI11 | 7 | 17319159 | CATGTGGTTGCTACTTGCTAGAGTCCAAGGGCACA[A/G]GTCCATGCGTTTCAGACCC |
| 57 | | 4 | TGGAAGAGACACAAGG |
| MI11 | 7 | 17414550 | CTCTTCTACCGGTCAGTAGTCCTCCCATATTCTTG[A/C]GCACACAAGCACTCCTCCCA |
| 58 | | 8 | GCTCAAAAGAATACC |
| MI11 | 8 | 561450 | AGCTTGGTGCAGGAGAAACCATCAAGAACAAGCTT[A/G]CCTACGGCATCTCTCAAG |
| 59 | | | ACCTCATACTCCTCCGT |
| MI11 | 8 | 909568 | TCTAGGGTCTTTAGGGAGATCCTGAGTATCTCATC[A/C]ATCTAACAAATCTTAGCAAT |
| 60 | | | GGGATCAAAATCTAT |
| MI11 | 8 | 2262815 | TCACAAGACGGTGATCTCATCTCATGGATCCGTCA[C/T]CACGCGTATTTGTGATTAGG |
| 61 | | | ATTAATACAGCCGTC |
| MI11 | 8 | 3573434 | TCTGCGACTGCCTCTGACCGCTGACCGCACTGCTC[C/T]CTTCTCCTGTTCTCTCTCCC |
| 62 | | | CACTTCGAGACTTCC |
| MI11 | 8 | 5137158 | CTGTCCATGAGACGACCGACAGTTGACACCTGAAT[A/G]GAGTTATGCAATATTCCTC |
| 63 | | | AGCGTCTTACTAAGAA |
| MI11 | 8 | 5598245 | ACTATTTAGAGCATTTTGCAAACCGCGACAACAAG[A/G]GACTAAAAAAAAGCAAAT |
| 64 | | | TTGCCGTCTGCAAAACC |
| MI11 | 8 | 7307424 | TTTTGCGTACTCACCGGACAGGTTCGGTGCCATCT[C/T]AGAGATAATTGTTGGATAAT |
| 65 | | | TAAAGATGATATACC |
| MI11 | 8 | 7744350 | TCGTTCAACACTCTCACTTGTGGAAGTTGGTCAAG[A/G]GCCGAAGCAGCAACCACT |
| 66 | | | ATGATTAACAAGGACAT |
| MI11 | 8 | 7791757 | GTCCTGCTGGACTATGGAGTATCCTTGGACTTTCG[C/T]TGTGAATCGATTTTTCTTTGG |
| 67 | | | TGGGCGCTTCGGAC |
| MI11 | 8 | 7793262 | GGCGGTTTTACACGGGCCCAACGTCTTAACAACGC[A/G]CTGGTGGCGACGTGAAGC |
| 68 | | | CTTCGTACTCCACGCGC |
| MI11 | 8 | 9899035 | GACCACCTTCGCTGGAGCCGGCAACGGTCTCCTTC[G/T]GCATCTTCCAGTCGCTCAC |
| 69 | | | AAGTGGCCACACCTTG |
| MI11 | 8 | 10763566 | AGGCCACTATGCCTCCTGCCCCACCCTCGTCGGCG[A/C]TGCCCGGCATACCGCCCTC |
| 70 | | | CCTATTAAGAGTGTAA |
| MI11 | 8 | 15104895 | TTTCAAATGATGGCTTGTTTTGGTGATTTTAGATG[C/T]CAATTTAAAATATTTTAAGGG |
| 71 | | | ACAGTGCTATTTTA |
| MI11 | 8 | 16347015 | TATCCACACCTTCACCAGATGGTGCTCGAAACATC[A/G]CCGTAGATTTCGTGAGAGT |
| 72 | | | TGCTATACATAGCTAG |
| MI11 | 8 | 16425161 | AAACGGCGAAAGGTTTATAGATCTTAATACGACGA[C/T]ATTAGCTGCATTATTTGAAA |
| 73 | | | CTCATGCTAAGCGGA |
| MI11 | 8 | 18322354 | ACAGCCAAACTGCGTAGATAACCAGCTAATTAGAT[C/T]AGGGAAAGTAAAAGAGAC |
| 74 | | | AGAGCGAAGGAGCTCAC |
| MI11 | 8 | 24128220 | TTCCATTGAAGTGAAACAGGAATAATTAAGCTCTC[A/G]GAAAGAACAGGCCGACGA |
| 75 | | | CCTTCGGTTCAGCAACA |
| MI11 | 8 | 24353321 | AATTTGAGACAGAAATCAAGAAGCGTGGGCATGAC[A/C]GGCTCGTCGTCCATCCAG |
| 76 | | | CGCTGCTCAGGACAAGC |
| MI11 | 8 | 26168961 | GATTAGTTTGGAACTAAACCAAAGTTGATTAACTC[C/T]TTGACAATCACTCATCATCG |
| 77 | | | TAGACACATCATAAC |
| MI11 | 8 | 29996472 | TTGTTCCACCATTATATTCTGTCAGTTTGGCTTGT[A/G]TGTACGATCGACATGCATGCT |
| 78 | | | AAATCCTTCCTTCT |
| MI11 | 8 | 35003543 | ATACATCAAGGGGAGAAGAAGAACAAAATAAGAAG[G/T]ATGGCTAAGGGATACAAG |
| 79 | | | CAACAAGGCCACCACAT |
| MI11 | 8 | 40262882 | TTTCGTCTACATCCTGTTGTTCGTCGTCTTCTCGG[A/C]GACGCGTGTTGTTGCTCTTC |
| 80 | | | TTCCCGAAGACCGTT |
| MI11 | 8 | 44527180 | GGACGCGGGAGCTCGGTGGGAGAAGAACAATAGTG[C/T]CGCAGGCCCAGTGGTCG |
| 81 | | | GCGCCAAAATAAATGAAT |
| MI11 | 8 | 44829086 | TGGTTAGCACATAAATGAGTTAAGGACTCACATCC[A/G]GCATCTGCAAGATGAGCTT |
| 82 | | | GCCGAACGAGATTTGA |
| MI11 | 8 | 45386174 | AAGGGGGGCGCATGATCTAACCTGATTCGTTCATC[A/G]AGATATCGACGACTTCCACT |
| 83 | | | TCTCCCCCACCCTTC |
| MI11 | 8 | 46360391 | GTTTATTTTAAATAACAAAGATCCCCATCGGATCC[A/G]CCCGATTCAACACTAGGGGC |
| 84 | | | CTAGATTCGATCCTC |
| MI11 | 8 | 53355107 | AACAAATTATCGGCGTGCAGAGGATCTGGAGAGCT[A/G]GCGAAGGTATGTGGCTTGT |
| 85 | | | GGTTCATGAACTCCCG |
| MI11 | 8 | 53405933 | CCGTGCGATAGAGATCGAACGGTGCACATCAACAC[C/T]GGGAGGGCTGATGGCTGC |
| 86 | | | GGGCGGCGCCGCTCCTT |
| MI11 | 8 | 53612617 | TCGACATAGGGCCTGCTCGCGGCGAGCTCGGCCGG[C/T]GAGGGATTAGGATGAGGT |
| 87 | | | GCGGGCCAGTTACTTGA |
| MI11 | 8 | 53937902 | TTAGTACAATAGGTTATTAGAGATGGCAACGACTG[C/T]TATGCAAGCAGTGCTTCTGT |
| 88 | | | GCAAGCGTGCAAGCA |
| MI11 | 8 | 59652747 | ATTAACATGTAGACAACGTCGTTATAAATCTAACG[C/T]AATTTGAACGGTGCGAAACG |
| 89 | | | GACTTAAAATACAAA |
| MI11 | 8 | 65612115 | TGTGTGAATAGTTTGATAACAGAGAAGAAGCGCAA[C/T]ATTAATGAACTGCAACTGA |
| 90 | | | TAATATATAATATCAG |
| MI11 | 8 | 65809709 | TGTAATTAAACAGTAAAGCAAGATCACTGTGCGTG[A/T]ATTGGGCTTACCAAACTTC |
| 91 | | | TTCTGGGCCAGTAATG |
| MI11 | 8 | 66870876 | GTCTTCATTGCTGTGCAGCTATTTCATGTCAACAA[A/G]GTCCCTGAAGCACTTCCCGC |
| 92 | | | CATGTCGATCACCAT |
| MI11 | 8 | 67491530 | TCGCTCTCTCTGCTTCTGCTTGGAGTTGAAAGCAA[C/T]TGTATTGTATTCAACAAAAC |
| 93 | | | GAGCCGCAATGCGTC |
| MI11 | 8 | 68201240 | TTAGTGCCCAACATTGCCACACCATATGTTAGTCA[G/T]ATTATGCTTTCTTAGACAAC |
| 94 | | | GAGTTAGGTTAATAC |
| MI11 | 8 | 71527331 | GACCCTTACCTCCGCGCATCATTATTGGCGCCGAG[G/T]TTACACGTTTCGGCACCAAA |
| 95 | | | ATAAATGGCGCCAAG |
| MI11 | 8 | 71817174 | GAGTGGGGTGATGGCTGGTTTGGTGAGAACGATGA[A/C]TCACCGCCAGACTTTGGA |
| 96 | | | GACGACGAAGTCAAGGT |
| MI11 | 8 | 72292085 | CCATTCACTACTGACTGAATAATAAGCTCTTAGCC[A/G]GATGTTAGAAGACACATATT |
| 97 | | | TTACTTAATTAAACC |
| MI11 | 8 | 75162960 | CACACAGCCACTTCCACCGTCCGTCTGTCTGTCAG[C/T]GGTGTGCGCCTATTGCAGG |
| 98 | | | AAATTTCATCGGCGTA |
| MI11 | 8 | 75409846 | ATTTTGAACCTGAGTAAGCAATTCAAATTACGCAA[G/T]AGGTCAACTCCACGTAATTT |
| 99 | | | AGACCCCACCACCAC |
| MI12 | 8 | 75559898 | CGATGAATACAATAGGATCTGGTGAGGTGGATACA[C/T]CAAATATATTACAATCGCAC |
| 00 | | | GTAAGTTCAATGCTT |
| MI12 | 8 | 76379246 | TCGCCAGGAAAAATAAAGCGGATCACCTAGACGAC[C/T]TCGCAGAGACATTTGCAA |
| 01 | | | ACATGAGGGACGCTCGA |
| MI12 | 8 | 77073467 | GTCTACTATGCCGCACTTAAAACCCAGGCTCAACA[C/T]GGTGGTTGGGTTGAGCCTA |
| 02 | | | ACTCGTCATGCTGCAC |
| MI12 | 8 | 80021659 | TCTATGCCATCCGGACACAATTTCTTATTCTTCCT[A/G]CCATTGCCTGTGGCATAAAAG |
| 03 | | | GGATTGGGACCTGC |
| MI12 | 8 | 80767553 | CCAACGTTAGCTGGGATTTCCCCGGTGAGATTGTT[C/G]ATGCCCATGCCGAGGTGTG |
| 04 | | | TGAGCGCTGACATGTT |
| MI12 | 8 | 81285916 | CAATTCTAGCATCGAAGGGTGTTATGACGCCTGCT[A/G]CAGATGTGGCTATACCAAG |
| 05 | | | AAGTCCAAAGGTTAGA |
| MI12 | 8 | 83917520 | CGACGATCAGGATTAAAACGATGAAGGGGTATCCT[C/T]TGGTCCAATCTAAGCCGTC |
| 06 | | | GAAACCCAAATCTACG |
| MI12 | 8 | 84511152 | CTCTCACATCCCCCTTGCACCCTTCTCTTCCTCCT[A/C]CATCATCATCGACGATGGGTC |
| 07 | | | GGTTCCCATCGTCA |
| MI12 | 8 | 89846444 | AGTCCCTAAGTTGCTGTCCAGGATGAAAGCTCTTT[C/T]AGATTCGTCTAGTGAATCTA |
| 08 | | | CATTCCATGAATAGG |
| MI12 | 8 | 90089863 | TTATTTGTAATATAGTAGTGTTAACTCTTTTTTTT[G/T]GGTCAAATTGTATACACATGA |
| 09 | | | ACTACAAGATATGT |
| MI12 | 8 | 95162209 | CTTATACGTACTCCTGTTAACAGTGGACGGTCAAC[G/T]GTGTGCCGGAAGGCTTGAA |
| 10 | | | CCAACGCGGGATGAAT |
| MI12 | 8 | 95686554 | TTGTGTTGTAATAGCATAGAGTAAATGTTCTACAC[C/T]AACGTGTACTGCGCCACTGT |
| 11 | | | CAGTGTACGCCATCA |
| MI12 | 8 | 97299686 | AGCCTTCGCTTTTTGCCGTGAGGTAGTGTACGTTC[C/T]GTTGTCTTGGAACGAAGTC |
| 12 | | | CCTTTCAATGTTGTGC |
| MI12 | 8 | 99146526 | TCCCTTCTCTGCGGGCCCCGTCATTTTTACTTCCA[C/T]GTTGGGAACCCGCGTTGGCT |
| 13 | | | GTCAGGCCCAGCCGC |
| MI12 | 8 | 10136652 | ATGTGCTGCTGTTTTTTTTTCCAGCATACTCAAAT[C/T]CGTATTGTGGCCATAACTCTT |
| 14 | | 3 | AGCTCCGAACTATG |
| MI12 | 8 | 10170877 | ACGAAGTCATAGACTTACAGTATACTCATGGCCTA[C/T]TGAGCCTAGACCTGAGTCTC |
| 15 | | 2 | CTGACGACTTAAGGA |
| MI12 | 8 | 10177543 | CCTTCCTGCTCTACGAAACCTTGAAGGTAGGAGAG[A/G]CGAGGGATATGGGTCCCTG |
| 16 | | 3 | ATTTACTGCCTATGGT |
| MI12 | 8 | 10277579 | TCATGAAGTAGCCTGTTGTTTCAGGGAAATCCTCG[A/G]CGGAGACCTCCTGCTCCGC |
| 17 | | 1 | TGGCTTTGTCGGCTCC |
| MI12 | 8 | 10283300 | CGAGAATATCTATCCTCACTATTGGTCTCATTTTC[A/G]CTACTTCGTTAGGCACTATGG |
| 18 | | 2 | AATCTGTTTATGGA |
| MI12 | 8 | 10422115 | CTGTCAGAAAAAGGAAGGTGTGGACGTTCTACCGA[C/T]CCGACTGGACGGTCCGCG |
| 19 | | 8 | GCCTAGCAACAATAACA |
| MI12 | 8 | 10639899 | CTGTCGGATCAAGATCTAACGGCCGATCTCGCCCT[A/G]TACCGCTTCGGCAATGGTAT |
| 20 | | 5 | TTTGCCCCTGGAACC |
| MI12 | 8 | 10679391 | AACATAGCTGCTTCTTCATGAAGATCATCACTGGA[A/G]CTATCATCATGACTTGAGCT |
| 21 | | 6 | ATCAATGACTTTCTT |
| MI12 | 8 | 10720601 | GGGGAGGTGGAGAATGTGAAGTGAGTCTTGTCAAA[G/T]ACAATGTGATGAGAGATG |
| 22 | | 2 | ATGATCCTATGGGAGGA |
| MI12 | 8 | 11051527 | TCCACTTGGTGAGAGTGCGTCACAAAGGACTGAAA[G/T]AAGAAACAACGAGAAAC |
| 23 | | 9 | GTGTGAATCGCCCATTAG |
| MI12 | 8 | 11259768 | ATGAACAAGTATTGCATGACAGGTGTATTAAGTGA[A/G]TGAAAACATATAAAAAACA |
| 24 | | 9 | CACAAGTCATCAAAAG |
| MI12 | 8 | 11435262 | GAATTGAATTTGTTAAAGACAGATAGGATAGCTAC[C/T]GATTCTAGAGGCACAGTTG |
| 25 | | 0 | GTGGAGTTGATGATAG |
| MI12 | 8 | 11488707 | GTACCGAAACAGAGGCAGATAAGGATCCTTTGGCA[C/T]CCAGCTGAGGGAACACTC |
| 26 | | 3 | AGAAGAAGCGTCGGATG |
| MI12 | 8 | 11493860 | GGAGTCTTCGTCTAGAGATTTGCTGACTGAGACGA[G/T]AGGCAAGAGAAGAGTTAG |
| 27 | | 1 | AGTACCGAAGCGAAGAG |
| MI12 | 8 | 11654559 | GCCCAACCGGGTATGAGAATGCTAGCAGTAGTAAG[A/G]TGTTGACACATTTTTGTCA |
| 28 | | 2 | CCAAACACTATAATGA |
| MI12 | 8 | 11696555 | ACTGGTATTGCATTGATGATGAGTACTCATTTATA[A/G]ACATGGTGCTGCAACTGTTTT |
| 29 | | 8 | GTAGTTGATCTACT |
| MI12 | 8 | 11805593 | AGAGTGAGGTGCAGATCTTACGAATATTGGAGGTA[A/G]ATATTCTCGTTGTTTGTTTC |
| 30 | | 4 | ACAAAATGTAACGTA |
| MI12 | 8 | 11830131 | TTGGAGTACAAGGCAAGATAATATGGCTTGGAGTA[C/T]AAGGTAATTACAAATCTAA |
| 31 | | 3 | AAGATATACCTAGATA |
| MI12 | 8 | 11957820 | TGATGCATCATGTGACAAATAAAGAAGGTCTGAAG[C/T]GTGTTGTTAGAGAAGGAGC |
| 32 | | 4 | CAAGGAATAATGTCAT |
| MI12 | 8 | 12013736 | CCGCAGACGACGCCGGATCAGCGTGGTAGCCCGAG[A/G]AGGCGGTGGACTGGCCG |
| 33 | | 0 | GGAAAATAAAGCAGGATT |
| MI12 | 8 | 12165724 | AGCACAATTTAGATCTTCAATTGGTTTGATGAAAA[A/G]GACCACATCATCTGCATAAA |
| 34 | | 9 | TGGAAACTCTACTAC |
| MI12 | 8 | 12373792 | CTCCGCCCATTTCTCAATTGATCCGTAAAATGCAT[G/T]TGGAATTTGTGCATCCAAAC |
| 35 | | 4 | TAAACTAGGAAACAG |
| MI12 | 8 | 12475688 | GTTTCGAGAGAGCGATGGCGATCGATGCGGATGCT[A/G]GCAGGGTGAACTCCGCAC |
| 36 | | 7 | GACTCGCGAAAGGGACG |
| MI12 | 8 | 12540902 | TTGGCTTTATGAGGTGAAAATATGGGGATTGTGGT[C/T]GGAATCGAAGAAAGTGTAG |
| 37 | | 2 | ATCGATTGAAGTACTA |
| MI12 | 8 | 12562987 | AGCGCACGGGGCGCGGATGCGCGTGGCTGACAGGT[A/G]AGGCTAGGGTGGATGATC |
| 38 | | 4 | AGGATTAGGTACAATTT |
| MI12 | 8 | 12598927 | AGCTATGGAAACACAAGACAAACGGATCACAACGT[C/T]GTGCGCCTCATGTTAAGGT |
| 39 | | 4 | CATTTACTATAATTGA |
| MI12 | 8 | 12726396 | GTTCTTGAGGTGGTTGGCGACCACAAGAATGGGAT[A/G]TGTTGGTACTTGCTCTCCT |
| 40 | | 7 | GGGCAAGTGGATCCAA |
| MI12 | 8 | 12807645 | ATATTATTATTATATATTCCCTTTGTTAGCCTTTC[A/G]GTTTTGTAATGATTACACTAA |
| 41 | | 3 | ATCCTCCATCCTCC |
| MI12 | 8 | 12865510 | GTTTTATGCAGCCATAGCCATCTCATACGAAGCGC[C/T]CAGCTCAACTCATGCAGGCT |
| 42 | | 2 | GGAAATACCCAAACC |
| MI12 | 8 | 13106498 | CATTTTCTTTGAAGGCAAGCTCCGTTTGGTCAGTG[A/C]ACGTGTCCAAGAGAACCAA |
| 43 | | 1 | ACAGCGTATGGATGGC |
| MI12 | 8 | 13121438 | AAAAAGACGGCATCTTGTTCGGCCTTTTCTACACT[A/C]TTCTCACAGCTGGTGAATTT |
| 44 | | 4 | TTGGTTCTGCATTCT |
| MI12 | 8 | 13213376 | GTAGCCCCTTGTGGAATGAATTCATTTCTTTGTAA[C/T]GAGTTTAGATTGCAAAAAAT |
| 45 | | 2 | AAAATTGGGAGGTCT |
| MI12 | 8 | 13280677 | TCTCCACGGCAGTTGGGGGTATTACTGATCCACAA[A/G]CAGCTACGGCCTGGTTCGT |
| 46 | | 1 | TTGTGCTGGATTGCAT |
| MI12 | 8 | 13337200 | CACCTCTGGCCATGGCAATGGCGCCGGCGCCAGGA[A/G]GCGGGATGTTCCAGCTAG |
| 47 | | 8 | GTCTAGTGGACACCACC |
| MI12 | 8 | 13360080 | CATAATTTGACTTCATTTCTTTTGCCGGCATTTTG[A/G]TACCGCGAAGTAGCATGAAA |
| 48 | | 8 | AATACAGGTGTTCTG |
| MI12 | 8 | 13416685 | TAACATAGTGAGGAACTTGAGAATTTATAATCATG[C/T]GTTTAGCGACCTTATTATTAG |
| 49 | | 8 | TGCATAGAGAGTGA |
| MI12 | 8 | 13530553 | GTTGGCTGGTGGCACCAACTCATACACTGTTGATT[A/G]CTTAAAGAAAGCTGGTCTT |
| 50 | | 2 | TTCCAATCATCAAATT |
| MI12 | 8 | 13548730 | CTCTGTTAAAGAAGATTAGAAGAAACATATCCTCT[C/T]AACTTTTTTTTGAACTTCAA |
| 51 | | 5 | TCATAATGCTTCTCT |
| MI12 | 8 | 13708949 | CAAATCCTTCTGACAGACTTAATGGGTGCATGTTT[C/T]AGTTAACTGAAACTGGTTGC |
| 52 | | 1 | AGTGAATGGGGTCAA |
| MI12 | 8 | 13728672 | TCCCTTCCGTTCGCGCAATTTGGTTTCCTTGCTCA[G/T]GATTGTGCCTTTGAAATCGT |
| 53 | | 5 | CTATGCGGGCAACCT |
| MI12 | 8 | 13750458 | TACCACACCTCTCACGTAAGATGGCGCATAGGTCC[A/G]AAAAGGATCTCTTAGTTATA |
| 54 | | 6 | CGCAAACTATCATAG |
| MI12 | 8 | 13818134 | GTTGAGGACACTGAGAAGGTCAAGTCGCTGAGTGC[C/T]GAAGTTGACCGCCTCAAG |
| 55 | | 3 | GTCTCTCCTTCACACCA |
| MI12 | 8 | 13915711 | AGGCACTAGTTCCTCACGAATCAAGACACACTCTA[G/T]TTACTCCAGTTAACACAAG |
| 56 | | 5 | TTACTTCAGTTAAGCA |
| MI12 | 8 | 13963506 | CCGAAGGATTTTTTGTAGATAGTGAAAATGTGGTA[G/T]ATGGTGGAGATGAAGAATT |
| 57 | | 1 | GTTTGAAGATTTGGTT |
| MI12 | 8 | 14075496 | TTCACAAAATATCAAATTTCATACACCAGATTCAT[C/T]CACATTCACATCCACGTACAT |
| 58 | | 1 | TCTCAGCTGCAACG |
| MI12 | 8 | 14298608 | ATTCTCTGCCCCCTCTATTCTTGCAGTACATGAAT[A/G]CCTTGACATATTTAACAAGTG |
| 59 | | 1 | AAGCCTTTTCTAAT |
| MI12 | 8 | 14377509 | TCAATTCGTCATGTTTGATTGATTGCCCTAATGCC[A/C]TCAATCGAACAACAACTGTT |
| 60 | | 8 | AGCCGTCATCAACCC |
| MI12 | 8 | 14615922 | CGCCTGTTTGCTGCTCTTCACCTGGTTTCATCATG[G/T]GAAGCACGTACTTTTAGGCC |
| 61 | | 8 | ATCACGACACGAACA |
| MI12 | 8 | 14633216 | GCCTATTGCACCTTGGCATGCTTGGGAAGCACGTG[G/T]ACCTTGTCACAGGTCTTGC |
| 62 | | 4 | ACTACTCAGCCATAGT |
| MI12 | 8 | 15007793 | ACCTAAGGTCATTAAAGCATCGACAGAGTAAGGCC[C/T]CATTACGGTGGTGGCACCA |
| 63 | | 9 | GCGATGACATCTGGAA |
| MI12 | 8 | 15134615 | GATAAAATAAGTCAAAAAGTGAACCGTGGAACCTT[G/T]ATCACATCCAATCTAGGTC |
| 64 | | 6 | CCTGGCCTTGACGATG |
| MI12 | 8 | 15223402 | TTGGACGATCTGCAGCCAACCCACTGCCCTCGAAC[G/T]TCACGCACGATGTAGTTTT |
| 65 | | 9 | TAGTGGAGGAATAAAT |
| MI12 | 8 | 15378368 | ATATGTCAGGTACATAAGCTATCACGTGACACAAC[A/G]CAATTAGTACTAGGTAACAG |
| 66 | | 5 | AATTGCGGGCGAGGC |
| MI12 | 8 | 15547851 | GGTCGGACAGCGGGCTGGGGCCTGGGGTCATGCAT[A/G]CGAGTATGGCAAGTGGCA |
| 67 | | 3 | GCAGCAGCTACGAAACC |
| MI12 | 8 | 15662978 | GAGATTATGCATGGGCTGACGTGAGCGACGGAGCA[A/G]CGATTGTATATGATGATATC |
| 68 | | 8 | GAGCTGAGCCCGCGC |
| MI12 | 8 | 15887806 | TTAGGAAGAGTAAGTTTATGTTTTCCATTACAAAG[G/T]GGATCCTTTTTTATTGGTGG |
| 69 | | 0 | GTGGGTGGGTGGAAG |
| MI12 | 8 | 15924864 | CTCCAGATCCTGAAGTTTCGCATATAAGCCCCCCT[C/T]TTTCGTTCATTAAGGGTAAG |
| 70 | | 8 | TTCCTTATCTATCCA |
| MI12 | 8 | 15993863 | AATCACACAATTTTCAATATTGCCACACTAATAAA[A/G]ATAAGATGAAATGAGGAATA |
| 71 | | 0 | AAATATGATCTCCCA |
| MI12 | 8 | 16110739 | TGTGAACTGAGAACCTCGATCGGCTTTTGCCCTAA[C/T]AATCAACCTGATTTGACAC |
| 72 | | 7 | TAGCTGCATGCCTAGA |
| MI12 | 8 | 16132870 | AGTACGCCTCGGAATCAGAGGAGCAGCGAGGAAGC[C/T]GAAGGAGGGGCGAATCT |
| 73 | | 1 | AAGCCCAATTGAAGCATG |
| MI12 | 8 | 16177738 | TTAGATCATGTGAACTCGTAACTCTCTGTATAAAG[G/T]AAAAAAAAAACATAATGAA |
| 74 | | 3 | CTCCTGGCAAAGCGAT |
| MI12 | 8 | 16207874 | GATATAAAACCGGGCTAAGAGATTGGCAACACCCA[C/T]ATCCAGCATGCGTCAGATG |
| 75 | | 0 | TAGTACCTACCACGCG |
| MI12 | 8 | 16357270 | CAGATTTGGTGCCGGCAGAATAATGTCAAGGAAAC[C/T]TCATAGTAGTTTATTCGATC |
| 76 | | 6 | AGTACGCAGATGCAG |
| MI12 | 8 | 16440974 | CTCAAGTTCTACGTCGGGTACTCTGCGTGGGACCA[C/T]GACCAGCTGCTGAGCGAG |
| 77 | | 6 | ATCGACGCAGGTTACTG |
| MI12 | 8 | 16441796 | GTGTTGCTTTTTGCCGTTGGCTTCTGTCCTGCCAT[A/G]AATTTTCTACTGCCAGTACAT |
| 78 | | 3 | CATCGGATGCCTGA |
| MI12 | 8 | 16517923 | AGACGTTTTTTCTTCTTCGAATTATTGGCATCATG[C/T]TTATTGCTTTCAAAGTTCTTG |
| 79 | | 4 | GATCATACTACCAC |
| MI12 | 8 | 16578156 | TGGGAGACCCTTATCTATATACATGAATGAACAGT[C/T]GAACACATCAAAATTCTCTT |
| 80 | | 5 | GCAGTAAGATGAAAA |
| MI12 | 8 | 16601836 | TGTCGCTGAACTACAAAGTGGTCGTAAAAGGAGAA[C/T]ACAAACATCACTGCGATG |
| 81 | | 2 | ACACGCTCGCAGACGAA |
| MI12 | 8 | 16701394 | TTGCAATTGCAACAGAGCATCCACCATGGTTTCTT[A/G]CCTCCAGTATCAAAAAGGA |
| 82 | | 8 | AAGTAGCTTCAGCATA |
| MI12 | 8 | 16782261 | TAACATCGTATACGGATCTGGTTCTTCAAATCTAG[C/T]TTGACGATATAAACCAGCATT |
| 83 | | 2 | TCCTCAACGTCTTC |
| MI12 | 8 | 16896697 | GTGAAAGCAACTGGTACAATGCCATTTATGGTGAC[A/G]GAGACCAGGAAGACGAAT |
| 84 | | 3 | GGCGTGAACTTGAGGAA |
| MI12 | 8 | 17040587 | TTGCAAAACCACCATTAGCAACAAGATTCTTCGCA[A/C]AGCCTTTTTTTTCTTGGTAA |
| 85 | | 2 | CCAACATTGTTCGGT |
| MI12 | 8 | 17131421 | CTAGCACAAGCGCCATTCCCGCTGTACCGAAGTAC[C/T]AATCCAGCATAAATGGCAA |
| 86 | | 9 | CGAAAGCCGCATCCTT |
| MI12 | 8 | 17141016 | ATCTAGATCATACATACCCTAAGATTGTCATTGTC[A/C]TGTAACGCCGTAGCTGGCTAA |
| 87 | | 4 | TCACACGAATTGCA |
| MI12 | 8 | 17178628 | CGCGGCGCTAGTACTGCCTTTGTACGGCACATTTC[A/G]ATCAAAGGTGTGCTTGGCA |
| 88 | | 8 | TTTCGAGGAGAGACCG |
| MI12 | 8 | 17261601 | ACAAAATTCCCGTAAGTAATGAATTTCTCTCATTC[C/T]TTGTCTCATTGCCATTATTTT |
| 89 | | 7 | TTTCCATTGATACA |
| MI12 | 8 | 17361403 | CAGATCGAAATAGACTCCATGTGTGTAGGAGTAGC[G/T]ATAAATGTCAACTTGTAAA |
| 90 | | 3 | ACATGATCCCATCATC |
| MI12 | 8 | 17370661 | AATAATCCCATTGTGAAACAATGATTAATGGTGGC[C/T]ATGTTTGTTTTACCTGAGTTA |
| 91 | | 7 | TGTTGAGGAATTAG |
| MI12 | 9 | 478409 | GCTCAAATGGATGGGTGCAATTTTGTAAGTGACAT[G/T]ATAATAGATGGGCATTAACA |
| 92 | | | AGGTACTTATGATAT |
| MI12 | 9 | 623042 | CATTTATGACCGAAGGATCTATGATCAACGACCCG[A/G]GAGTTTAAAGTACTCAGATT |
| 93 | | | GCCCCCCATACACAC |
| MI12 | 9 | 676228 | GATGTCCTTCAAGAGAACATTAAGGTGGTGTACAC[A/G]CAATTTTGCAATGTGTATTG |
| 94 | | | AGCCTTAGGGGAGGT |
| MI12 | 9 | 1030551 | AGTGACCCTTAAATGCAAATAAACTGCGTATGGTG[C/T]GTCCCCCAAGTCCAAACTG |
| 95 | | | CATGCATGCTAAGGTA |
| MI12 | 9 | 1428595 | TCGTCCGAAGTGACGGTTCTACCCTCGCGTTTCTT[C/T]CACTTTTTGTTCTCTTTCCG |
| 96 | | | GTCGCCCACTGTTCT |
| MI12 | 9 | 1432544 | TGGTTCGGGCGGGCCAAGATATGTGTGCGGCACTC[A/G]TCTCCTTGCCATTGCTTGG |
| 97 | | | TTTTGGATAGGATCGC |
| MI12 | 9 | 2517288 | CGACTATTTAGTCCATTTCTACGCCTTTGATCGTA[C/T]GTGTGGCATGTGTTAATCTAG |
| 98 | | | GACCAATAATCTTC |
| MI12 | 9 | 2714062 | TTTTGCTGTTGGTGCCATATGGCGAGCCAGTAAGT[A/G]CTCGCTCTCCGGGGAGTATT |
| 99 | | | TGTGGCGAACGGCGA |
| MI13 | 9 | 5158723 | AAAAACTTCATCGTGTCTCACCTCACACCACATAC[A/C]TCTGCATGTCTTCTCCACCT |
| 00 | | | ATCTCCTCGCCACAT |
| MI13 | 9 | 6065561 | GCTGATCTTGCATTTTGAGAATTGTTGCCCCTGTT[A/C]TGCTTCTTGATCGTTTCCTCA |
| 01 | | | AGACTACGACCACT |
| MI13 | 9 | 6980945 | GACAGGGACTCTGATTCCACTGCGACATACTCAAC[A/G]TGATCTTCATTCCGTTGCG |
| 02 | | | ATCCAGGATTCGAGCA |
| MI13 | 9 | 7308828 | AGCGCAGAGCTAGCAAGTGACCGACCGAGAGCAAA[C/T]AGGCAAGGCAACGCAGG |
| 03 | | | CAAGATGGTGCAGCAAGC |
| MI13 | 9 | 7957610 | CTTCTACTTGTATCCTGACCTGCCTCTGCGGATAT[A/G]CCTATGCCTGGGTTTAATTTC |
| 04 | | | AGAGATTGCTTACT |
| MI13 | 9 | 8128069 | CACAAACCACCAAGCTATGATTGCCGCCTCAAGCA[A/G]TGGACGGCGGACATCTCA |
| 05 | | | AAGGATGTGCCGTACAT |
| MI13 | 9 | 8469608 | CCACGCTTGTGTCAATTGCGATCGAGATTTGCAAA[A/G]ACCATCACATCCTAGCTTCC |
| 06 | | | AATGTGGCAGCACTA |
| MI13 | 9 | 8973393 | AAATATAACTAAACTTAATAAATTGTTTGCACATA[A/G]CTGAATCCAATAATATACATC |
| 07 | | | GATGCATATATACA |
| MI13 | 9 | 9731764 | ACATCGTGGTCAATACCAATTAGCATGGTTTCAAC[A/G]CCACTGGTACTGACTAGTGA |
| 08 | | | CAGGTCCATCGATCG |
| MI13 | 9 | 9802449 | ATGAACATGAGTGTTTATTTATTCTCAGCACGGTC[A/G]ATATTTGTCATATAACTAATA |
| 09 | | | TATTGTACTTGCAA |
| MI13 | 9 | 10472496 | GGGATCGTCAGTTGAATTCGGTTTCTTCTTGACAG[A/G]CAAAAAAATGTTTCTTCTTC |
| 10 | | | TTTTTCGTTCTTTTT |
| MI13 | 9 | 10474313 | GACGAAATGACCTAATACAGGATTACAATTGTGCC[C/T]TTTACTGTTTACAAATTCCA |
| 11 | | | TTAGGGCAGTAGCGT |
| MI13 | 9 | 12348170 | CCTTGAAACACAAAAGCTCTGTCGAAATGCTGGAA[G/T]TTTGAGAAGAGAATCGAA |
| 12 | | | AATAGCATGCCTCGAAA |
| MI13 | 9 | 12875299 | AGACTCCAAAGAGATCAGATAGGACCGAGCTCCAT[A/C]AAAAAAACACCTGTTTCT |
| 13 | | | ATGCTTCCAAAGGCTCC |
| MI13 | 9 | 14207199 | CCGACGATGGGAACGCCGCTTTCTGGTACGCAAAC[A/G]CGGGTTCTGATGCGTGGAT |
| 14 | | | GCTGCCTTGTGTCGAG |
| MI13 | 9 | 15371489 | ATGTACAGGTTCCTCAATTCAAAATCACATACTCG[G/T]TTGCAAAAACACAACAGCC |
| 15 | | | GCTGTTCGCAATGCTG |
| MI13 | 9 | 16501382 | TCCTCTCTTACCAAGTTACCATTGCTATTGCTATC[A/G]TGGTTTAGGATCATTGGTGCC |
| 16 | | | TCTAGAGGTCTCCA |
| MI13 | 9 | 16684414 | GATTATTTTTCCCATCATTAGTTTGTTAACATGAC[G/T]ATCCGAGCTGGTGTAATATGT |
| 17 | | | ACCTAATATGAAGC |
| MI13 | 9 | 16689713 | ACCCTAGATATTGGCTCTTAGGGTTGATTTAGTGA[C/T]AAAGGGGATCACGGAAGATT |
| 18 | | | GGGAGAGATTGGAAG |
| MI13 | 9 | 17392234 | TACCATACATCCTCTTTTAATTGGATTGATGAAAA[A/C]AACTAAGTGTTCTTATGTACT |
| 19 | | | GTTTGAGCTTGCCT |
| MI13 | 9 | 17478403 | TTGTTAAGAATCAAAACAAGCAGACAACACGAGTC[A/G]CAATGACAACAAAATGTT |
| 20 | | | CAGACATAGATAACTAA |
| MI13 | 9 | 17649633 | AAAAATCCTCTTGCCTGTGACCTTCATCACAAACT[A/G]AACTTTTGGCAGTAGCAGA |
| 21 | | | TTGTAGGAGTTGGGCT |
| MI13 | 9 | 18628865 | ACACCTTGTGTTTGCTATGTCAGGGGAGGGGGGGT[G/T]CCATTCCAAAGCCATTTGA |
| 22 | | | AAATAGGATGAGCTTG |
| MI13 | 9 | 19238970 | AATCAGCACTGGACAAACGCATAACTTGAGGGCAA[C/T]GTCTCAAATGCAATTTCTC |
| 23 | | | AAGAGATGGCATATTG |
| MI13 | 9 | 20300953 | ATGAGAGGAAGTTGAGGAACACAAGACACAACAAA[C/T]AGCATAAAAATAGATGAA |
| 24 | | | TCACACGAAATGAAATG |
| MI13 | 9 | 20403999 | AGCACTGCGAGGAGCAAAACAAGGAATTACTTACA[G/T]CGAAACAGGACCACACTG |
| 25 | | | GTATTAATAAGAAAGAA |
| MI13 | 9 | 22110801 | GACTACACGTACGGAAATGCTACCGTACGTATAAC[A/C]CAAACACGACATGGACGGA |
| 26 | | | TGGACCTGTTGTGGTC |
| MI13 | 9 | 22490903 | GCGCTCCTTCTGCAGGCTTCCTTCTGCTGGAAATG[A/G]TCGGTGCTGAGCCTGCTAT |
| 27 | | | CAGAAACCTCCAGCGA |
| MI13 | 9 | 22792882 | CGACCACTACACGTAATACTTCTAATAACGCCAAC[A/G]TGCAGCGTCTCGCTTTTATT |
| 28 | | | ACAGTCTTGCTTTTA |
| MI13 | 9 | 23103530 | CCCCTTTTCCATTATCTAGGCATTCTAGTGAACCA[C/T]ATAAAATTATGCAATAAGGAT |
| 29 | | | TGGGCTTGCATTAT |
| MI13 | 9 | 23287230 | AATCCGAAGTGCTGCATTGCGCGCGCCTTTCTTCA[A/C]AGAAATCATCATCCTAGCCT |
| 30 | | | TTTAAGCTAGAGTAC |
| MI13 | 9 | 24964933 | GTACCCCACCAGTCGCTCATTGCGTTACGATCTAC[C/T]CACTGATGACAGTTCACGGT |
| 31 | | | GGAGTAAGAGCGTTG |
| MI13 | 9 | 27995821 | TAAAGATTGCTCCAAAGAGGAAGATACTATACTTA[A/T]GCAACAGCTATATATAGACG |
| 32 | | | ACGACGTTATTGATC |
| MI13 | 9 | 29222396 | GCCTGGACGAAGTATCTTTCGCTAGTTTGCTGGCT[A/G]GGCCAGAGGTAAGCCTGCC |
| 33 | | | TGTCTGTTATGGTGTT |
| MI13 | 9 | 31994112 | CCACGTTTTGACTCATGTGAATCGAATTGAAAAAA[A/C]AAGACAGGACACAAAAGC |
| 34 | | | TGAATTGGAACATAGGT |
| MI13 | 9 | 34213081 | TAATTCATTCAATCATCCAATTCAAAAATATGTTC[A/G]CATAACATATGATACATCACT |
| 35 | | | TGTGCCACCAACTA |
| MI13 | 9 | 35419946 | ATCTCGTAGAATTACTCGAAGTAAGGTTATTGTCA[C/T]GGAACCTCCCAAGATATTAG |
| 36 | | | GCCCACCTACAGATG |
| MI13 | 9 | 36918458 | TTGAAAGATGTCTTGGCTGCAATGAGAGCTCTCCT[G/T]AATTGGAGTTCTGTACTGA |
| 37 | | | CCTGAGGTTTGAAATC |
| MI13 | 9 | 39454399 | CGATGAAGGCACACTACTTCCTTCACTCGGTACTA[C/G]GGGGTAGGATTGATGAAAG |
| 38 | | | GAACTTGAGGGGGACG |
| MI13 | 9 | 41506113 | GATTCTTGTACTAAATCACATGTTCCAATCCTATC[C/T]ACGATGGTGTTAAAAACAATG |
| 39 | | | TAAGTAGTTTGATT |
| MI13 | 9 | 41752587 | ATACAAATCACGATTATAAAAAAGAAAGATAACAT[C/T]CGATCATAAAACTTGGTCCG |
| 40 | | | TAGCTATCATATAAA |
| MI13 | 9 | 42395700 | AATTAGATTTTATTTTGGGAAGCCAACGTTGCTAA[A/C]TTAACCATGAGGAACTTAGT |
| 41 | | | TATGATACTAAAAAG |
| MI13 | 9 | 43529871 | CGTAATGGGGAATCAGGGAATGGATCACTGGTGCC[C/T]GGTAGTATAATATATTAATA |
| 42 | | | GGTGGTTACTTAAGT |
| MI13 | 9 | 44287501 | AAAATAAACACATCCAAGACTTGAGAGAGGAAATA[G/T]AAAAGATGAAAACAAGTC |
| 43 | | | ACAATGATGAAGTCTCT |
| MI13 | 9 | 44448552 | GGTATAGCCATGGAAGCTTGTACAGCTTGCAGCCA[C/T]GCTTTCCCGGCTTCTCTATA |
| 44 | | | AAATGCAGGCACTGC |
| MI13 | 9 | 44481789 | ACCGCAGAGGTCGCCCCTACGCATCCATCCATCTT[G/T]TAGTGACACTCTCCCCCCTC |
| 45 | | | AATGAAGCGACTCCC |
| MI13 | 9 | 46067346 | GTCCGGCCGTGGCGCTGGCGTGGATTGGCACGCAC[A/G]GAGCGGCCAATAGTGTGC |
| 46 | | | CCTATGGTTCAGACCCG |
| MI13 | 9 | 48736962 | CTCATTATTCTTGTGGATAGCTACCTGTTCTGGAG[C/T]ATTGTCCCTGGCTTCAACCAC |
| 47 | | | TGCAAATGGTACAA |
| MI13 | 9 | 51635692 | GTGTTGTCTTAGTGACCTCGCTTTCTTCTCCCTCA[C/T]GAGACACCCTTGCAGTCGAA |
| 48 | | | CACACTCTGGCCATT |
| MI13 | 9 | 54923746 | ACTCAATTACTATGCCACACACCAAGCTCGCAACG[A/C]GGCGAATCATTCTAATGAC |
| 49 | | | TTGATTCATGTGTAAG |
| MI13 | 9 | 58147970 | GGCAGGACAAGACTTGCCATCCTCAAGTACAGACC[A/G]CGACGAAGAACAGCACG |
| 50 | | | CCACTGACAAGGTAGAGA |
| MI13 | 9 | 59257189 | GAAATCAGCTTAGCCGGTTTTTCTGCACAGCAGCT[C/T]GAGGCGATCCCAAAAGGTG |
| 51 | | | ATTTTTGGTTAAATCA |
| MI13 | 9 | 60412694 | TGGAGCGCCACTGGCGATTTTAAGATAAGGCACGG[A/G]CGATTGCTGCCTAGTCAGC |
| 52 | | | AGTCACTCATGAGCAC |
| MI13 | 9 | 63258589 | CGTGTTTCTCTACTTGACGGAAGAACTTGCCCCAC[A/G]TTCAACAGACGACGTGATT |
| 53 | | | TTTACTAAAAATATTG |
| MI13 | 9 | 64535376 | GTTCGGTTATTAGCCCACGCGTGCAGAAAGTGGAA[G/T]CAGACCAAGAAGTGGGAA |
| 54 | | | CCTAATTGCCTTGTTTG |
| MI13 | 9 | 65143208 | ATGCTTTGGATATTGGATTTGAATATGGATATACT[A/C]CTGTGATGTGGTATGCTGCTT |
| 55 | | | GTGCTTGGCTAATT |
| MI13 | 9 | 67923649 | GTCACTGAAAGGTGTTAAGGCTGCTGGGTATGAAC[A/G]GATGACTGCTGTTCAGGA |
| 56 | | | GGCTACTCTTCCTATTA |
| MI13 | 9 | 77479105 | CTCTGTGTTCGTAATTAATTTGCATTTGCTCTTGC[C/T]TGATCAAATCAAAAGCGATCG |
| 57 | | | ATAACCTATACACA |
| MI13 | 9 | 78560665 | CTCCATCCCTATCTACCAAAGCTAATGTTCACACG[A/G]TTTCCCCTAATTAATTGGTTA |
| 58 | | | ATACCAGTGTAATA |
| MI13 | 9 | 78707863 | CAGTGGTAAACAGAACGGTACATTGAATATTTTCT[A/G]GGAAAAAACTAGGCTATAC |
| 59 | | | ACATGATGAATACAGA |
| MI13 | 9 | 79061255 | AACCGGCAGAGCACTGGCACACATTATAAAATTTC[C/T]CGAGGCGACTACGGCGAG |
| 60 | | | GCATCGACTTCGCCATG |
| MI13 | 9 | 80060290 | AGTGTATTTGAAAATATATTAGGAGAGGTGGAAGC[A/G]CCGTTTTGAAAGTTGATGC |
| 61 | | | ATAGTGGAAGGATTGT |
| MI13 | 9 | 80077685 | TGTATATAATTGGTAGGAAACTTGGTCCAAAGATC[A/C]TATCTTCTAATCTGAGTTCTC |
| 62 | | | AGACTTTGTTCCAA |
| MI13 | 9 | 81196419 | AAAAACCGAGATGACAACACGAGGTTGAAGTGTTT[A/G]ATTGGAAGGTAAGTTAGT |
| 63 | | | GATAGGTTAGAGGTCTT |
| MI13 | 9 | 81588241 | TAAAATTTGAAGACATTTAAATTGTACGTGGGCAT[A/G]TAATTACCCACTGATAAAGC |
| 64 | | | GAATGACTGTAACAG |
| MI13 | 9 | 81819115 | TGCGCCCCGTCATGGAAGCTGTGGGCTGTGCTCAT[A/G]TCACTCCCCAAGTCCCTAG |
| 65 | | | ACCCTAGTCCTCGTTG |
| MI13 | 9 | 86236968 | GCTTGTTCGACTATTGACAATATCGTTGCATTCTT[A/G]TTTGCGACAGAGTTCTCGAA |
| 66 | | | GCAAAACGATCGGAT |
| MI13 | 9 | 89166548 | GAGTACCTCGGCGGCACTCCGCAGCCATTCGTCGA[C/T]CTCTCCACCAATTTCTACA |
| 67 | | | CCAGGTCCGTCCATCG |
| MI13 | 9 | 89759159 | AAAAAGAAAGACAAAATAAAACATAAATGTAGAGG[C/T]AGAATATACTAGTTGTGCA |
| 68 | | | TTAATTAATAAATGTT |
| MI13 | 9 | 92253578 | AACTAACCTCAGAACACCTGATATTCTCTCTTTAG[C/T]AGTTGGCATAGCTCACCCTT |
| 69 | | | CAGCCAGACCACCCG |
| MI13 | 9 | 92879547 | AAATAAGAGAAGCTTTACTTCAAGTATCTGATACC[A/G]ATAATGATGAGATAATAAGT |
| 70 | | | AGTGAGGCTAATTCC |
| MI13 | 9 | 94091703 | CCATCACGAGTAAATCCGGTGGCGGAGCCGGAGAG[A/G]GCTCGATGCCGATCACGA |
| 71 | | | GCGATCCAAGCATCCAA |
| MI13 | 9 | 95642497 | GGAGATGATCCACAGGTCCCACGGAGTCGTGGGCG[C/T]GTGCGAGGATGACTCAGA |
| 72 | | | GGACCCACACACCAGTG |
| MI13 | 9 | 96347765 | TCCCTTCAACCTTCCACCGAGTTTACCATGAAGCC[A/G]CGCGCATGCCTCATGACGG |
| 73 | | | ACTCGTCGTGGAAATC |
| MI13 | 9 | 96456258 | GATCAAGGCCTGACCTCACCTTGGAAGCTTGGATA[A/C]GAAAGCAGAACAACAATC |
| 74 | | | CTGGAGGCTGCTGAGTG |
| MI13 | 9 | 98516535 | ACCTCTAGACAAAGGCGACAACGTTTCTCGACGCT[A/G]GAGCTTCAAGATCCGGGC |
| 75 | | | GCGTTACAGCAAGATTC |
| MI13 | 9 | 98519851 | CCAGGTGGCACACGCGGTGATGCTGCAATCGTTGC[A/G]GCACATGACGATGTCAAG |
| 76 | | | AAGATTGCCGGAGAGGG |
| MI13 | 9 | 99367488 | AGTTCAAACACTATCTATTACCGAATGACTTCCAT[C/T]ATCCTGTAGTTCACCTACATG |
| 77 | | | CAAGGAGGATCAAC |
| MI13 | 9 | 10004550 | ACGCCCATTGATTGATGGAGGACTGACCCCTCGCC[A/G]TCTCGGTATCGACGCGCGT |
| 78 | | 5 | CTCGAGTCGGGGTGTT |
| MI13 | 9 | 10037552 | ATACCTCGATGTCTCTACCATTCCCTAATCCAATC[A/T]TAAATGATAATAAATAGTTTG |
| 79 | | 4 | AAAGGTGTCAAGTT |
| MI13 | 9 | 10044840 | GGGTGGAGCTTCAAGCCTATAGAGGAATCCCTCCG[C/T]GACACTGTTGAATCCTACA |
| 80 | | 6 | AAGCATTTGGCATCCT |
| MI13 | 9 | 10076738 | TTCAATTTTGCGCTTTGGAGTTGGAAATGAAATTT[A/T]GCATTCTTTCTTCTATGAATC |
| 81 | | 5 | AGGGAATTATGTGA |
| MI13 | 9 | 10597584 | AAAGCCTGACCTATTTTAGTCGCCAAAATAACACG[G/T]AGTAGTAGTAGCACACACG |
| 82 | | 4 | CATAGTGGCGTAATGA |
| MI13 | 9 | 10743074 | CGGACGCTGAAGGATCGGATCCCGAATATGCGGTC[C/T]CCTTCGATGTCAGGTATATC |
| 83 | | 5 | CTACGAGAAATGGCA |
| MI13 | 9 | 10803896 | TTTATTTCTCGTCGTAGAGAAAATTTATTCGCTCA[C/T]TGTATTAAGTAAAGATAAAGA |
| 84 | | 7 | TTTGATTCTCTCTG |
| MI13 | 9 | 10847740 | TGTTTTGGCACATGTGTATCAGGCGAAGGTTACTT[C/T]AACTTGGTCATGTAGCAAAG |
| 85 | | 9 | CCTTCACCCTCGCCC |
| MI13 | 9 | 10853269 | ACAAAGAAATGAAGTCATCAGAATTGTAGAAGTAT[C/T]TTACATACAAATAAATATTC |
| 86 | | 1 | TGTTAGGGAATCTGA |
| MI13 | 9 | 10854260 | AATGGATGGCCTACAAGCCACTAAACATATCCGTT[A/C]TTTTGAGAATACCGGCTGTT |
| 87 | | 8 | GGGACGTTTCTGTAA |
| MI13 | 9 | 10956439 | ATAATGTCAGACAGGTAATTCCAGCACACGTAAAT[C/T]AGTAATAAACACACATTCTT |
| 88 | | 6 | GCATGGACCAACAAG |
| MI13 | 9 | 11109064 | AAGATCAAACATATACGACGAATTAATTAACTTGA[C/T]CATTTTGAATTAAGTTGAATT |
| 89 | | 3 | CCTGTGTAACATGA |
| MI13 | 9 | 11303574 | AGTACTAGTACGCAGTTACTTCCCTTTAATTAATT[A/C]CATGGGTGCGAGATGCTGCA |
| 90 | | 6 | TTAGCATCTGTGGTA |
| MI13 | 9 | 11569224 | AGGCCCTTTACACCCACGTACGGCCCGGCCACCAG[C/T]CACCAGCCCAAAACAGAG |
| 91 | | 5 | CTCTCAAATCGAAACCC |
| MI13 | 9 | 11766068 | ACATGAAATCTACTTTTTGGAGTCAAACACTTCTC[C/T]ATTTAGCATAGAGCAGCTTC |
| 92 | | 6 | GCAAAGTTCATATAT |
| MI13 | 9 | 12041744 | TATACAACGCTGCTTTCTACCAAGTTAAACGAGCT[A/G]ATGTTTCTTTTTTTTTATCAT |
| 93 | | 4 | CAAAATCGACTGCA |
| MI13 | 9 | 12087933 | AAGTCCAAGAAGTTGGAACCTGAACCGGAAGCCAG[A/C]GATGTTAGAGAGGTTTTT |
| 94 | | 8 | AGTTTTTACGTTTCTAA |
| MI13 | 9 | 12184457 | GCAGAAGGCCGGCCGGCTACTCGAAAACAAGAACA[A/C]AGGAATCACAGCTGGGG |
| 95 | | 3 | CGCTATACCTACACACCT |
| MI13 | 9 | 12218789 | AAAATACTACACTTTTAAACAGGGCCTATATGTGG[C/T]GGTGGAACTACGTGGAACT |
| 96 | | 0 | AACCCTCTTATCATGG |
| MI13 | 9 | 12452199 | AACATTAATTTTACAAATACAATGTCATTCATTTC[C/T]AGAACATCAAAATTTAGTTGC |
| 97 | | 9 | TGAAATGAGCTTAT |
| MI13 | 9 | 12457286 | GACCGATGCTCGTCGACACGGGTACGGGGCGAGTC[A/G]GTCTGAGATACGGTGGCA |
| 98 | | 1 | CTTGCCACGCTTAGGTT |
| MI13 | 9 | 12575650 | TCTAAGTGTCTCCGCAGCCATTTGAGGGACAACCG[A/C]TATAGCAATGCTATCATGCA |
| 99 | | 7 | CGCATATGCATATGG |
| MI14 | 9 | 12747552 | ATAATAGTTTAATTCGTGGGTCGTAATTTTGTTTC[G/T]GACTTGAAACGCTTGCGCAG |
| 00 | | 8 | GCAGACAGGCAGGCC |
| MI14 | 9 | 12795064 | GGACGCTATTTGCAAGCTGAAATCGACGTAGGCGG[C/T]GAAATCGCAGTAGCAAAG |
| 01 | | 9 | ACTGCTGGGCCAGTAGA |
| MI14 | 9 | 12817985 | GGCATCGATAATGACGAAATGATGGCTTGCGCTTC[C/T]AGCCTCCGACTGCATTCAA |
| 02 | | 8 | GGCATCGATAATGGCA |
| MI14 | 9 | 12867316 | GACAGCAAATATGGTTTCAACTTCGAGGTTGCAAA[C/T]AGTGAACATTGTAGTAATA |
| 03 | | 1 | GGATCTACAAGGGTGA |
| MI14 | 9 | 12976485 | CAACTCTAATCGATATACTCCCTCTCCCTCGTCGC[C/T]TCGACCACATATAGAACTCGT |
| 04 | | 3 | CCATTGCTTCGTCG |
| MI14 | 9 | 13383791 | TTGATTTCTCTTGTGTTGGTTATACATATTTCTTT[C/T]GCGAAGGAATGACAAGGCACT |
| 05 | | 9 | AATTAGCGCTAGCG |
| MI14 | 9 | 13394714 | CCGGACCAGATCTTCAGACCAGCAAAGCAAGGAGA[C/G]GATGCCGTGCCAATGCCA |
| 06 | | 0 | TGCTCCAGTTGGGTGGC |
| MI14 | 9 | 13614391 | TCCAGTTTAACCAACTGGTCATGGCAAATCCTGAA[C/T]CCCGTTGTTAGGTGATTACT |
| 07 | | 1 | ACCGGCAAACCTTTT |
| MI14 | 9 | 13804120 | CCTTTTCCTAAAGACGTACAGATACGATCGAGGCA[C/T]TACACAGACACGTACGCTG |
| 08 | | 5 | GTGACACGTAACAATA |
| MI14 | 9 | 13810406 | CTGATCTGCTGAATTCTTTCGTTCCTAACAGTTTT[C/T]CCCCCAACCACAAGAAATTG |
| 09 | | 7 | AAGAATTGAAAAGTG |
| MI14 | 9 | 13826921 | CTCAGCTATAATCTGAAGCGGTCATTTCAAAAAGA[A/C]TGTCGCACCATCACCTAAA |
| 10 | | 5 | CAACGGGCTACTCTTC |
| MI14 | 9 | 13946970 | ATAAGAAAAATTAATGGTCAAAAACTTTTGTATGA[C/T]TTTTTTCTCTCGTCTAATATG |
| 11 | | 2 | ATTAACATTTTTCG |
| MI14 | 9 | 14059889 | TATCATTAGTGGAAGCCCTTCTTTTGTTACTTTTT[C/T]TATACCTTGTACGCTTATTGA |
| 12 | | 9 | CATGTCCTCATTAG |
| MI14 | 9 | 14103836 | CATAAGTTCATTGTATATAAGCAAGTAAATACAGG[A/G]AAATGTTCCCTTTTTGAGAG |
| 13 | | 8 | GAAGATACATAAAAA |
| MI14 | 9 | 14210399 | GGGTGAGATTGTGTATAGAACACTTTTGGGCTTTT[A/G]GCCAATTGTGTTGCTGCAGC |
| 14 | | 1 | CACATAAAAAATCAG |
| MI14 | 9 | 14307224 | GATGATGCACGGAACCCAACTGAACGAAAGACGAG[C/T]TGATATGGCATTGTTTGCC |
| 15 | | 5 | GCGCAACGCATCCGTC |
| MI14 | 9 | 14308023 | TCGAGATGTGCTAGACCGGACGCTCAACTACCGGG[A/C]TGTCGTCGTCGGTTCGCTG |
| 16 | | 5 | TCGGGTTATTTGAACT |
| MI14 | 9 | 14346603 | ATCAATCATCCTCAGTTCAGCTAGCGAAGTGATAC[C/T]ATGAGTGCTACATCTATGTA |
| 17 | | 9 | ACTTCATGCCATGTT |
| MI14 | 9 | 14477457 | CACATGATGTGCTTGGAAGAATTCTTAGACGTGGG[C/T]TCTTGGATGTTGAAGTAGAT |
| 18 | | 3 | TAAAGAAGAAAACTT |
| MI14 | 9 | 14478694 | GTTGTGTGGCACGATTGCGACATTATTGATCTTGA[C/T]GATTCTGTGAATCCATGGATA |
| 19 | | 8 | TAACGTAGTGTGTG |
| MI14 | 9 | 14537735 | GTTGGGCTAAAGTCTGTTGGCGCATAGTGAAGCTG[A/G]GTTTAGAAGAAAAAAAAG |
| 20 | | 0 | GTGACGATGGTGATGGT |
| MI14 | 9 | 14559303 | TATGCACCTGGACAGAGCCGTAGTCCCTTATATTT[G/T]ATAGGATGAAGGCAGTACAT |
| 21 | | 2 | GTTGTATGGTAGTAG |
| MI14 | 9 | 14602413 | GTGACCATATGTTGGGGCTGCAAGTTAAGAATAAT[A/G]TTAAAACAATTTATCAGATG |
| 22 | | 0 | ACTTGCTGAATAAAA |
| MI14 | 9 | 14753826 | CAAAGCATGAATGCTAAAAGTTACTGAAAGTGAAT[C/T]GCTAAACTTTAGCACCCAA |
| 23 | | 7 | CAACAGTTTAGCTACT |
| MI14 | 9 | 14780584 | GTATGTATCTCACCTGACACAGATGTGGCCTCCAG[A/C]CAGGTGTAGGATATGGATGC |
| 24 | | 3 | TGTGGACATAAAAAG |
| MI14 | 9 | 14815508 | CGTCTATGAGGCTATGACAGATTAGTCATGTGGCG[A/G]GAGTACAGAACACGCGTGA |
| 25 | | 7 | CAGGTTAGCAACCACC |
| MI14 | 9 | 15016351 | CTTTGTGGAATCACAGACTCTCTGTTTCAGAAAGC[C/T]TGACGTTTTCACCCAAAGG |
| 26 | | 4 | GTAGCTTAGGCTAGCC |
| MI14 | 9 | 15133947 | GTGCGGCAACTCGAAACTCGTGTGTCTTGGTTACA[A/G]AAATTAACGACGACCCTCG |
| 27 | | 1 | GTGCCAGAAGCATGAG |
| MI14 | 9 | 15151663 | ATAATTTTGGAAGAACGCACTGTTGCAATGCATGC[A/C]TGACGTTTTCCATCCAACTG |
| 28 | | 6 | CTAGGGTCCAGCACG |
| MI14 | 9 | 15272782 | GCTATACATTGAACTCCCTGTTCACTAGCAGCATC[A/G]TTGACAACAAAAGCACCAG |
| 29 | | 8 | CTTTCGCTGCAGGACC |
| MI14 | 9 | 15343143 | CTTGTACTAGCGCTGCTCTGTTCTTAGCAGCTCAA[C/T]GTAAAAAAGCAACTTTGTTA |
| 30 | | 1 | TAGCTCAATGTATAA |
| MI14 | 9 | 15595386 | TCTCCAGCACGTCCACCACCGGTTCACTTCCTCAT[A/G]TGTTCAGGAACGATCCGAT |
| 31 | | 6 | GGACAAGCTCGATTGG |
| MI14 | 9 | 15668272 | GTCCGTAAAGCGGTTCTAGGGTGTCGCATTATGGG[A/G]ATAAGGGAATGAAAAGGCG |
| 32 | | 2 | GCTGGTTGTGGGCGGA |
| MI14 | 9 | 15687369 | GAATTCAGCTAGATGCTTCCTTCGCCTCTGGCCTC[C/T]GCCTTCACTCAAGTTCTTGG |
| 33 | | 5 | ATGTTATCGTTGCCA |
| MI14 | 10 | 1150024 | CCGCTCATGACGTAGTTCCTCTGATCCACGTTGCC[A/G]AAGAAAGAACCTTCCATTT |
| 34 | | | TTTGATGCACAAGCGA |
| MI14 | 10 | 2667299 | AGAGAGAAAGCCCTATGTTTGGTTTTGTTAGTGCT[G/T]TTTCTTTGCTGGTCAAAACA |
| 35 | | | AATATAGTGCTTGAT |
| MI14 | 10 | 9274307 | CCATGAGGTATACGAGGCAGAGCCCTTCGGCCAGT[C/T]GTGTCTCTCGTGGCTAGAG |
| 36 | | | GTCCAAATCACGTTTG |
| MI14 | 10 | 9366532 | GGTGGTGTCCGAGCAGGTCGTTTCATGGTTAGTAC[C/T]TCGGCCAAATTGATAACCAT |
| 37 | | | AGTGTAATTCCACGT |
| MI14 | 10 | 11246795 | AATGTGCCCCATCATGTGGGCAAATGCCATAATGG[A/T]TTGCAGTCTCTTTCTGTACT |
| 38 | | | TTATCACGGTAAATT |
| MI14 | 10 | 12720998 | GTGGTGGCGGCCGACGTACGTGCAAGCAGATTGAT[C/T]AGAAAGAGAGAGGATGGA |
| 39 | | | TTGGTTGGTTGTTGTTA |
| MI14 | 10 | 13172473 | AACTTAATTTCCTGCAGAATAAGAATGTCAAAAGC[A/G]TCAGAATATTCAACTTGGG |
| 40 | | | AAGATCATCACGACAT |
| MI14 | 10 | 13562279 | AATCTCTGCTTGATGCTGGTGGATAGTGGGGGAAG[A/G]TTTCGATGAGCGGCTGCTG |
| 41 | | | GTGGAGCCGGGGAGAG |
| MI14 | 10 | 13791427 | TAATTTCGTTTCAGTGATTCACAGTACCTAGCTCG[C/T]GAAACCACAAATTCAGCTGG |
| 42 | | | ACTAGTCCTGCAGGA |
| MI14 | 10 | 14829973 | CCCCATTATTATTTTTCCATCAATCTCAATATTCC[C/T]TTGGATCTCTCTCTTTCAAAG |
| 43 | | | GCAAGGGTACACTA |
| MI14 | 10 | 15400809 | GCTTGCCTTGTCACTTAGCAGGTCTGTATTGCTTG[C/T]TCGGTGCCTGGCACCTTCTG |
| 44 | | | TAACATTGTTTTTGA |
| MI14 | 10 | 17390767 | GTCCCAACTTCGAGGTTCCAACGATGATGAATTCA[C/T]GGTTTAATGGTGGTGGCAA |
| 45 | | | GCTTTTCTCCCCCCTG |
| MI14 | 10 | 17785477 | TGGAAATCAATTTCCACCGGTGGTTTTTAACTAAC[C/T]GCCAATGAAAAGACCGATTT |
| 46 | | | CTACATACCCTTAGT |
| MI14 | 10 | 21828268 | CGTACTGGACGACCAAAACGCTCTGAGTATTCTAT[A/C]TTCAACAAGAAATACCACG |
| 47 | | | GAAAAACGCGGCCATA |
| MI14 | 10 | 22275001 | ACGTATCTACAACCGCTTGTACGCGGTCCAGTTGA[A/G]TGGAACAAATGATGCTATTC |
| 48 | | | TTCTTGAAGCTGCTA |
| MI14 | 10 | 24386964 | TAGCTATCGACCGGTCACTGACGTGCGAGACCAGC[C/T]GTTGCTTCGCCCCGTACCT |
| 49 | | | CAGTGGCTGTAAGTTA |
| MI14 | 10 | 24558753 | CTCAACAACACCCACACCACAAGAACTTGATCGTT[C/T]TTCCACAAATTCAAAGAGG |
| 50 | | | AGAGGGAAGAAGTGAA |
| MI14 | 10 | 26101648 | CTCCCCCAATTGCTACCTGTGGTTCACTCCTATCT[A/G]TGCTTGCTGCTACATGTTATT |
| 51 | | | TGAATTTCAAGTTG |
| MI14 | 10 | 26360071 | AATGCTAGTACACTTTGGACATCCTAGAGTGTGCT[A/G]GCACGGTGGACTGTAAGTC |
| 52 | | | GTGTGCTACTTCGGTG |
| MI14 | 10 | 27960831 | CAGACTCGCTAAAGGGACACGTATGCCGGACAAAC[A/G]GAGCGGTACGCATACTGG |
| 53 | | | GAGTACGATTATGCACG |
| MI14 | 10 | 29812819 | TAACAACACCAAAACGTGCGAAGACGCTACCATCT[A/G]CAACTCAGCGCATACACC |
| 54 | | | ACAATGAAAGACACATC |
| MI14 | 10 | 31334036 | CGTACTCCTGGTATGGCATATCTTCTCGCTCATTT[G/T]TCATATGGACCCCTGTAGTTA |
| 55 | | | GTCCATCGCTAACA |
| MI14 | 10 | 35046490 | AACTGAAGGTATCACAAAAATAAGTTGTATTTGCT[C/T]ATTGCCAAAAATCAAGTCAT |
| 56 | | | ATCAAAGCAGGTAGG |
| MI14 | 10 | 35156008 | CGTTTCGGTTGCGTCAACTTTCTAATAGCCTCGCC[G/T]GTGTGTTAAAACATTATTTTG |
| 57 | | | TCGCGACACAAGTT |
| MI14 | 10 | 35865983 | GTGGTCGCCGATGAAATATCTCCGGTCAGGCTCCA[A/G]CAAGGTTCGCGGAATCATG |
| 58 | | | ACACGGCCCGTAGTTA |
| MI14 | 10 | 38050344 | TTTGAAAACCATAATGTCAGAGAGGAAAATCACCA[A/G]CTGTGTGGTGGAACACAC |
| 59 | | | CCGCCCTAAATCCTAAG |
| MI14 | 10 | 43624157 | GTGACCGAAGTGCACCCAATTGATCTTCACCGTCA[C/T]GCATCCGTAGTTCGCCGTG |
| 60 | | | GACTGACCGAGCCAAC |
| MI14 | 10 | 45691464 | CATGTCACCTTCGTGGATATAAAAATGGATTAAGG[C/T]GGAAAGATCATTAACCGCCT |
| 61 | | | CTGATACCATATGAA |
| MI14 | 10 | 48353351 | CTTTACACCGAGGACCCTAGTCATCCAAATAACCT[G/T]TAAAGAGTCCTTAGCACTAT |
| 62 | | | TGCTCTGTGTCTCTA |
| MI14 | 10 | 53559947 | TGGGGCAAATCATGCGTAATATAAAGATAAGACAC[A/G]CAATTAACCTTATAAGGTAA |
| 63 | | | TTCTCCTCCTCCTTA |
| MI14 | 10 | 55197135 | CTTCAAGATGACTCCACTTTGTTTCTTTCGATGAA[G/T]TTCGTGGATAGTCTCATGAA |
| 64 | | | GAATGACAACCCCTT |
| MI14 | 10 | 59526327 | TGCGGCCTAACTCAGCTCGTCGCCATGCGGTATGG[A/G]ACCATCCCGATTGTCCGCA |
| 65 | | | AGACTGGAGGTACGTA |
| MI14 | 10 | 59821677 | TAGCCTAGCAAAATCTGTTCAAATGCAAAATCGTC[A/G]AGAACAAGCAATCGACACA |
| 66 | | | AAGAAATGAATAAAAT |
| MI14 | 10 | 63829892 | CAAACAAGGCTGTTACTAAGAAAATGGTGTTTACT[A/G]CATTAAGGGCTAATTTCGA |
| 67 | | | AACTCAAATCTCCTTG |
| MI14 | 10 | 66187229 | AAACATCCCTACAAAGACTACAAACATTGCAGGCA[C/T]TGTCCATGTTGTGCTGCTG |
| 68 | | | GATCGAAAAATCCAAC |
| MI14 | 10 | 66645618 | TAGGATTGGGTTAAAATTGGAACCGAGTTTAGTGC[C/T]ATGGATGAATGAGTGTTTCA |
| 69 | | | CATTGTTATCGCGTC |
| MI14 | 10 | 66652391 | GCTGCCATTCCCGTAACCAAGATTTGGGTATATAA[A/T]GGAGGCGTTTAAACGACCA |
| 70 | | | GTCTTAACCGCGACCG |
| MI14 | 10 | 67781960 | TTGGTCTATTTGTGCAAATAACTACATGTTTATAA[C/T]GTCCCGCACTATTGTGTATTG |
| 71 | | | TGGTCGACAATGCC |
| MI14 | 10 | 71744694 | TGGGATATCGGAGGAGAAATTTAGGGGGAACCGCT[A/G]AACACAGTCTAAGGCTGA |
| 72 | | | GCCAGCTGCAGCTTGCT |
| MI14 | 10 | 72720178 | CCACCTGATTCATCAAAAAATTTGACGATGGAACA[A/G]CATTAAGTTCAAATCACCT |
| 73 | | | TCAAGGCCACCGAAAG |
| MI14 | 10 | 73217544 | CGCCACATCACAATTCACAAACAAAAAACCATGCC[C/T]CCCCTAGATCACAGTATCA |
| 74 | | | GCAGAAAATCCGTCCA |
| MI14 | 10 | 73219081 | AGCGTGCGAAACATGCCACGCTCTAAGGAGGCCCT[A/G]TTCATCACCAGAGAGTCT |
| 75 | | | TCTTGGTTGAATCCCTG |
| MI14 | 10 | 75265737 | CTGCCCTCCTGCTGAAATCAACAGCTTATCTCCTA[C/G]GTGTGGATAGTTATTTCATG |
| 76 | | | AGTCTCTCGAATCAG |
| MI14 | 10 | 77204470 | GCTGAGCGTTGACTGTATATAGAATGCTATTTTAT[C/T]TTTACCTTTTCTAAGTACGTA |
| 77 | | | ATTGACTGACAGAG |
| MI14 | 10 | 79386470 | CTCTTAAATGAGTCGTCTGTATGATGCCATGTCAC[A/G]GAAATTTGCTTACGTGGACT |
| 78 | | | ATAATAAAAAGAAGG |
| MI14 | 10 | 82054207 | GCTGCAGCAGCTTGTGGCTGGAATCTTGACACAAC[G/T]GTAAGGAGCTACCTTTTTT |
| 79 | | | TTTCAGTTTGATACGC |
| MI14 | 10 | 82056607 | GTGCGCTGCTCGAGCGGTTCATAGCGTCGAGACCA[C/T]GTCAGCAATAACGCACAAT |
| 80 | | | TACATAGAATCTATTC |
| MI14 | 10 | 82632955 | TTCATCAATCTCGGCCCTTTGATCTGAAACTAGCG[G/T]CCATGATTAGATCTAGTTTAC |
| 81 | | | AGCAAACCGTTAAG |
| MI14 | 10 | 86365408 | TGCACTTGCCCAACAATGTGGTAGTCAAACTCGTG[C/T]GCGACATTGAGGTGGCTTC |
| 82 | | | CTAGATAGGATAATCT |
| MI14 | 10 | 87006666 | CCGAGCTGGAGAAACAGTGAGTCGGGGAGACGAGC[A/G]GCAGTGCAACCCCTGAG |
| 83 | | | GCGGTGAAGCAGCGAGAC |
| MI14 | 10 | 87181981 | AGCATCTAATGAAGATATGTTTGAACATTTTTATG[G/T]GGTGTCGTGCATCATTAGATA |
| 84 | | | CTCTGGATAAAAAT |
| MI14 | 10 | 87282119 | CTAAAATTTCTATATAGTTGAACAGGAAATGAAAC[A/G]CTATACACGATAGAAATCAA |
| 85 | | | TCACCACCCCCAACC |
| MI14 | 10 | 88282506 | GCGTGGATTAACATAATTATGACTATATATCCAAA[C/G]ATGACTATTTTCAGCATAGTC |
| 86 | | | ACGGACAAATTGTA |
| MI14 | 10 | 89540064 | GATGTTTGGTTGTTTGTTGGGTAACATATGTCCTC[A/G]TTATAGTGAGTCTAAACTTAG |
| 87 | | | ATGATTCACATGCT |
| MI14 | 10 | 91108119 | CTATATTTATGAATTGAAAGTTAGAAAAATCAACT[C/T]CTATGAGACATGTAGCAGTTT |
| 88 | | | CATAGATACAAGGC |
| MI14 | 10 | 92751666 | TTTAGTTTAATCGATGTAGATTAATCTTTCTAGCC[A/G]AAGTAGATTTGCTTATAATTA |
| 89 | | | TAATCTGACTACAT |
| MI14 | 10 | 93827070 | AGCTAGCACAGCTAGAGGTAGTTGACTCTGCTGTA[C/T]GTACGTACGTATGCTATGAT |
| 90 | | | GGAGGGCGACGACTT |
| MI14 | 10 | 95055628 | AGAGGCTGTTGACGAGAGGCCCCAAGAACATCCAC[C/T]GCTGGCCCAGGTTCCACG |
| 91 | | | CGAAGAGGTACAAGCCC |
| MI14 | 10 | 95056435 | ACGAGCCAGAAGAGGACGTCCACGTTGAGGAAGAA[G/T]AGGACGTTGGCCAGGAC |
| 92 | | | CAGAGGGTGCGCTGCGCA |
| MI14 | 10 | 96024147 | GAGGAGGGGCATGAGAGAGCTGAGGTAAGGCCCAC[C/T]GCCGCTTTCCTAGCACCA |
| 93 | | | GCGCTGGCCTGGCCTCC |
| MI14 | 10 | 97469203 | GACGAGCTACTATGGCGGATCAGGTGCCCGGTCGC[C/T]AGAGTTGAGCACGGTGTTG |
| 94 | | | TCATGGCCGGGAAGTC |
| MI14 | 10 | 10321685 | GAAAGAAGGATCTATTCACGAGGAGATGAGTTAAA[C/T]GTAAGGTACCGTTTGGTTC |
| 95 | | 6 | CAAAACATAGTTGGAC |
| MI14 | 10 | 10530112 | TAATTTTCTAGCACATGCAAATAAAAACAAAGGAT[A/G]CACATGTAAGGAATGGGCG |
| 96 | | 9 | CGTGGGGGTGGAGGCG |
| MI14 | 10 | 10811813 | AGCACGATAGCTGACAGCCACACATATATCTACGG[C/T]ACGATGGCCGACAATCACAT |
| 97 | | 3 | CAATCGGCGTCCATA |
| MI14 | 10 | 11010437 | TACAACTGGTGATCACATAGTTTTTGCAAAGCAAG[C/T]CTCAAGTGTTTCTTTTTTTC |
| 98 | | 0 | CTTTCTCATTCTTGG |
| MI14 | 10 | 11073564 | GATGGTGTGAGGGGACGGATGCGGTTGTTGGATCA[A/C]CATACGGTGCTGCCGGAG |
| 99 | | 9 | CTAAGGAAGAAGATGCT |
| MI15 | 10 | 11178715 | GTGGGTGGTTGGACTGGTTTGTCGAAGGGCTAGGC[A/G]AACCATGAAGGTTGTTTG |
| 00 | | 3 | ATGTCGCCGATTGATTC |
| MI15 | 10 | 11318842 | ATTATCTCACATCGAGAGCCCAAACTTGTATAAAT[A/C]TGTGTGTCTCGCTTTCGGTC |
| 01 | | 1 | ACGTGACTTACCCTA |
| MI15 | 10 | 11373234 | GCAGGACGTGTGGGTGTGGCTCGACCTCAGTGGAA[C/T]AACAAAGTTTTCCTCTGC |
| 02 | | 1 | TTACCTAAGCTAAGACC |
| MI15 | 10 | 11384698 | TGATCCTTGCATGAATCCTTTGCACGCATGGTGTG[G/T]CGACCGCTCCACCTCCTTTC |
| 03 | | 7 | CTCACTCGTTGTTTC |
| MI15 | 10 | 11595829 | TATAGCATTATTATTAGGATAGGATATATGTCATG[C/T]AATAAGTTAATGTACATGAAC |
| 04 | | 6 | TACCGTATTGCAGC |
| MI15 | 10 | 11645215 | TTCTTGTCGATACTTGGTCGTTTTTGCAGAAAACA[A/G]AAAACATTCATGTGTTTTTG |
| 05 | | 3 | TAATCCAACAGTTTA |
| MI15 | 10 | 11654638 | AGTGTTTACTTTGGCAAATTAACTTAATTTTGTTT[C/T]TTGCACTACTTTACAACTTTG |
| 06 | | 1 | TTTCTATTAACAAC |
| MI15 | 10 | 11885883 | CTGTATATTGCGCTTTGACTTTGATTAGATTTATA[G/T]GAAAAAAATTACATAAATATC |
| 07 | | 1 | TGCAGCTGTCATTC |
| MI15 | 10 | 11904668 | ATTTTCTTGAACAGAGATGTATGTAGATCATTTTT[A/C]TTGAACACGGAGGAGAGCTG |
| 08 | | 2 | CGTATTTTTTATATT |
| MI15 | 10 | 12085097 | TCGACGCGAAGTGCCTGCATGACTCGATCCAAAAC[A/C]GCAACTTTTTTTTTTTGTA |
| 09 | | 4 | GTGTTCCTGTCACCTC |
| MI15 | 10 | 12229468 | GAAGTTCTCTGTTCTCTCCCTCGTCCGGTCGTCCT[C/T]GTCCCTCACTCCCTCTCCCT |
| 10 | | 2 | AGTTGCCTACCCCCG |
| MI15 | 10 | 12312508 | GAGGATGTCGAGGAAGGAAAGACCTAGTCCATATC[A/G]ACGCGTCGACGTGGATGA |
| 11 | | 9 | CCGGAGGGAGAGAAGAG |
| MI15 | 10 | 12364545 | AGCCCATGACTTGTATCGAGCTAAATCCTTACTTC[A/G]GGAAAAAATAACTCATGAG |
| 12 | | 2 | ATCTATTGTTTCCGAA |
| MI15 | 10 | 12383726 | GAGCTGGGGATCGGATGTGGGGGTCGTGTACATAG[A/G]CGTCAAGTCGTCAACACTC |
| 13 | | 3 | TCCAGCACTTTGATGC |
| MI15 | 10 | 12510733 | ATAAACCTAACATGGCTAGTGACTAAATTTCATTA[A/G]TTGAACTGAGATTGAAAAGC |
| 14 | | 6 | TCTGTCGCCTAAGGT |
| MI15 | 10 | 13009775 | TTGTTTAGTGTTTCCATGTACACTGTATTTTTGAC[A/G]TGTTCACGTTGGGTGTGATCC |
| 15 | | 8 | GATTTCTTGTTTTT |
| MI15 | 10 | 13241786 | AGGAAAATCATTGACATTTTAAACTAACAGCACAA[G/T]TAATCTCCTATGTTTCGCAT |
| 16 | | 4 | ATGCCTACAAAATAG |
| MI15 | 10 | 13333379 | AGAATAGATATAAACCACAGCGTATTGAACACCGA[A/G]TGTTGCCTGCACACCCAAT |
| 17 | | 6 | AGTACTCACTTGCAAA |
| MI15 | 10 | 13381301 | GCTCGGCTCGTTCAGTGGCGCCGATGCAAAATCAC[C/T]AGGGCCGTAGGGTACTAGC |
| 18 | | 2 | AGATTCCGCGACACCC |
| MI15 | 10 | 13408223 | GCGGCGTCATACCCGTAGCTCCAAAACTTGGCTAC[A/C]TGACCTGAAGCATTTCTATC |
| 19 | | 8 | ACGATTGTTAAACGT |
| MI15 | 10 | 13609986 | AGTGATGGTTCAGCAACTCCGACGGCACGTGTGCC[A/C]GTGTATGTAAACGCGTGCG |
| 20 | | 6 | TGATAGTACATTTTAG |
| MI15 | 10 | 13690740 | TGTGTGATCGATCCATAGGCTTTGCTGTTGCTCTG[A/G]TCCACTGATCTCTCTTCCTGT |
| 21 | | 8 | GGTGTCGCCTTTTG |
| MI15 | 10 | 13696502 | AAGGCCTTAGCTAATTTATTTTTAGCTAGCTAACA[A/G]TCAACTATAGCTTACGTGAG |
| 22 | | 4 | CTCTATTTAGGTGCA |
| MI15 | 10 | 13785408 | GGTCGTGAAGAACTCGGGCCAGCCACAAGGTACTG[G/T]GATATCTATTTGCTTTTCC |
| 23 | | 3 | CGCTTATTATTTTCTG |
| MI15 | 10 | 13837309 | AGGTGAGGTGTGCGACTATCTCTCTCCTCCTCCAC[C/T]GGCCGAGGTACTTCTGGCT |
| 24 | | 2 | GACCGACTAAGGGTGT |
| MI15 | 10 | 13848139 | ACTCCCAACAGTAATGTCGTCTCTGGGATCCTGAC[C/T]GAGACTTTTCACTTTGAGCT |
| 25 | | 2 | GGCACCGAATCAGTA |
| MI15 | 10 | 13905644 | GGGAGACCCATACTCGCCTATCTGCGCCTCCTCCT[G/T]CTCTGCCTCTTGCCCTTATT |
| 26 | | 7 | GCTCTTTTGCAGTAG |
| MI15 | 10 | 14080114 | GGACATATCCTTGTTGTCTGGATTGATGTCAATTA[C/T]AACAGATTCATCGACATACG |
| 27 | | 3 | GGGTCACACGTGCAC |
| MI15 | 10 | 14204600 | ACGGATAAAAGTAATAGAAAAAAGCATAGAATTTT[A/G]TTACACAGATTCGATTATAG |
| 28 | | 2 | GTTGCTGGAACATGA |
| MI15 | 10 | 14313607 | CCGTTCACTTCCTTTTCTCTCAAGTCGAGGGACAT[G/T]GCACCATCCAGCCTCCTAAA |
| 29 | | 1 | CTGTATGCCATTTCT |
| MI15 | 10 | 14353853 | TTTCTAGTTTAATTTAGTATGGATCAAAGTGTGTA[G/T]CAGGTCTCTATTGATTGTACT |
| 30 | | 9 | ACCAAATAAATTAA |
| MI15 | 10 | 14555293 | GGTCTACTAGAGGATCGGTTCGTGTTCATGGCATG[G/T]GTATTAATGTAAACGTTATC |
| 31 | | 3 | GGTAACCGCCAGAAT |
| MI15 | 10 | 14572929 | TAAGTGATATATCATGAGGCGAAACAAAGTAATCT[A/G]TGTTTCCAAAAGATTTCTCT |
| 32 | | 9 | GACAAAAAATTCTAG |
| MI15 | 10 | 14602036 | GCATTCTGTCGACCCAGGCACACGGGTCCAAAATC[C/T]GAGAGGCTAGTGGGTCCA |
| 33 | | 6 | GCTAGCTACAGCTGCTG |
| MI15 | 10 | 14697472 | CTGGTTGTGATATTTTTCATGGCTTGTGAATTGCT[C/T]GGGAAGAGTTATGTGAAACT |
| 34 | | 4 | TATTAGAGTACGGAT |
| MI15 | 10 | 14805072 | AAGTAGTGCAACAGATAACGCCGTTGCATTTCCTC[C/T]AGATTCCTTACCTGTGGCCG |
| 35 | | 3 | TCAGCAAGTGTCCGT |
| MI15 | 10 | 14924545 | CCATACAAAATAAGCATATATATAGTTATAGTCGG[A/G]GAGTTTATAGATTCCACAATG |
| 36 | | 3 | CCATGCCTTGGGAC |

A selection process of the 1,536 SNP loci in the present disclosure is as follows:

(I) Evaluation and screening of SNP markers in Maize6H-60K array.

(1) Genome-wide resequencing is conducted on 400 broadly representative inbred lines inside and outside of China, with a sequencing depth of 10×. The sequences are aligned to a maize reference gene sequence using bwa software, and a total of 27,648,399 SNP loci are mined.

(2) Biological information analysis methods are used to analyze whether the loci are dimorphic and single-copy and have polymorphism, and the number of SNP loci is 183,538; high-quality loci (85,231 SNPs) in the known arrays of maize are integrated together; a set of candidate loci is formed.

(3) The above loci are submitted to Affymetrix and Illumina, two mainstream technology platforms, for locus scoring and probe design; evaluation scores are 0.6 and above, and inclusion is defined as the evaluation results of flanking probes designed being either recommended or neutral.

(4) The loci that meet the design requirements are further evaluated based on the sequencing data and further analyzed to obtain a set of candidate loci (88,619 SNPs) based on parameters such as polymorphism, heterozygosity, and conservation of flanking 60 bp sequences.

(5) The above-mentioned loci are formed into a locus set according to the principle of substantially uniform physical and genetic distribution; locus evaluation and probe design are performed again by submitting to Affymetrix and Illumina again to form a high-density 60K array (Maize6H-60K).

(II) Evaluation and screening of 1,536 high-quality and efficient SNP markers.

(1) Based on the 60K array, using 200 inbred lines and 200 hybrids, loci are verified according to the test results such as data acquisition rate, genotyping effect, and characteristics of triplet. The data acquisition rate is more than 99%; evaluation of triplet samples conforms to the genetic law; the loci can be automatically typed, and evaluation type is poly high resolution (PHR) in each test; the number of obtained markers is 15,031.

(2) Based on the locus polymorphism, MAF value is greater than or equal to 0.2, and PIC value is greater than or equal to 0.2, and the number of obtained markers is 14,630.

(3) According to the fact that the loci are distributed within a gene and adjacent markers are not closely linked, 9,343 markers are obtained.

(4) Still further, in accordance with a uniform distribution, 2-4 markers are screened each 2,000 Kbp, and finally, 1,536 SNP markers are determined (see FIG. 2).

These 1,536 SNP markers can be used in Affymetrix or Illumina's related array technology platforms for production applications (FIG. 1 shows the genotyping effect of a randomly selected locus on the Affymetrix array technology platform).

A probe for detecting the SNP marker described in the present disclosure belongs to the protection scope of the present disclosure.

The present disclosure further provides use of the above probe in the preparation of a genome-wide maize SNP array.

The specific implementation of the SNP array obtained by the present disclosure is performed by the following steps:

(1) Probe and array preparation. Probes for 1,536 SNP loci are synthesized and arrays are produced at Affymetrix.

(2) Sample DNA extraction. Desired samples are collected and sample genomic DNAs are extracted according to the designed maize molecular identification, molecular breeding or other biological research experiments; specific concentrations of sample genomic DNA solutions are prepared according to the requirements of customized SNP arrays and stored appropriately.

(3) Array detection and genotyping of SNP loci. According to the requirements of customized arrays, processed sample genomic DNAs are hybridized with probes on the SNP arrays on the corresponding genotyping instrument to obtain hybridization signal data, i.e., raw array detection data.

(4) Genotyping analysis. Using software, quality control is conducted on the raw array detection data, the data with high quality of the hybridization signal are selected for analysis and converted into genotyping data of SNP loci; combining the data with the corresponding maize molecular identification, molecular breeding, or other biological research experiments, the corresponding data analysis methods are selected to obtain the corresponding results.

Further, the present disclosure provides a genome-wide maize SNP array, which contains the probe for detecting the above SNP marker according to the present disclosure.

The present disclosure provides use of the SNP marker, or the probe, or the SNP array in the construction of a maize DNA fingerprint database.

The present disclosure provides use of the SNP marker, or the probe, or the SNP array in the identification of genuineness of maize cultivars or the identification of maize cultivars.

The specific determination standards for the testing of genuineness of maize cultivars using the present disclosure are as follows:

The genotyping data of test and control samples at 1,536 SNP loci are acquired, and a comparison is made of the data between the samples to obtain the percentage of discrepant loci. The calculation method of the percentage of discrepant loci is D=(the number of discrepant loci n/the number of comparable loci N)×100% (the number of comparable loci must be greater than or equal to 1,460, that is, the percentage of missing data is <5%, and the maximum value of N is 1,536).

(1) The percentage of discrepant loci between test and control cultivars is ≥5%, and it is concluded that a significant difference is detected;

(2) The percentage of discrepant loci between test and control cultivars is <5%, and it is concluded that no significant difference is detected.

The present disclosure provides use of the SNP marker, or the probe, or the SNP array in maize cluster analysis or maize phylogenetic analysis.

The present disclosure provides use of the SNP marker, or the probe, or the SNP array in the analysis of maize germplasm resources.

The present disclosure provides use of the SNP marker, or the probe, or the SNP array in the construction of a maize linkage map and gene mapping.

The present disclosure provides use of the SNP marker, or the probe, or the SNP array in the background selection of molecular breeding materials of maize.

In the present disclosure, SNP loci are mined based on high-quality sequencing data of representative maize samples inside and outside of China, and biological information analysis methods are used to assess whether the loci have polymorphism and whether the loci are single copies, so as to form a set of excellent genome-wide maize SNP loci. Candidate loci are strictly tested and evaluated on the two mainstream array technology platforms; combined with the principles of polymorphism and uniform distribution, a high-quality and efficient combination of 1,536 SNP markers is formed. Based on the combination of 1,536 SNP markers, the present disclosure provides a genome-wide SNP array suitable for molecular detection and molecular breeding of maize, containing the combination of 1,536 SNP markers; this marker combination has undergone a plurality of rounds of verification and has advantages of high quality, high discriminability, high compatibility and uniform distribution; the array can be used in DNA fingerprint construction, cultivar identification and confirmation of maize cultivars, research on germplasm resources at a genomic level, molecular marker-assisted selective breeding, and other basic research, with good application prospects and objective market value.

### BRIEF DESCRIPTION OF THE DRAWINGS

FIG. 1 illustrates a genotyping effect of randomly selecting a locus among 1,536 SNP markers disclosed in the present disclosure. In the figure, a left genotypes cluster is AA, a middle genotype cluster is AB, and a right genotype cluster is BB.

FIG. 2 illustrates the distribution of 1,536 SNP markers disclosed in the present disclosure on the 10 pairs of maize chromosomes, which are well distributed uniformly on the whole genome.

FIG. 3 illustrates a cluster analysis of 200 maize germplasm resources based on 1,536 SNP markers.

### DETAILED DESCRIPTION

The following examples facilitate a better understanding of the present disclosure. It should be understood that the following examples are for illustrative purposes only and are not intended to limit the scope of the present disclosure. Unless otherwise specified, the experimental methods in the following examples are conventional. Unless otherwise specified, experimental reagents and materials used in the following examples are all purchased from conventional biochemical reagent stores.

### Example 1 Obtaining of Genome-Wide Maize SNP Loci and Synthesis of Genome-Wide Array

(I) Evaluation and Screening of SNP Markers in Maize6H-60K Array

(1) Genome-wide resequencing was conducted on 400 broadly representative inbred lines inside and outside of China, with a sequencing depth of 10×. The sequences were aligned to a maize reference gene sequence using bwa software, and a total of 27,648,399 SNP loci were mined.

(2) Biological information analysis methods were used to analyze whether the loci were dimorphic and single-copy and had polymorphism, and the number of SNP loci was 183,538; high-quality loci (85,231 SNPs) in the known arrays of maize were integrated together; a set of candidate loci was formed.

(3) The above loci were submitted to Affymetrix and Illumina, two mainstream technology platforms, for locus scoring and probe design; evaluation scores were 0.6 and above, and inclusion was defined as the evaluation results of flanking probes designed being either recommended or neutral.

(4) The loci that met the design requirements were further evaluated based on the sequencing data and further analyzed to obtain a set of candidate loci (88,619 SNPs) based on parameters such as polymorphism, heterozygosity, and conservation of flanking 60 bp sequences.

(5) The above-mentioned loci were formed into a locus set according to the principle of substantially uniform physical and genetic distribution; locus evaluation and probe design were performed again by submitting to Affymetrix and Illumina again to form a high-density 60K array (Maize6H-60K), which was commercially available.

(II) Evaluation and Screening of 1,536 High-Quality and Efficient SNP Markers

(1) Based on the 60K array, using 200 inbred lines and 200 hybrids, loci were verified according to the test results such as data acquisition rate, genotyping effect, and characteristics of triplet. The data acquisition rate was more than 99%; evaluation of triplet samples conformed to the genetic law; the loci could be automatically typed, and evaluation type was poly high resolution (PHR) in each test; the number of obtained markers was 15,031.

(2) Based on the locus polymorphism, MAF value was ≥0.2, and PIC value was ≥0.2, and the number of obtained markers was 14,630.

(3) According to the fact that the loci were distributed within a gene and adjacent markers were not closely linked, 9,343 markers were obtained.

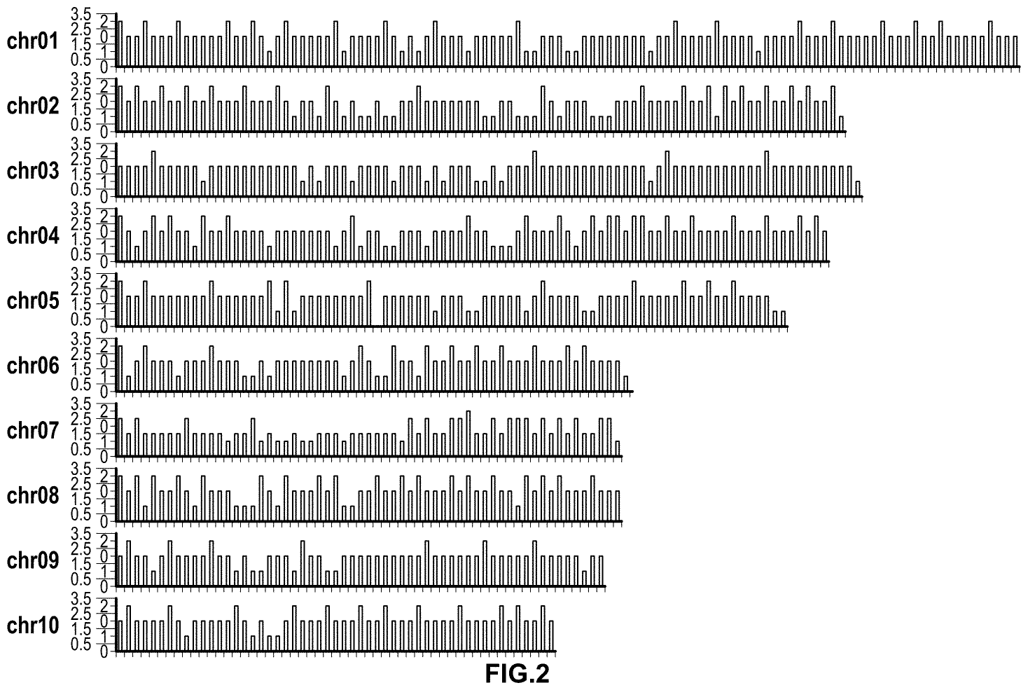

(4) Still further, in accordance with a uniform distribution, 2-4 markers were screened each 2,000 Kbp, and finally, 1,536 SNP markers were determined, as shown in Table 1. FIG. 2 shows the distribution of the 1,536 loci on the maize chromosome.

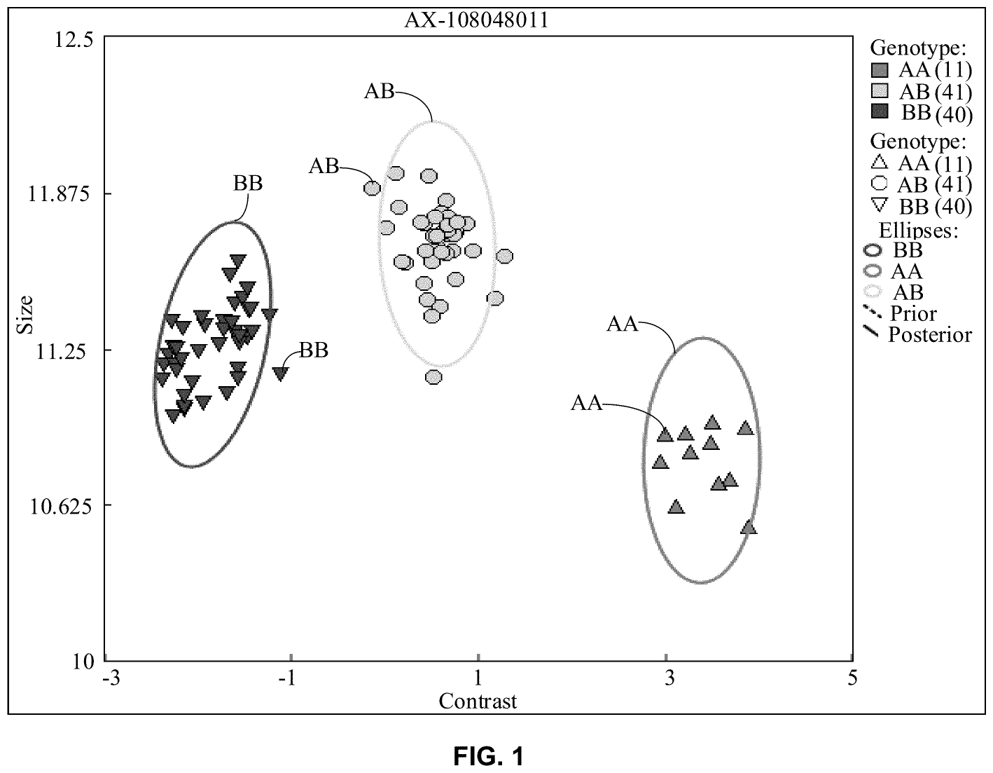

These 1,536 SNP markers can be synthesized and applied using Affymetrix or Illumina's related array technology platforms. FIG. 1 illustrates an experimental effect of randomly selecting a locus on the Affymetrix array technology platform.

### Example 2 Construction of a Standard Maize DNA Fingerprint Database Using the Genome-Wide SNP Array of the Present Disclosure

1. DNA Extraction and Quality Identification

Samples of known maize hybrids were selected, genomic DNAs were extracted separately using conventional cetyltrimethyl ammonium bromide (CTAB) method, and RNAs were removed; the quality of the extracted DNAs was separately detected by agarose gel electrophoresis and ultraviolet (UV) spectrophotometer. That is, agarose gel electrophoresis showed that DNA band was single and there was no obvious diffusion; the UV spectrophotometer detected A260/280 between 1.8 and 2.0 and A260/230 between 1.8 and 2.0; Qubit4 was used to quantify genomic DNAs, and the genomic DNAs of all maize samples were diluted to 20 ng/μl.

2. Genotyping Experiment Using the Genome-Wide SNP Array

The genome-wide SNP array prepared in Example 1 was used to detect the genomic DNA of each maize sample obtained in step 1. Specifically, operations were in accordance with the Affymetrix array detection standard process; array hybridization, array scanning, raw data acquisition, etc. were performed on the GeneTitan system to obtain the raw data for each sample.

3. Acquisition of SNP-DNA Fingerprint Data of Maize Samples

The raw data were imported into Axiom Analysis Suite software, and the fingerprint data of 1,536 SNP markers for each maize sample were analyzed; finally the DNA fingerprint data of the SNP markers of the maize samples were imported into a database management system to establish a known maize hybrid SNP-DNA fingerprint database.

Example 3 Identification of Genuineness of Maize Cultivars Using the Genome-Wide SNP Array of the Present Disclosure

1. To Determine the Criteria for Determining the Results of the Genuineness of Maize Cultivars Using the SNP Array Prepared in Example 1

Agricultural industry standard (NY/T1432-2014), the Protocol for the Identification of Maize Varieties—SSR Marker Method, is a DNA fingerprint identification method for maize cultivars based on SSR markers; this standard deems cultivars with 40 SSR loci with ≥2 discrepant loci as different cultivars, which are calculated as 5% discrepancy according to the locus ratio.

An analysis was made of the pairwise comparison of the percentage of discrepant loci of 400 maize cultivars, and results showed that the percentage of discrepant loci was between 0.2% and 75%, among which maize cultivars with a percentage of discrepant loci of less than 5%, 5%-10%, and more than 10% accounted for 0.03%, 0.025%, and 99.94%, respectively. According to the above results, the 5% percentage of discrepant loci can be used as a determination threshold for the identification of genuineness of maize cultivars.

The specific determination criteria for the identification of genuineness of maize cultivars using the present disclosure are as follows: genotyping data of test and control samples at 1,536 SNP loci are acquired, and a comparison is made of the data between the samples to obtain the percentage of discrepant loci. The calculation method of the percentage of discrepant loci is D=(the number of discrepant loci n/the number of comparable loci N)×100% (the number of comparable loci must be greater than or equal to 1,460, that is, the percentage of missing data is <5%, and the maximum value of N is 1,536).

(1) The percentage of discrepant loci between test and control cultivars is >5%, and it is concluded that a significant difference is detected;

(2) The percentage of discrepant loci between test and control cultivars is <5%, and it is concluded that no significant difference is detected.

2. To Identify Whether a Maize Sample to be Tested is a Known Control Maize Cultivar

The following will describe in detail how to use the SNP array prepared in Example 1 to identify whether a maize sample to be tested (code A) is a known control maize cultivar Zea mays ‘Zhengdan 958’ (code B).

DNA extraction and quality identification: Genomic DNAs were extracted from a maize sample to be tested (code A) and a control maize cultivar Z. mays ‘Zhengdan 958’ (code B) for DNA quality identification, array detection and acquisition of genotyping fingerprint data of 1,536 marker loci. The specific methods were the same as those in Example 2.

An alignment was made of the fingerprint data for 1,536 SNP genotypes between sample A and sample B. According to the results, the number of comparable loci between sample A (the maize sample to be tested) and sample B as the control maize sample (the known maize cultivar Z. mays ‘Zhengdan 958’) was 1,636, the number of difference loci was 940, and the percentage of discrepant loci was 61.2%. According to the 5% determination criteria, the maize sample A to be tested was not identified as Z. mays ‘Zhengdan 958’.

### Example 4 Identification of Specificity of Maize Samples Using the Genome-Wide SNP Array of the Present Disclosure

The following will describe in detail how to use the SNP array prepared in Example 1 to identify whether a maize sample to be tested (code C) is cultivar-specific.

DNA extraction and quality identification: Genomic DNA was extracted from a maize sample to be tested (code C) for DNA quality identification, array detection and acquisition of genotyping fingerprint data of 1,536 markers. The specific methods were the same as those in Example 2.

The fingerprint data for 1,536 SNP genotypes of sample C was imported into the known maize hybrid SNP-DNA fingerprint database constructed in Example 2, and the fingerprint of the sample C was aligned with the fingerprints of known cultivars one by one. If the percentage of discrepant loci between the sample C and known maize cultivars reaches 5% and above, the maize sample C will be specific; if the percentage of discrepant loci between the sample C and known maize cultivars is found to be less than 5%, the maize sample C will not be specific.

### Example 5 Analysis and Evaluation of Maize Germplasm Resources Using the Genome-Wide SNP Array of the Present Disclosure

In the evaluation and screening process of 1,536 SNP markers provided by the present disclosure, various types of representative germplasm resource materials are selected. Therefore, a good discrimination effect can be obtained when evaluating and analyzing maize germplasm resources samples, such as the use in heterotic grouping. The following is the heterotic grouping of 200 germplasm resources samples using the SNP array prepared in Example 1.

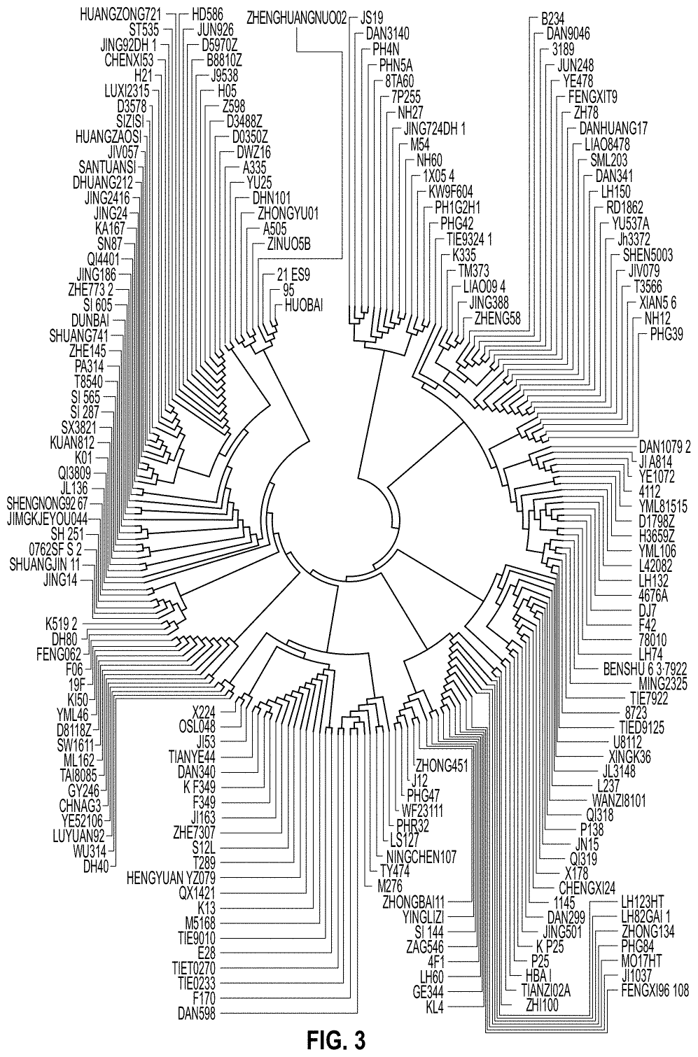

For 200 maize samples, specific methods for DNA extraction and quality identification, array detection and acquisition of genotyping fingerprint data of 1,536 SNP markers were the same as those in Example 2. Based on the acquired genotyping data of 1,536 markers in 200 samples, using Power-Marker ver.3.25 software, Rogers (1972) genetic distance calculation method was used to analyze and establish an NJ cluster-joining tree. Thus, 200 samples were grouped into different groups, and a total of nine groups were formed (see FIG. 3).

The present disclosure has been described in detail above with reference to general descriptions and specific examples, but it will be apparent to those skilled in the art that some modifications or improvements can be made based on the present disclosure. Therefore, all these modifications or improvements made without departing from the spirit of the present disclosure fall within the scope of the present disclosure.

What is claimed is:

1. A genome-wide maize single nucleotide polymorphism (SNP) marker, comprising 1,536 SNP markers shown in Table 1, wherein the SNP markers refer to sequences corresponding to the “sequence” column in Table 1, wherein a nucleotide in square brackets ([ ]) of each sequence represents an SNP locus.

2. A probe for detecting the SNP marker according to claim 1.

3. Use of the probe according to claim 2 in the preparation of a genome-wide maize SNP array.

4. A genome-wide maize SNP array, comprising the probe according to claim 2.

5. Use of the SNP marker according to claim 1 or the probe according to claim 2 or the SNP array according to claim 4 in the construction of a maize DNA fingerprint database.

6. Use of the SNP marker according to claim 1 or the probe according to claim 2 or the SNP array according to claim 4 in the identification of genuineness of maize cultivars or the identification of maize cultivars.

7. Use of the SNP marker according to claim 1 or the probe according to claim 2 or the SNP array according to claim 4 in maize cluster analysis or maize phylogenetic analysis.

8. Use of the SNP marker according to claim 1 or the probe according to claim 2 or the SNP array according to claim 4 in the analysis of maize germplasm resources.

9. Use of the SNP marker according to claim 1 or the probe according to claim 2 or the SNP array according to claim 4 in the construction of a maize linkage map and gene mapping.

10. Use of the SNP marker according to claim 1 or the probe according to claim 2 or the SNP array according to claim 4 in the background selection of molecular breeding materials of maize.
